(19) United States
(12) Patent Application Publication
(10) Pub. No.: US 2016/0222423 A1
(43) Pub. Date: Aug. 4, 2016

(54) ENZYME-CATALYZED ENANTIOSELECTIVE AZIRIDINATION OF OLEFINS

(71) Applicant: California Institute of Technology, Pasadena, CA (US)

(72) Inventors: Christopher C. Farwell, Pasadena, CA (US); Ruijie Zhang, Glendale, CA (US)

(73) Assignee: California Institute of Technology, Pasadena, CA (US)

(21) Appl. No.: 15/006,720

(22) Filed: Jan. 26, 2016

Related U.S. Application Data
(60) Provisional application No. 62/108,300, filed on Jan. 27, 2015.
(60) Provisional application No. 62/120,126, filed on Feb. 24, 2015.

(51) Int. Cl. C12P 17/10; C12N 9/02

(52) U.S. Cl. CPC C12P 17/10; C12N 9/0042; C12Y 106/02004

(57) ABSTRACT

The present invention provides methods for catalyzing the conversion of an olefin to a compound containing one or more aziridine functional groups using heme enzymes. In certain aspects, the present invention provides a reaction mixture for producing an aziridination product, the reaction mixture comprising of an olefinic substrate, a nitrene precursor, and a heme enzyme. In other certain aspects, the present invention provides a method for producing an aziridination product comprising providing an olefinic substrate, a nitrene precursor, and a heme enzyme; and admixing the components in a reaction for a time sufficient to produce an aziridine product. In other aspects, the present invention provides heme enzymes including variants and fragments thereof that are capable of carrying out in vivo and in vitro olefin aziridination reactions. Expression vectors and host cells expressing the heme enzymes are also provided by the present invention.

### CROSS-REFERENCES TO RELATED APPLICATIONS

The present application claims priority to U.S. Provisional Patent Application No. 62/108,300, filed Jan. 27, 2015, and U.S. Provisional Patent Application No. 62/120,126, filed Feb. 24, 2015, the contents of which are hereby incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support under Grant No. N00014-11-1-0205 awarded by the Office of Naval Research. The government has certain rights in the invention.

### REFERENCE TO A SEQUENCE LISTING

The Sequence Listing written in file 086544-019120US-0966192_SequenceListing.txt, created on Apr. 11, 2016, 418,993 bytes, machine format IBM-PC, MS-Windows operating system, is hereby incorporated by reference in its entirety for all purposes.

### BACKGROUND OF THE INVENTION

Aziridines are 3 membered cyclic compounds comprising 2 carbons and a nitrogen that are often used as building blocks in various synthetic strategies. Traditional synthesis of aziridines can be achieved through various known methods; however, many of these method use caustic chemicals, harsh reaction conditions, and/or are unable to produce stereo-selective chiral aziridines.

Enzymes offer appealing alternatives to traditional chemical catalysts due to their ability to function in aqueous media at ambient temperature and pressure, as well as their ability to orient substrate binding for defined regio- and stereo-chemical outcomes. Indeed, the use of enzymes in synthetic chemistry to achieve otherwise difficult or low yielding chemical conversions is continuing to increase.

Although chemically attractive, enzymes are also known for their high substrate specificity and their catalytic fidelity. While this selectivity can be advantageous in some cases, it is also a significant synthetic limitation because each enzyme typically catalyzes only a specific chemical reaction. Despite these limitations, previous studies have shown that the native activity of enzymes can be modified or altered to catalyze non-natural or non-naturally occurring chemical conversions. Development of an enzyme capable of catalyzing an aziridination reaction could avoid using caustic chemicals, harsh reaction conditions, and could reliably produce stereo-selective chiral aziridines.

As such, there is a need in the art for novel reagents and catalytic schemes that are capable of creating an aziridine functional group with high yield, regioselectivity, and stereoselectivity, but without the toxicity and harsh reaction conditions associated with current approaches. The present invention addresses these and other needs.

### BRIEF SUMMARY OF THE INVENTION

In a first aspect, the invention provides a reaction mixture for producing an aziridination product. The reaction mixture includes an olefinic substrate, a nitrene precursor, and a heme enzyme.

In some embodiments, the olefinic substrate is represented by a structure of Formula I:

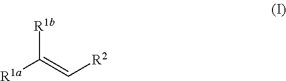

wherein:
R^{1a}, R^{1b}, and R² are independently selected from the group consisting of H, C₁₋₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl;
Y¹ is C₁₋₈alkylene;
each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen;
wherein each aryl contains between 6-14 carbon atoms, each heteroaryl group has from 5 to 8 ring atoms and from 1-3 heteroatoms selected from N, O and S, and each heterocyclyl group has from 1-3 heteroatoms selected from N, O and S.

In some instances, R^{1a}, R^{1b}, and R² are independently selected from the group consisting of H, C₁₋₁₈alkyl, aryl, heteroaryl, C₁₋₁₂cycloalkyl, and C₃₋₁₀heterocyclyl, and each R^{1a}, R^{1b}, and R² is optionally substituted with from 1 to 5 substituents independently selected from the group consisting of C₁₋₃alkyl, alkoxy, and halogen.

In some embodiments, the nitrene precursor has a structure according to Formula IIa or IIb:

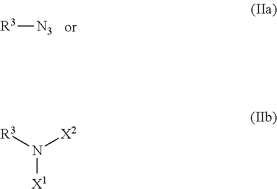

wherein:
R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c};
X¹ is independently selected from the group consisting of H and sodium, and
X² is independently selected from the group consisting of halogen, —SO₂R^{a}, —CO₂R^{b}, —PO₃R^{b}R^{c}, optionally X¹ and X² can be taken together to form iodinane;
R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl;
R^{b} and R^{c} are independently selected from the group consisting of C₁₋₈alkyl, C₃₋₁₂cycloalkyl, aryl, heteroaryl, and C₃₋₈heterocyclyl;
wherein within each R³, R^{a}, R^{b}, and R^{c} can be optionally substituted with from 1-5 R^{d} substituents;
each R^{d} is independently selected from the group consisting of C₁₋₃alkyl, halogen, and hydroxy; and
wherein each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

In some embodiments, the nitrene precursor has a structure selected from the group consisting of:

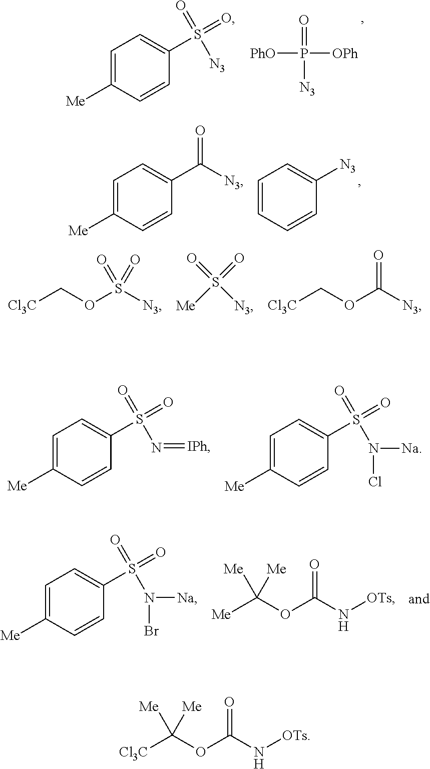

In some instances, the nitrene precursor is

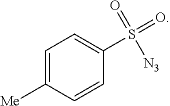 [SMILES: Cc1ccc(S(=O)(=O)N=[N+]=[N-])cc1]

In some embodiments, the aziridination product is produced in vitro.

In some embodiments, the reaction mixture further comprises a reducing agent. In some instances, the reducing agent is NADPH.

In some embodiments, the heme the heme enzyme is localized within a whole cell and the aziridination product is produced in vivo. In some instances, the whole cell is a bacterial cell or a yeast cell.

In some embodiments, the aziridination product is produced under anaerobic conditions.

In some embodiments, the heme enzyme is a variant thereof comprising a mutation at the axial position of the heme coordination site. In some instances, the heme enzyme comprises a serine mutation at the axial position of the heme coordination site.

In some embodiments, the heme enzyme is a cytochrome P450 enzyme or a variant thereof. In some instances, the cytochrome P450 enzyme is a P450 BM3 enzyme or a variant thereof.

In some embodiments, the P450 BM3 enzyme comprises an axial ligand mutation C400S and one or more mutations selected from the group consisting of V78, F87, P142, T175, A184, S226, H236, E252, I263, T268, A290, A328, L353, I366, L437, T438, and E442 relative to the amino acid sequence set forth in SEQ ID NO:1 (SEQ ID NO: 50). In some instances, the P450 BM3 enzyme comprises an axial ligand mutation C400S and mutations V78A, F87V, P142S, T175I, A184V, S226R, H236Q, E252G, I263F, T268A, A290V, A328V, L353V, I366V, L437V, T438S, and E442K relative to the amino acid sequence set forth in SEQ ID NO:1 (SEQ ID NO: 51).

In some embodiments, the P450 BM3 enzyme comprises an axial ligand mutation C400S and one or more mutations selected from the group consisting of L75, V78, F87, P142, T175, L181, A184, S226, H236, E252, I263, T268, A290, L353, I366, and E442 relative to the amino acid sequence set forth in SEQ ID NO:1 (SEQ ID NO: 52). In some instances, the P450 BM3 enzyme comprises an axial ligand mutation C400S and mutations L75A, V78A, F87V, P142S, T175I, L181A, A184V, S226R, H236Q, E252G, I263F, T268A, A290V, L353V, I366V, and E442K relative to the amino acid sequence set forth in SEQ ID NO:1 (SEQ ID NO: 53).

In some embodiments, the aziridination product is an aziridine compound according to Formula III:

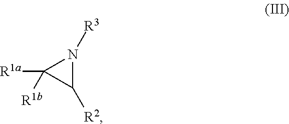

wherein
R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl;
Y¹ is C₁₋₈alkylene;
each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen;
R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c};
R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl;
R^{b} and R^{c} are independently selected from the group consisting of C₁₋₈alkyl, C₃₋₁₂cycloalkyl, aryl, heteroaryl, and C₃₋₈heterocyclyl;
wherein within each R³, R^{a}, R^{b}, and R^{c} can be optionally substituted with from 1-5 R^{d} substituents;
each R^{d} is independently selected from the group consisting of C₁₋₃alkyl, halogen, and hydroxy; and
wherein each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

In some instances, R^{1a} and R^{1b} are independently selected from the group consisting of H, C₁₋₈alkyl, aryl, heteroaryl, C₁₋₁₂cycloalkyl, and C₃₋₁₀heterocyclyl;
R² is selected from the group consisting of H and C₁₋₈ alkyl;
each R^{1a}, R^{1b}, and R² is optionally substituted with from 1 to 3 substituents independently selected from the group consisting of C₁₋₃alkyl, alkoxy, and halogen; and
R³ is selected from the group consisting of —SO₂R^{a}, —COR^{a}, —CO₂R^{b}, —PO₃R^{b}R^{c}, and —CONR^{b}R^{c},
R^{a} is independently selected from the group consisting of C₃₋₁₂cycloalkyl, aryl, heteroaryl, and C₃₋₈heterocyclyl;
R^{b} and R^{c} are independently selected from the group consisting of C₃₋₁₂cycloalkyl, aryl, heteroaryl, and C₃₋₈heterocyclyl;
wherein within each R³, R^{a}, R^{b}, and R^{c} can be optionally substituted with from 1-2 R^{d} substituents; and
each R^{d} is independently selected from the group consisting of C₁₋₃alkyl, halogen, and hydroxy.

In some embodiments, the aziridination product is an amido-alcohol compound according for Formula IIIa:

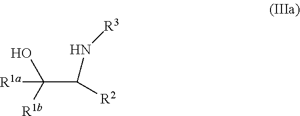

wherein R^{1a}, R^{1b}, R³, and R³ can be as defined above in Formula III.

In some embodiments, the reaction produces a plurality of aziridination products. In some instances, the plurality of aziridination products has a % ee_{S} of from about −99% to about 99%. In some instances, the plurality of aziridination products has a % ee_{S} of from about −81% to about 81%. In some instances, the plurality of aziridination products has a Z:E ratio of from about 1:99 to about 99:1.

In some aspects, the present invention provides a cytochrome P450 BM3 enzyme variant or fragment thereof that can a aziridinate an olefinic substrate comprising an axial ligand mutation C400S, mutations V78A, F87V, P142S, T175I, A184V, S226R, H236Q, E252G, I263F, T268A, A290V, L353V, I366V, T438S, and E442K, and at least one or more mutations at positions A328 and/or L437 relative to the amino acid sequence set forth in SEQ ID NO:1 (SEQ ID NO: 54). In some instances, the cytochrome P450 BM3 enzyme variant comprises an axial ligand mutation C400S and mutations V78A, F87V, P142S, T175I, A184V, S226R, H236Q, E252G, I263F, T268A, A290V, A328V, L353V, I366V, L437V, T438S, and E442K relative to the amino acid sequence set forth in SEQ ID NO: 1 (SEQ ID NO: 51).

In some embodiments, the cytochrome P450 BM3 enzyme variant produces a plurality of aziridination products with a % ee_{S} of at least about 75%. In some instances, the enzyme variant has a higher total turnover number (TTN) compared to the wild-type sequence. In some instances, the enzyme variant has a TTN greater than about 100.

In another aspect, the present invention provides a cytochrome P450 BM3 enzyme variant or fragment thereof that can aziridinate an olefinic substrate comprising an axial ligand mutation C400S, mutations L75A, V87A, F87V, P142S, T175I, L181A, A184V, S226R, H236Q, E252G, T268A, A290V, L353V, I366V, and E442K, and a mutation at position I263 relative to the amino acid sequence set forth in SEQ ID NO: 1. In some instances, the enzyme variant comprises an axial ligand mutation C400S and mutations L75A, V78A, F87V, P142S, T175I, L181A, A184V, S226R, H236Q, E252G, I263F, T268A, A290V, L353V, I366V, and E442K relative to the amino acid sequence set forth in SEQ ID NO: 1 (SEQ ID NO: 53).

In some embodiments, the cytochrome P450 BM3 enzyme variant produces a plurality of aziridination products with a % ee_{R} of at least about 75%. In some instances, the enzyme variant has a higher total turnover number (TTN) compared to the wild-type sequence. In some instances, the enzyme variant has a TTN greater than about 100.

In certain aspects, the present invention provides a method for producing an aziridination product, the method comprising:
(a) providing an olefinic substrate, a nitrene precursor, and a heme enzyme; and
(b) admixing the components of step (a) in a reaction for a time sufficient to produce an aziridination product.

In some embodiments, the method produces a plurality of aziridination products. In some instances, the plurality of aziridination products has a % ee_{S} of from about −90% to about 90%. In certain instances, the plurality of aziridination products has a % ee_{S} of from about −81% to about 81%. In some instances, the plurality of aziridination products has a Z:E ratio of from 1:99 to 99:1. In some instances, the aziridination reaction is at least 30% to at least 90% diastereoselective.

In some embodiments, the aziridination product is a compound according to Formula III:

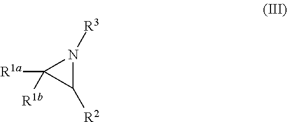

wherein R^{1a}, R^{1b}, R³, and R³ can be as defined above in Formula III.

In certain other embodiments, the aziridination product is a compound according to Formula IIIa:

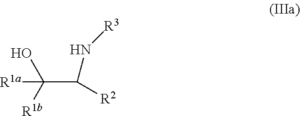

wherein R^{1a}, R^{1b}, R³, and R³ can be as defined above in Formula III.

Other objects, features, and advantages of the present invention will be apparent to one of skill in the art from the following detailed description and figures.

### BRIEF DESCRIPTION OF THE DRAWINGS

FIG. 1 shows a heme enzyme intermolecular nitrogen-atom transfer in accordance with an embodiment of the invention.

FIGS. 2A-B show HPLC 220 nm chromatograms of controls: FIG. 2A—Co-injection of 4-methoxystyrene (Sigma Aldrich) and an N-(p-Tolylsulfonyl)-2-(p-methoxyphenyl)aziridine synthetic standard, confirmed by NMR; FIG. 2B—Injection of the N-(p-Tolylsulfonyl)-2-(p-methoxyphenyl)aziridine synthetic standard alone.

FIGS. 3A-D show HPLC 220 nm chromatograms of P411-enzymatic reactions with 4-methoxystyrene and tosyl azide as substrates analyzed at different time points. Putative N-(p-Tolyl sulfonyl)-2-(p-methoxyphenyl)aziridine and amido-alcohol derivative ((N-(2-hydroxy-2-(4-methoxyphenyl)ethyl)-4-methylbenzenesulfonamide, 2) are marked with arrows.

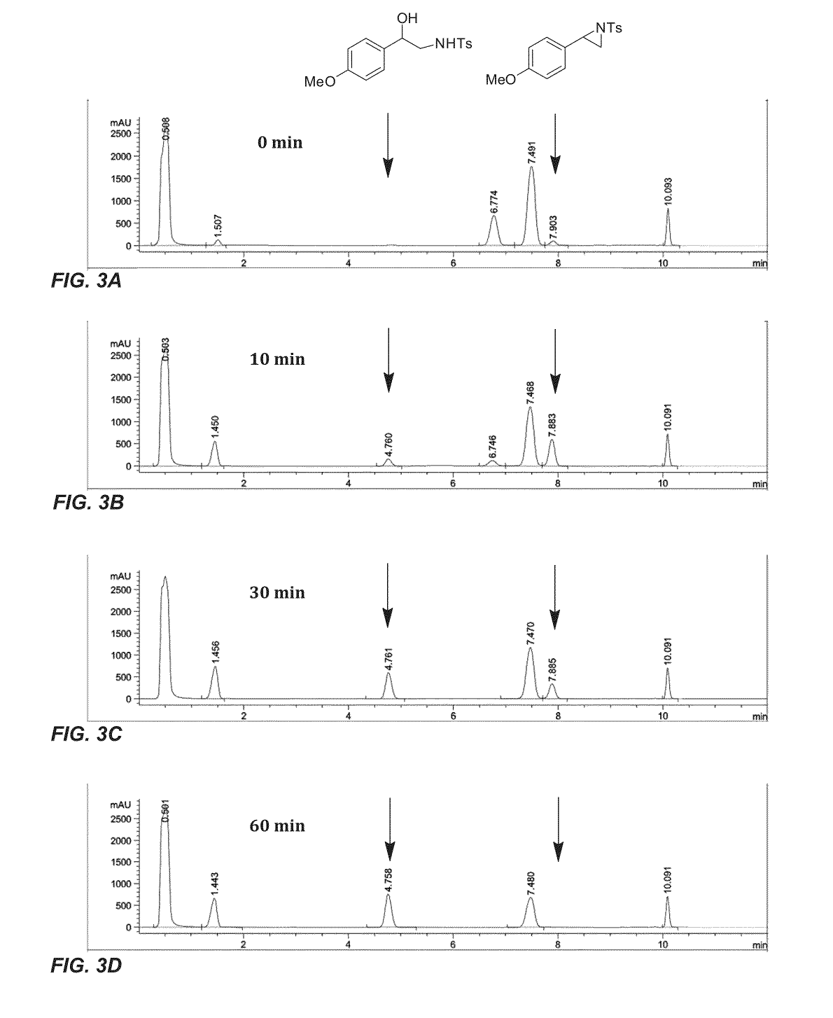

FIGS. 4A-D show HPLC 220 nm chromatograms of synthetic standard S1, synthesized as previously reported, in reaction conditions without P411 catalyst at several time points. Putative aziridine and amido-alcohol are marked with arrows, as in FIGS. 3A-D.

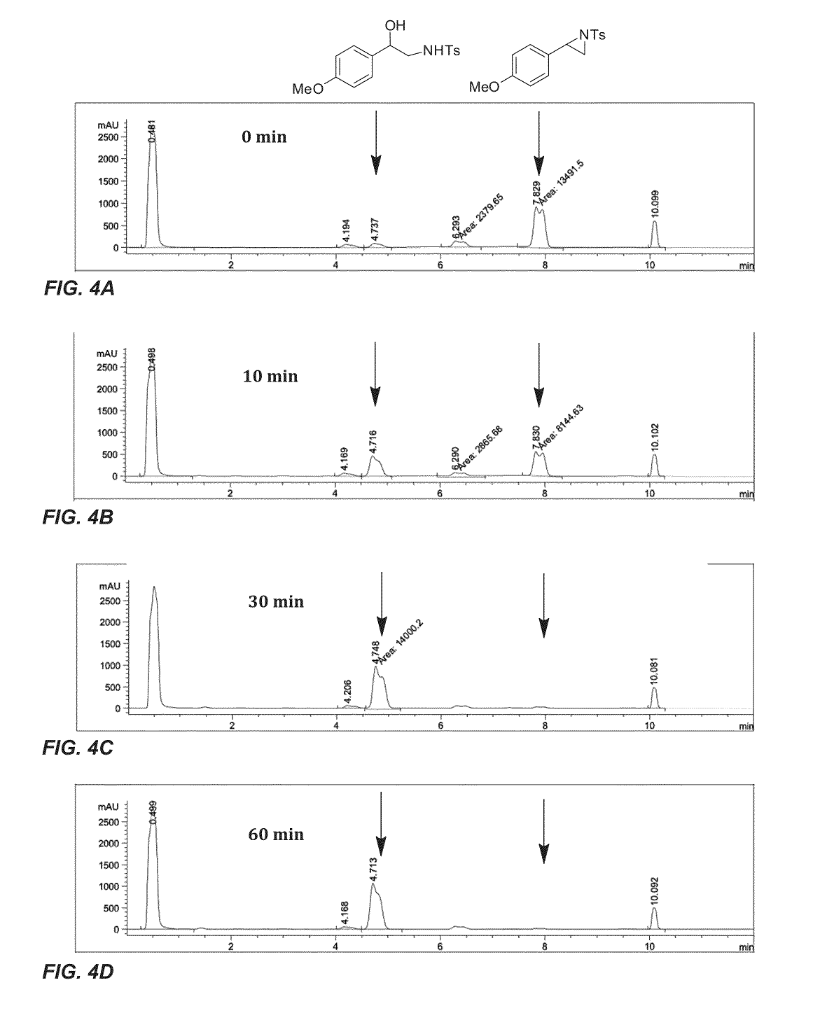

FIG. 5 shows a comparison of P-I263F productivity in vitro (purified protein) and in whole cells.

FIG. 6 shows initial rates of aziridination and azide reduction for engineered enzymes. Total turnover (TTN) values were determined using the same method as described for initial rates, with the exception that reactions were allowed to proceed for 4 hours in the anaerobic chamber.

FIGS. 7A-C show data used to determine initial rates for enzymes (A) P-I263F, (B) P-I263F-A328V, and (C) P-I263F-A328V-L437V. Diamonds represent concentrations of tosyl sulfonamide 7 and triangles represent concentrations of aziridine 4 for all plots.

FIG. 8 shows activity and selectivity of P-I263F-A328V-L437V with increased substrate loading. Reactions were performed with whole E. coli cells expressing P-I263F-A328V-L437V as described in the general methods, except substrate loading was increased to final concentrations of 7.5 mM tosyl azide and 15 mM olefin.

FIGS. 9A-B are an exemplary demonstration of how absolute stereochemistry can be defined for the products herein. The assignment of absolute stereochemistry of enzymatically produced aziridine 6 is assigned using chiral HPLC (Chiracel OJ, 30% isopropanol: 70% n-hexane, 210 nm). Top: Racemic synthetic aziridine 6, t_{R}=16.7 min and 21.0 min; Bottom: P-I263F-A328V-L437V produced aziridine 6, t_{R}=16.8 min.

### DETAILED DESCRIPTION OF THE INVENTION

### I. Introduction

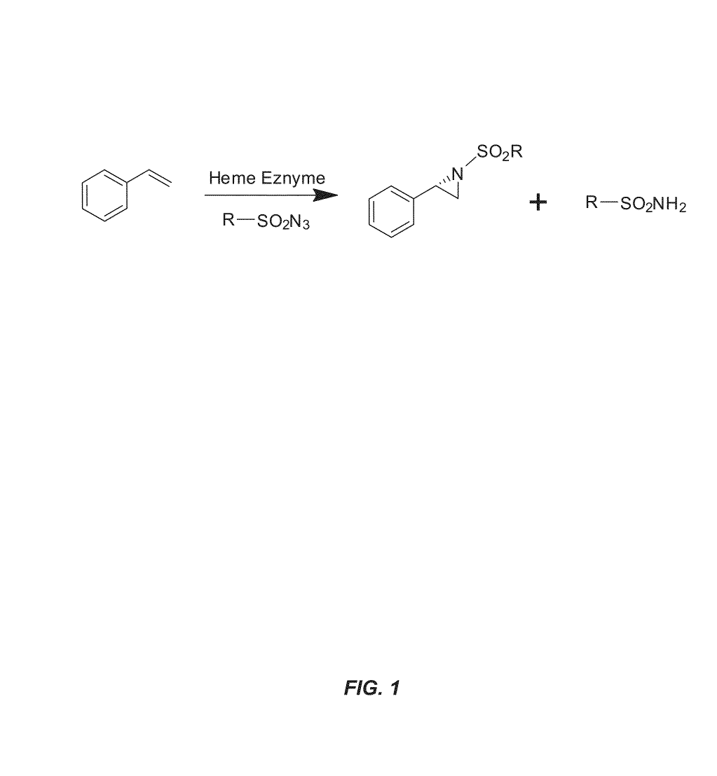

The present invention is based, in part, on the surprising discovery that heme enzymes can be used to catalyze the conversion of olefinic bonds to aziridination products in the presence of nitrene precursors. FIG. 1 illustrates an exemplary reaction where styrene is converted to an aziridination product. In some aspects, cytochrome P450 BM3 enzymes and variants thereof were identified as having an unexpectedly efficient ability to catalyze the formal transfer of nitrene equivalents from nitrene precursors to various olefinic substrates, thereby making aziridination products with high regioselectivity and/or stereoselectivity. In particular embodiments, the present inventors have discovered that variants of P450 BM3 with at least one or more amino acid mutations such as an axial ligand C400S, mutations V78A, F87V, P142S, T175I, A184V, S226R, H236Q, E252G, I263F, T268A, A290V, L353V, I366V, T438S, and E442K, and at least one or more mutations at positions A328 and/or L437 (SEQ ID NO: 54) can catalyze aziridination reactions efficiently, displaying increased total turnover numbers (TTN) and demonstrating highly regio- and enantioselective product formation compared to wild-type enzymes.

Aziridination reactions can be performed by the heme enzymes described herein in vitro or in vivo, where the heme enzyme is localized within a whole cell. In some embodiments, the heme enzyme described herein can catalyze the aziridination reaction in vivo, providing over 500 total turnovers with high stereoselectivity and yield.

The disclosure herein highlights the utility of enzymes in catalyzing new types of reactions. The ability to genetically encode catalysts for formal nitrene transfers opens up new biosynthetic pathways to amines and expands the scope of transformations accessible to biocatalysis.

### II. Definitions

The following definitions and abbreviations are to be used for the interpretation of the invention. The term “invention” or “present invention” as used herein is a non-limiting term and is not intended to refer to any single embodiment but encompasses all possible embodiments.

As used herein and in the appended claims, the singular forms “a,” “and,” and “the” include plural referents unless the context clearly dictates otherwise. Thus, for example, reference to “a species” includes a plurality of such species and reference to “the enzyme” includes reference to one or more enzymes and equivalents thereof, and so forth.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

“About” and “around,” as used herein to modify a numerical value, indicate a defined range around that value. If “X” were the value, “about X” or “around X” would generally indicate a value from 0.95X to 1.05X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.” When the quantity “X” only includes whole-integer values (e.g., “X carbons”), “about X” or “around X” indicates from (X−1) to (X+1). In such cases, “about X” or “around X” specifically indicates at least the values X, X−1, and X+1.

The term “aziridination (enzyme) catalyst” or “enzyme with aziridination activity” refers to any and all chemical processes catalyzed by enzymes, by which substrates containing at least one carbon-carbon double bond can be converted into an aziridination product by using nitrene precursors.

The terms “engineered heme enzyme” and “heme enzyme variant” include any heme enzyme comprising at least one amino acid mutation with respect to wild-type and also include any chimeric protein comprising recombined sequences or blocks of amino acids from two, three, or more different heme enzymes.

The terms “engineered cytochrome P450” and “cytochrome P450 variant” include any cytochrome P450 enzyme comprising at least one amino acid mutation with respect to wild-type and also include any chimeric protein comprising recombined sequences or blocks of amino acids from two, three, or more different cytochrome P450 enzymes.

The term “whole cell catalyst” includes microbial cells expressing at least one engineered heme enzyme, wherein the whole cell catalyst displays aziridination activity.

As used herein, the terms “porphyrin” and “metal-substituted porphyrins” include any porphyrin that can be bound by a heme enzyme or variant thereof. In particular embodiments, these porphyrins may contain metals including, but not limited to, Fe, Mn, Co, Cu, Rh, and Ru.

The term “heme” or “heme domain” as used herein refers to an amino acid sequence within an enzyme, which is capable of binding an iron-complexing structure such as a porphyrin. Compounds of iron are typically complexed in a porphyrin (tetrapyrrole) ring that may differ in side chain composition. Heme groups can be the prosthetic groups of cytochromes and are found in most oxygen carrier proteins. Exemplary heme domains include that of P450 BM3 as well as truncated or mutated versions of these that retain the capability to bind the iron-complexing structure. A skilled person can identify the heme domain of a specific protein using methods known in the art.

The terms “nitrene equivalent” and “nitrene precursor” include molecules that can be decomposed in the presence of metal (or enzyme) catalysts to structures that contain at least one nitrogen with only 5 valence shell electrons and that can be transferred to C═C bonds to form aziridines. Nitrene precursors of the present invention include, but are not limited to, sulfonyl azides, carbonyl azides, aryl azides, azidoformates, phosphoryl azides, azide phosphonates, iminoiodanes, or haloamine derivatives.

The terms “nitrene transfer” and “formal nitrene transfer” as used herein include any chemical transformation where nitrene equivalents are added to C═C bonds.

As used herein, the terms “microbial,” “microbial organism” and “microorganism” include any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. Also included are cell cultures of any species that can be cultured for the production of a chemical.

As used herein, the term “non-naturally occurring”, when used in reference to a microbial organism or enzyme activity of the invention, is intended to mean that the microbial organism or enzyme has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary non-naturally occurring microbial organism or enzyme activity includes the aziridination activity described below.

As used herein, the term “anaerobic”, when used in reference to a reaction, culture or growth condition, is intended to mean that the concentration of oxygen is less than about 25 μM, preferably less than about 5 μM, and even more preferably less than 1 μM. The term is also intended to include sealed chambers of liquid or solid medium maintained with an atmosphere of less than about 1% oxygen. Preferably, anaerobic conditions are achieved by sparging a reaction mixture with an inert gas such as nitrogen or argon.

As used herein, the term “exogenous” is intended to mean that the referenced molecule or the referenced activity is introduced into the host microbial organism. The term as it is used in reference to expression of an encoding nucleic acid refers to the introduction of the encoding nucleic acid in an expressible form into the microbial organism. When used in reference to a biosynthetic activity, the term refers to an activity that is introduced into the host reference organism.

The term “heterologous” as used herein with reference to molecules, and in particular enzymes and polynucleotides, indicates molecules that are expressed in an organism other than the organism from which they originated or are found in nature, independently of the level of expression that can be lower, equal or higher than the level of expression of the molecule in the native microorganism.

On the other hand, the term “native” or “endogenous” as used herein with reference to molecules, and in particular enzymes and polynucleotides, indicates molecules that are expressed in the organism in which they originated or are found in nature, independently of the level of expression that can be lower equal or higher than the level of expression of the molecule in the native microorganism. It is understood that expression of native enzymes or polynucleotides may be modified in recombinant microorganisms.

The term “homolog,” as used herein with respect to an original enzyme or gene of a first family or species, refers to distinct enzymes or genes of a second family or species which are determined by functional, structural or genomic analyses to be an enzyme or gene of the second family or species which corresponds to the original enzyme or gene of the first family or species. Homologs most often have functional, structural, or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the amino acid sequence encoded by a gene has a similar amino acid sequence to that of the second gene. Alternatively, a protein has homology to a second protein if the two proteins have “similar” amino acid sequences. Thus, the term “homologous proteins” is intended to mean that the two proteins have similar amino acid sequences. In particular embodiments, the homology between two proteins is indicative of its shared ancestry, related by evolution.

The terms “analog” and “analogous” include nucleic acid or protein sequences or protein structures that are related to one another in function only and are not from common descent or do not share a common ancestral sequence. Analogs may differ in sequence but may share a similar structure, due to convergent evolution. For example, two enzymes are analogs or analogous if the enzymes catalyze the same reaction of conversion of a substrate to a product, are unrelated in sequence, and irrespective of whether the two enzymes are related in structure.

As used herein, the term “electron withdrawing group” refers to an atom or substituent that has an ability to acquire electron density from an olefin or other atoms or substituents. An “electron withdrawing group” is capable of withdrawing electron density relative to that of hydrogen if the hydrogen atom occupied the same position on the molecule. The term “electron withdrawing group” is well understood by those of skill in the art and is discussed, for example, in Advanced Organic Chemistry by J. March, John Wiley & Sons, New York, N.Y., (1985). Examples of electron withdrawing groups include, but are not limited to, halo (e.g., fluorine, chlorine, bromine, iodine), nitro, carboxy, amido, acyl, cyano, aryl, heteroaryl, —OC(A)₃, —C(A)₃, —C(A)₂-O-C(A′)₃, —(CO)-Q, —SO₂—C(A)₃, —SO₂-aryl, —C(NQ)Q, —CH═C(Q)₂, and —C≡C-Q; in which each A and A′ is independently H, halo, —CN, —NO₂, —OH, or C₁₋₄ alkyl optionally substituted with 1-3 halo, —OH, or NO₂; and Q is selected from H, —OH, and alkyl optionally substituted with 1-3 halo, —OH, —O-alkyl, or —O-cycloalkyl.

The term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C_{5- 6}. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

The term “alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

As used herein, the terms “halo” and “halogen” refer to fluorine, chlorine, bromine and iodine.

The term “heteroalkyl” refers to an alkyl group of any suitable length and having from 1 to 3 heteroatoms such as N, O and S. Additional heteroatoms can also be useful, including, but not limited to, B, Al, Si and P. The heteroatoms can also be oxidized, such as, but not limited to, —SO— and —SO₂—. For example, heteroalkyl can include ethers, thioethers and alkyl-amines. The heteroatom portion of the heteroalkyl can be the connecting atom, or be inserted between two carbon atoms.

The term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

The term “heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, S═O and SO₂ (two double bonded oxygens). Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. Heteroaryl groups can be linked via any position on the ring.

The term “cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

The term “iodinane” refers to the chemical substituent

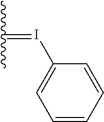 [SMILES: *=Ic1ccccc1]

where the ‘wavy line’ represents the point of attachment to the remainder of the molecule.

Any compound or formula disclosed herein that does not define the chirality of a chiral carbon can be a racemic mixture or may possess an enantiomeric excess of R or S isomers. For example, compounds represented by Formula III, shown below, may possess, 0, 1, or 2 chiral carbons depending on the identities of R^{1a}, R^{1b}, and R². Each chiral center may be racemic or may be of a particular enantiomeric excess. A person of skill in the art will also recognize that instances of two chiral carbons within an single aziridine ring can produce two cis and two trans isomers.

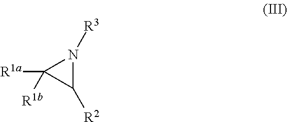

### III. Description of the Embodiments

In a first aspect, the invention provides a reaction mixture for producing an aziridination product. The reaction mixture contains an olefinic substrate, a nitrene precursor, and a heme enzyme.

In other aspects, the present invention provides heme enzymes including variants thereof that are capable of carrying out the aziridination reactions described herein. Expression vectors and host cells expressing the heme enzymes are also provided by the present invention.

In still other aspects, the present invention provides methods for producing an aziridination product. In certain aspects, the present invention provides a method for producing an aziridination product, the method comprising:
(a) providing an olefinic substrate, a nitrene precursor, and a heme enzyme; and
(b) admixing the components of step (a) in a reaction for a time sufficient to produce an aziridination product.

The following sections provide a description of exemplary and preferred embodiments including heme enzymes, expression vectors, host cells, aziridination products such as, e.g., compounds comprising an aziridine functional group, starting materials such as, e.g., olefinic substrates and nitrene precursors, and characteristics and reaction conditions for the in vitro and in vivo aziridination reactions described herein.

A. Heme Enzymes

The terms “heme enzyme” and “heme protein” are used herein to include any member of a group of proteins containing heme as a prosthetic group. Non-limiting examples of heme enzymes include globins, cytochromes, oxidoreductases, any other protein containing a heme as a prosthetic group, and combinations thereof. Heme-containing globins include, but are not limited to, hemoglobin, myoglobin, and combinations thereof. Heme-containing cytochromes include, but are not limited to, cytochrome P450, cytochrome b, cytochrome c1, cytochrome c, and combinations thereof. Heme-containing oxidoreductases include, but are not limited to, a catalase, an oxidase, an oxygenase, a haloperoxidase, a peroxidase, and combinations thereof.

In certain aspects, the present invention provides compositions comprising one or more heme enzymes that catalyze the conversion of olefinic substrates to aziridination products. In particular embodiments, the present invention provides heme enzyme variants comprising at least one or more amino acid mutations therein that catalyze the formal transfer of a nitrene equivalent to an olefinic substrate, making aziridination products with high stereoselectivity. In preferred embodiments, the heme enzyme variants of the present invention have the ability to catalyze aziridination reactions efficiently, display increased total turnover numbers, and/or demonstrate highly regio- and/or enantioselective product formation compared to the corresponding wild-type enzymes.

In some embodiments, the enzyme is a heme enzyme or a variant thereof. In certain instances, the heme enzymes are metal-substituted heme enzymes containing protoporphyrin IX or other porphyrin molecules containing metals other than iron, including, but not limited to, cobalt, rhodium, copper, ruthenium, and manganese, which are active aziridination catalysts.

In some embodiments, the heme enzyme is a member of one of the enzyme classes set forth in Table A. In other embodiments, the heme enzyme is a variant or homolog of a member of one of the enzyme classes set forth in Table A. In yet other embodiments, the heme enzyme comprises or consists of the heme domain of a member of one of the enzyme classes set forth in Table A or a fragment thereof (e.g., a truncated heme domain) that is capable of carrying out the aziridination reactions described herein.

TABLE A
Heme enzymes identified by their enzyme classification
number (EC number) and classification name.

| EC Number | Name |
|---|---|
| 1.1.2.3 | L-lactate dehydrogenase |
| 1.1.2.6 | polyvinyl alcohol dehydrogenase (cytochrome) |
| 1.1.2.7 | methanol dehydrogenase (cytochrome c) |
| 1.1.5.5 | alcohol dehydrogenase (quinone) |
| 1.1.5.6 | formate dehydrogenase-N: |
| 1.1.9.1 | alcohol dehydrogenase (azurin): |
| 1.1.99.3 | gluconate 2-dehydrogenase (acceptor) |
| 1.1.99.11 | fructose 5-dehydrogenase |
| 1.1.99.18 | cellobiose dehydrogenase (acceptor) |
| 1.1.99.20 | alkan-1-ol dehydrogenase (acceptor) |
| 1.2.1.70 | glutamyl-tRNA reductase |
| 1.2.3.7 | indole-3-acetaldehyde oxidase |
| 1.2.99.3 | aldehyde dehydrogenase (pyrroloquinoline-quinone) |
| 1.3.1.6 | fumarate reductase (NADH): |
| 1.3.5.1 | succinate dehydrogenase (ubiquinone) |
| 1.3.5.4 | fumarate reductase (menaquinone) |
| 1.3.99.1 | succinate dehydrogenase |
| 1.4.9.1 | methylamine dehydrogenase (amicyanin) |
| 1.4.9.2. | aralkylamine dehydrogenase (azurin) |
| 1.5.1.20 | methylenetetrahydrofolate reductase [NAD(P)H] |
| 1.5.99.6 | spermidine dehydrogenase |
| 1.6.3.1 | NAD(P)H oxidase |
| 1.7.1.1 | nitrate reductase (NADH) |
| 1.7.1.2 | Nitrate reductase [NAD(P)H] |
| 1.7.1.3 | nitrate reductase (NADPH) |
| 1.7.1.4 | nitrite reductase [NAD(P)H] |
| 1.7.1.14 | nitric oxide reductase ]NAD(P), nitrous oxide-forming] |
| 1.7.2.1 | nitrite reductase (NO-forming) |
| 1.7.2.2 | nitrite reductase (cytochrome; ammonia-forming) |
| 1.7.2.3 | trimethylamine-N-oxide reductase (cytochrome c) |
| 1.7.2.5 | nitric oxide reductase (cytochrome c) |
| 1.7.2.6 | hydroxylamine dehydrogenase |
| 1.7.3.6 | hydroxylamine oxidase (cytochrome) |
| 1.7.5.1 | nitrate reductase (quinone) |
| 1.7.5.2 | nitric oxide reductase (menaquinol) |
| 1.7.6.1 | nitrite dismutase |
| 1.7.7.1 | ferredoxin-nitrite reductase |
| 1.7.7.2 | ferredoxin-nitrate reductase |
| 1.7.99.4 | nitrate reductase |
| 1.7.99.8 | hydrazine oxidoreductase |
| 1.8.1.2 | sulfite reductase (NADPH) |
| 1.8.2.1 | sulfite dehydrogenase |
| 1.8.2.2 | thiosulfate dehydrogenase |
| 1.8.2.3 | sulfide-cytochrome-c reductase (flavocytochrome c) |
| 1.8.2.4 | dimethyl sulfide:cytochrome c2 reductase |
| 1.8.3.1 | sulfite oxidase |
| 1.8.7.1 | sulfite reductase (ferredoxin) |
| 1.8.98.1 | CoB-CoM heterodisulfide reductase |
| 1.8.99.1 | sulfite reductase |
| 1.8.99.2 | adenylyl-sulfate reductase |
| 1.8.99.3 | hydrogensulfite reductase |
| 1.9.3.1 | cytochrome-c oxidase |
| 1.9.6.1 | nitrate reductase (cytochrome) |
| 1.10.2.2 | ubiquinol-cytochrome-c reductase |
| 1.10.3.1 | catechol oxidase |
| 1.10.3.B1 | caldariellaquinol oxidase (H+-transporting) |
| 1.10.3.3 | L-ascothate oxidase |
| 1.10.3.9 | photosystem II |
| 1.10.3.10 | ubiquinol oxidase (H+-transporting) |
| 1.10.3.11 | ubiquinol oxidase |
| 1.10.3.12 | menaquinol oxidase (H+-transporting) |
| 1.10.9.1 | plastoquinol-plastocyanin reductase |
| 1.11.1.5 | cytochrome-c peroxidase |
| 1.11.1.6 | catalase |
| 1.11.1.7 | peroxidase |
| 1.11.1.B2 | chloride peroxidase (vanadium-containing) |
| 1.11.1.B7 | bromide peroxidase (heme-containing) |
| 1.11.1.8 | iodide peroxidase |
| 1.11.1.10 | chloride peroxidase |
| 1.11.1.11 | L-ascothate peroxidase |
| 1.11.1.13 | manganese peroxidase |
| 1.11.1.14 | lignin peroxidase |
| 1.11.1.16 | versatile peroxidase |
| 1.11.1.19 | dye decolorizing peroxidase |
| 1.11.1.21 | catalase-peroxidase |
| 1.11.2.1 | unspecific peroxygenase |
| 1.11.2.2 | myeloperoxidase |
| 1.11.2.3 | plant seed peroxygenase |
| 1.11.2.4 | fatty-acid peroxygenase |
| 1.12.2.1 | cytochrome-c3 hydrogenase |
| 1.12.5.1 | hydrogen:quinone oxidoreductase |
| 1.12.99.6 | hydrogenase (acceptor) |
| 1.13.11.9 | 2,5-dihydroxypyridine 5,6-dioxygenase |
| 1.13.11.11 | tryptophan 2,3-dioxygenase |
| 1.13.11.49 | chlorite O2-lyase |
| 1.13.11.50 | acetylacetone-cleaving enzyme |
| 1.13.11.52 | indoleamine 2,3-dioxygenase |
| 1.13.11.60 | linoleate 8R-lipoxygenase |
| 1.13.99.3 | tryptophan 21-dioxygenase |
| 1.14.11.9 | flavanone 3-dioxygenase |
| 1.14.12.17 | nitric oxide dioxygenase |
| 1.14.13.39 | nitric-oxide synthase (NADPH dependent) |
| 1.14.13.17 | cholesterol 7alpha-monooxygenase |
| 1.14.13.41 | tyrosine N-monooxygenase |
| 1.14.13.70 | sterol 14alpha-demethylase |
| 1.14.13.71 | N-methylcoclaurine 3′-monooxygenase |
| 1.14.13.81 | magnesium-protoporphyrin IX monomethyl ester |
| | (oxidative) cyclase |
| 1.14.13.86 | 2-hydroxyisoflavanone synthase |
| 1.14.13.98 | cholesterol 24-hydroxylase |
| 1.14.13.119 | 5-epiaristolochene 1,3-dihydroxylase |
| 1.14.13.126 | vitamin D3 24-hydroxylase |
| 1.14.13.129 | beta-carotene 3-hydroxylase |
| 1.14.13.141 | cholest-4-en-3-one 26-monooxygenase |
| 1.14.13.142 | 3-ketosteroid 9alpha-monooxygenase |
| 1.14.13.151 | linalool 8-monooxygenase |
| 1.14.13.156 | 1,8-cineole 2-endo-monooxygenase |
| 1.14.13.159 | vitamin D 25-hydroxylase |
| 1.14.14.1 | unspecific monooxygenase |
| 1.14.15.1 | camphor 5-monooxygenase |
| 1.14.15.6 | cholesterol monooxygenase (side-chain-cleaving) |
| 1.14.15.8 | steroid 15beta-monooxygenase |
| 1.14.15.9 | spheroidene monooxygenase |
| 1.14.18.1 | tyrosinase |
| 1.14.19.1 | stearoyl-CoA 9-desaturase |
| 1.14.19.3 | linoleoyl-CoA desaturase |
| 1.14.21.7 | biflaviolin synthase |
| 1.14.99.1 | prostaglandin-endoperoxide synthase |
| 1.14.99.3 | heme oxygenase |
| 1.14.99.9 | steroid 17alpha-monooxygenase |
| 1.14.99.10 | steroid 21-monooxygenase |
| 1.14.99.15 | 4-methoxybenzoate monooxygenase (O-demethylating) |
| 1.14.99.45 | carotene epsilon-monooxygenase |
| 1.16.5.1 | ascorbate ferrireductase (transmembrane) |
| 1.16.9.1 | iron:rusticyanin reductase |
| 1.17.1.4 | xanthine dehydrogenase |
| 1.17.2.2 | lupanine 17-hydroxylase (cytochrome c) |
| 1.17.99.1 | 4-methylphenol dehydrogenase (hydroxylating) |
| 1.17.99.2 | ethylbenzene hydroxylase |
| 1.97.1.1 | chlorate reductase |
| 1.97.1.9 | selenate reductase |
| 2.7.7.65 | diguanylate cyclase |
| 2.7.13.3 | histidine kinase |
| 3.1.4.52 | cyclic-guanylate-specific phosphodiesterase |
| 4.2.1.B9 | colneleic acid/etheroleic acid synthase |
| 4.2.1.22 | Cystathionine beta-synthase |
| 4.2.1.92 | hydroperoxide dehydratase |
| 4.2.1.212 | colneleate synthase |
| 4.3.1.26 | chromopyrrolate synthase |
| 4.6.1.2 | guanylate cyclase |
| 4.99.1.3 | sirohydrochlorin cobaltochelatase |
| 4.99.1.5 | aliphatic aldoxime dehydratase |
| 4.99.1.7 | phenylacetaldoxime dehydratase |
| 5.3.99.3 | prostaglandin-E synthase |
| 5.3.99.4 | prostaglandin-I synthase |
| 5.3.99.5 | Thromboxane-A synthase |
| 5.4.4.5 | 9,12-octadecadienoate 8-hydroperoxide 8R-isomerase |
| 5.4.4.6 | 9,12-octadecadienoate 8-hydroperoxide 8S-isomerase |
| 6.6.1.2 | cobaltochelatase |

In some embodiments, the heme enzyme is a variant or a fragment thereof (e.g., a truncated variant containing the heme domain) comprising at least one mutation such as, e.g., a mutation at the axial position of the heme coordination site. In some instances, the mutation is a substitution of the native residue with Ala, Asp, Arg, Asn, Cys, Glu, Gln, Gly, His, Ile, Lys, Leu, Met, Phe, Pro, Ser, Thr, Trp, Tyr, or Val at the axial position. In certain instances, the mutation is a substitution of Cys with any other amino acid such as Ser at the axial position.

In certain embodiments, the in vitro methods for producing an aziridination product comprise providing a heme enzyme, variant, or homolog thereof with a reducing agent such as NADPH or a dithionite salt (e.g., Na₂S₂O₄). In certain other embodiments, the in vivo methods for producing an aziridination product comprise providing whole cells such as E. coli cells expressing a heme enzyme, variant, or homolog thereof.

In some embodiments, the heme enzyme, variant, or homolog thereof is recombinantly expressed and optionally isolated and/or purified for carrying out the in vitro aziridination reactions of the present invention. In other embodiments, the heme enzyme, variant, or homolog thereof is expressed in whole cells such as E. coli cells, and these cells are used for carrying out the in vivo aziridination reactions of the present invention.

In certain embodiments, the heme enzyme, variant, or homolog thereof comprises or consists of the same number of amino acid residues as the wild-type enzyme (e.g., a full-length polypeptide). In some instances, the heme enzyme, variant, or homolog thereof comprises or consists of an amino acid sequence without the start methionine (e.g., P450 BM3 amino acid sequence set forth in SEQ ID NO:1). In other embodiments, the heme enzyme comprises or consists of a heme domain fused to a reductase domain. In yet other embodiments, the heme enzyme does not contain a reductase domain, e.g., the heme enzyme contains a heme domain only or a fragment thereof such as a truncated heme domain.

In some embodiments, the heme enzyme, variant, or homolog thereof has an enhanced nitrene insertion activity and/or nitrene transfer activity of about 1.5, 2, 3, 4, 5, 6, 7, 8, 9, 10, 15, 20, 25, 30, 35, 40, 45, 50, 55, 60, 65, 70, 75, 80, 85, 90, 95, or 100 fold compared to the corresponding wild-type heme enzyme.

In some embodiments, the heme enzyme, variant, or homolog thereof has a resting state reduction potential higher than that of NADH or NADPH.

In particular embodiments, the heme enzyme comprises a cyctochrome P450 enzyme. Cytochrome P450 enzymes constitute a large superfamily of heme-thiolate proteins involved in the metabolism of a wide variety of both exogenous and endogenous compounds. Usually, they act as the terminal oxidase in multicomponent electron transfer chains, such as P450-containing monooxygenase systems. Members of the cytochrome P450 enzyme family catalyze myriad oxidative transformations, including, e.g., hydroxylation, epoxidation, oxidative ring coupling, heteroatom release, and heteroatom oxygenation (E. M. Isin et al., Biochim. Biophys. Acta 1770, 314 (2007)). The active site of these enzymes contains an FeIII-protoporphyrin IX cofactor (heme) ligated proximally by a conserved cysteine thiolate (M. T. Green, Current Opinion in Chemical Biology 13, 84 (2009)).

One skilled in the art will appreciate that the cytochrome P450 enzyme superfamily has been compiled in various databases, including, but not limited to, the P450 homepage (available at http://drnelson.uthsc.edu/CytochromeP450.html; see also, D. R. Nelson, Hum. Genomics 4, 59 (2009)), the cytochrome P450 enzyme engineering database (available at http://www.cyped.uni-stuttgart.de/cgi-bin/CYPED5/index.pl; see also, D. Sirim et al., BMC Biochem 10, 27 (2009)), and the SuperCyp database (available at http://bioinformatics.charite.de/supercyp/; see also, S. Preissner et al., Nucleic Acids Res. 38, D237 (2010)), the disclosures of which are incorporated herein by reference in their entirety for all purposes.

In certain embodiments, the cytochrome P450 enzymes of the invention are members of one of the classes shown in Table B (see, http://www.icgeb.org/˜p450srv/P450enzymes.html, the disclosure of which is incorporated herein by reference in its entirety for all purposes).

TABLE B
Cytochrome P450 enzymes classified by their EC number,
recommended name, and family/gene name.

| EC | Recommended name | Family/gene |
|---|---|---|
| 1.3.3.9 | secologanin synthase | CYP72A1 |
| 1.14.13.11 | trans-cinnamate 4-monooxygenase | CYP73 |
| 1.14.13.12 | benzoate 4-monooxygenase | CYP53 |
| 1.14.13.13 | calcidiol 1-monooxygenase | CYP27 |
| 1.14.13.15 | cholestanetriol 26-monooxygenase | CYP27 |
| 1.14.13.17 | cholesterol 7x-monooxygenase | CYP7 |
| 1.14.13.21 | flavonoid 3′-monooxygenase | CYP75 |
| 1.14.13.28 | 3,9-dihydroxypterocarpan 6a-monooxygenase | CYP93A1 |
| 1.14.13.30 | leukotriene-B₄ 20-monooxygenase | CYP4F |
| 1.14.13.37 | methyltetrahydroprotoberberine 14-monooxygenase | CYP93A1 |
| 1.14.13.41 | tyrosine N-monooxygenase | CYP79 |
| 1.14.13.42 | hydroxyphenylacetonitrile 2-monooxygenase | — |
| 1.14.13.47 | (-)-limonene 3-monooxygenase | — |
| 1.14.13.48 | (-)-limonene 6-monooxygenase | — |
| 1.14.13.49 | (-)-limonene 7-monooxygenase | — |
| 1.14.13.52 | isoflavone 3′-hydroxylase | — |
| 1.14.13.53 | isoflavone 2′-hydroxylase | — |
| 1.14.13.55 | protopine 6-monooxygenase | — |
| 1.14.13.56 | dihydrosanguinarine 10-monooxygenase | — |
| 1.14.13.57 | dihydrochelirubine 12-monooxygenase | — |
| 1.14.13.60 | 27-hydroxycholesterol 7x-monooxygenase | — |
| 1.14.13.70 | sterol 14-demethylase | CYP51 |
| 1.14.13.71 | N-methylcoclaurine 3′-monooxygenase | CYP80B1 |
| 1.14.13.73 | tabersonine 16-hydroxylase | CYP71D12 |
| 1.14.13.74 | 7-deoxyloganin 7-hydroxylase | — |
| 1.14.13.75 | vinorine hydroxylase | — |
| 1.14.13.76 | taxane 10β-hydroxylase | CYP725A1 |
| 1.14.13.77 | taxane 13α-hydroxylase | CYP725A2 |
| 1.14.13.78 | ent-kaurene oxidase | CYP701 |
| 1.14.13.79 | ent-kaurenoic acid oxidase | CYP88A |
| 1.14.14.1 | unspecific monooxygenase | multiple |
| 1.14.15.1 | camphor 5-monooxygenase | CYP101 |
| 1.14.15.3 | alkane 1-monooxygenase | CYP4A |
| 1.14.15.4 | steroid 11β-monooxygenase | CYP11B |
| 1.14.15.5 | corticosterone 18-monooxygenase | CYP11B |
| 1.14.15.6 | cholesterol monooxygenase (side-chain-cleaving) | CYP11A |
| 1.14.21.1 | (S)-stylopine synthase | — |
| 1.14.21.2 | (S)-cheilanthifoline synthase | — |
| 1.14.21.3 | berbamunine synthase | CYP80 |
| 1.14.21.4 | salutaridine synthase | — |
| 1.14.21.5 | (S)-canadine synthase | — |
| 1.14.99.9 | steroid 17x-monooxygenase | CYP17 |
| 1.14.99.10 | steroid 21-monooxygenase | CYP21 |
| 1.14.99.22 | ecdysone 20-monooxygenase | — |
| 1.14.99.28 | linalool 8-monooxygenase | CYP111 |
| 4.2.1.92 | hydroperoxide dehydratase | CYP74 |
| 5.3.99.4 | prostaglandin-I synthase | CYP8 |
| 5.3.99.5 | thromboxane-A synthase | CYP5 |

Table C below lists additional cyctochrome P450 enzymes that are suitable for use in the aziridination reactions of the present invention. The accession numbers in Table C are incorporated herein by reference in their entirety for all purposes. The cytochrome P450 gene and/or protein sequences disclosed in the following patent documents are hereby incorporated by reference in their entirety for all purposes: WO 2013/076258; CN 103160521; CN 103223219; KR 2013081394; JP 5222410; WO 2013/073775; WO 2013/054890; WO 2013/048898; WO 2013/031975; WO 2013/064411; U.S. Pat. No. 8,361,769; WO 2012/150326, CN 102747053; CN 102747052; JP 2012170409; WO 2013/115484; CN 103223219; KR 2013081394; CN 103194461; JP 5222410; WO 2013/086499; WO 2013/076258; WO 2013/073775; WO 2013/064411; WO 2013/054890; WO 2013/031975; U.S. Pat. No. 8,361,769; WO 2012/156976; WO 2012/150326; CN 102747053; CN 102747052; US 20120258938; JP 2012170409; CN 102399796; JP 2012055274; WO 2012/029914; WO 2012/028709; WO 2011/154523; JP 2011234631; WO 2011/121456; EP 2366782; WO 2011/105241; CN 102154234; WO 2011/093185; WO 2011/093187; WO 2011/093186; DE 102010000168; CN 102115757; CN 102093984; CN 102080069; JP 2011103864; WO 2011/042143; WO 2011/038313; JP 2011055721; WO 2011/025203; JP 2011024534; WO 2011/008231; WO 2011/008232; WO 2011/005786; IN 2009DE01216; DE 102009025996; WO 2010/134096; JP 2010233523; JP 2010220609; WO 2010/095721; WO 2010/064764; US 20100136595; JP 2010051174; WO 2010/024437; WO 2010/011882; WO 2009/108388; US 20090209010; US 20090124515; WO 2009/041470; KR 2009028942; WO 2009/039487; WO 2009/020231; JP 2009005687; CN 101333520; CN 101333521; US 20080248545; JP 2008237110; CN 101275141; WO 2008/118545; WO 2008/115844; CN 101255408; CN 101250506; CN 101250505; WO 2008/098198; WO 2008/096695; WO 2008/071673; WO 2008/073498; WO 2008/065370; WO 2008/067070; JP 2008127301; JP 2008054644; KR 794395; EP 1881066; WO 2007/147827; CN 101078014; JP 2007300852; WO 2007/048235; WO 2007/044688; WO 2007/032540; CN 1900286; CN 1900285; JP 2006340611; WO 2006/126723; KR 2006029792; KR 2006029795; WO 2006/105082; WO 2006/076094; US 2006/0156430; WO 2006/065126; JP 2006129836; CN 1746293; WO 2006/029398; JP 2006034215; JP 2006034214; WO 2006/009334; WO 2005/111216; WO 2005/080572; US 2005/0150002; WO 2005/061699; WO 2005/052152; WO 2005/038033; WO 2005/038018; WO 2005/030944; JP 2005065618; WO 2005/017106; WO 2005/017105; US 20050037411; WO 2005/010166; JP 2005021106; JP 2005021104; JP 2005021105; WO 2004/113527; CN 1472323; JP 2004261121; WO 2004/013339; WO 2004/011648; DE 10234126; WO 2004/003190; WO 2003/087381; WO 2003/078577; US 20030170627; US 20030166176; US 20030150025; WO 2003/057830; WO 2003/052050; CN 1358756; US 20030092658; US 20030078404; US 20030066103; WO 2003/014341; US 20030022334; WO 2003/008563; EP 1270722; US 20020187538; WO 2002/092801; WO 2002/088341; US 20020160950; WO 2002/083868; US 20020142379; WO 2002/072758; WO 2002/064765; US 20020076777; US 20020076774; US 20020076774; WO 2002/046386; WO 2002/044213; US 20020061566; CN 1315335; WO 2002/034922; WO 2002/033057; WO 2002/029018; WO 2002/018558; JP 2002058490; US 20020022254; WO 2002/008269; WO 2001/098461; WO 2001/081585; WO 2001/051622; WO 2001/034780; CN 1271005; WO 2001/011071; WO 2001/007630; WO 2001/007574; WO 2000/078973; U.S. Pat. No. 6,130,077; JP 2000152788; WO 2000/031273; WO 2000/020566; WO 2000/000585; DE 19826821; JP 11235174; U.S. Pat. No. 5,939,318; WO 99/19493; WO 99/18224; U.S. Pat. No. 5,886,157; WO 99/08812; U.S. Pat. No. 5,869,283; JP 10262665; WO 98/40470; EP 776974; DE 19507546; GB 2294692; U.S. Pat. No. 5,516,674; JP 07147975; WO 94/29434; JP 06205685; JP 05292959; JP 04144680; DD 298820; EP 477961; SU 1693043; JP 01047375; EP 281245; JP 62104583; JP 63044888; JP 62236485; JP 62104582; and JP 62019084.

TABLE C
Additional cytochrome P450 enzymes of the present invention.

| Species | Cyp No. | Accession No. | SEQ ID NO |
|---|---|---|---|
| Bacillus megaterium | 102A1 | AAA87602 | 1 |
| Bacillus megaterium | 102A1 | ADA57069 | 2 |
| Bacillus megaterium | 102A1 | ADA57068 | 3 |
| Bacillus megaterium | 102A1 | ADA57062 | 4 |
| Bacillus megaterium | 102A1 | ADA57061 | 5 |
| Bacillus megaterium | 102A1 | ADA57059 | 6 |
| Bacillus megaterium | 102A1 | ADA57058 | 7 |
| Bacillus megaterium | 102A1 | ADA57055 | 8 |
| Bacillus megaterium | 102A1 | ACZ37122 | 9 |
| Bacillus megaterium | 102A1 | ADA57057 | 10 |
| Bacillus megaterium | 102A1 | ADA57056 | 11 |
| Mycobacterium sp. HXN-1500 | 153A6 | CAH04396 | 12 |
| Tetrahymena thermophile | 5013C2 | ABY59989 | 13 |
| Nonomuraea dietziae | | AGE14547.1 | 14 |
| Homo sapiens | 2R1 | NP_078790 | 15 |
| Macca mulatta | 2R1 | NP_001180887.1 | 16 |
| Canis familiaris | 2R1 | XP_854533 | 17 |
| Mus musculus | 2R1 | AAI08963 | 18 |
| Bacillus halodurans C-125 | 152A6 | NP_242623 | 19 |
| Streptomyces parvus | aryC | AFM80022 | 20 |
| Pseudomonas putida | 101A1 | P00183 | 21 |
| Homo sapiens | 2D7 | AAO49806 | 22 |
| Rattus norvegicus | C27 | AAB02287 | 23 |
| Oryctolagus cuniculus | 2B4 | AAA65840 | 24 |
| Bacillus subtilis | 102A2 | O08394 | 25 |
| Bacillus subtilis | 102A3 | O08336 | 26 |
| B. megaterium DSM 32 | 102A1 | P14779 | 27 |
| B. cereus ATCC14579 | 102A5 | AAP10153 | 28 |
| B. licheniformis ATTC1458 | 102A7 | YP 079990 | 29 |
| B. thuringiensis serovar konkukian | X | YP 037304 | 30 |
| str.97-27 | | | |
| R. metallidurans CH34 | 102E1 | YP 585608 | 31 |
| A. fumigatus Af293 | 505X | EAL92660 | 32 |
| A. nidulans FGSC A4 | 505A8 | EAA58234 | 33 |
| A. oryzae ATCC42149 | 505A3 | Q2U4F1 | 34 |
| A. oryzae ATCC42149 | X | Q2UNA2 | 35 |
| F. oxysporum | 505A1 | Q9Y8G7 | 36 |
| G. moniliformis | X | AAG27132 | 37 |
| G. zeae PH1 | 505A7 | EAA67736 | 38 |
| G. zeae PH1 | 505C2 | EAA77183 | 39 |
| M. grisea 70-15 syn | 505A5 | XP 365223 | 40 |
| N. crassa OR74 A | 505A2 | XP 961848 | 41 |
| Oryza sativa* | 97A | | |
| Oryza sativa* | 97B | | |
| Oryza sativa | 97C | ABB47954 | 42 |

The start methionine (“M”) may be present or absent from these sequences.
*See, M.Z. Lv et al., Plant Cell Physiol., 53(6):987-1002 (2012).

In certain embodiments, the present invention provides amino acid substitutions that efficiently remove monooxygenation chemistry from cytochrome P450 enzymes. This system permits selective enzyme-driven aziridination chemistry without competing side reactions mediated by native P450 catalysis. The invention also provides P450-mediated catalysis that is competent for aziridination chemistry but not able to carry out traditional P450-mediated monooxygenation reactions as ‘orthogonal’ P450 catalysis and respective enzyme variants as ‘orthogonal’ P450s. In some instances, orthogonal P450 variants comprise a single amino acid mutation at the axial position of the heme coordination site (e.g., a C400S mutation in the P450 BM3 enzyme) that alters the proximal heme coordination environment. Accordingly, the present invention also provides P450 variants that contain an axial heme mutation in combination with one or more additional mutations described herein to provide orthogonal P450 variants that show enriched diastereoselective and/or enantioselective product distributions. The present invention further provides a compatible reducing agent for orthogonal P450 aziridination catalysis that includes, but is not limited to, NAD(P)H or sodium dithionite.

In certain instances, the cytochrome P450 BM3 enzyme comprises or consists of the amino acid sequence set forth in SEQ ID NO:1. In certain other instances, the cytochrome P450 BM3 enzyme is a natural variant thereof as described, e.g., in J. Y. Kang et al., AMB Express 1:1 (2011), wherein the natural variants are divergent in amino acid sequence from the wild-type cytochrome P450 BM3 enzyme sequence (SEQ ID NO:1) by up to about 5% (e.g., SEQ ID NOS:2-11).

In particular embodiments, the P450 BM3 enzyme variant comprises or consists of the heme domain of the wild-type P450 BM3 enzyme sequence (e.g., amino acids 1-463 of SEQ ID NO: 1) and optionally at least one mutation as described herein. In other embodiments, the P450 BM3 enzyme variant comprises or consists of a fragment of the heme domain of the wild-type P450 BM3 enzyme sequence (SEQ ID NO: 1), wherein the fragment is capable of carrying out the aziridination reactions of the present invention. In some instances, the fragment includes the heme axial ligand and at least one, two, three, four, or five of the active site residues.

In other embodiments, the P450 BM3 enzyme variant comprises at least one or more (e.g., at least two, three, four, five, six, seven, eight, nine, ten, eleven, twelve, thirteen, or all fourteen) of the following amino acid substitutions in SEQ ID NO:1: V78A, F87V, P142S, T175I, A184V, S226R, H236Q, E252G, T268A, A290V, L353V, I366V, T438S, and E442K (SEQ ID NO: 55). In certain instances, the P450 BM3 enzyme variant comprises a T268A mutation alone or in combination with one or more additional mutations such as a C400X mutation (e.g., C400S) in SEQ ID NO:1 (SEQ ID NO: 56). In other instances, the P450 BM3 enzyme variant comprises all fourteen of these amino acid substitutions (i.e., V78A, F87V, P142S, T175I, A184V, S226R, H236Q, E252G, T268A, A290V, L353V, I366V, T438S, and E442K; “BM3-CIS T438S”) in combination with a C400X mutation (e.g., C400S) in SEQ ID NO:1 (SEQ ID NO: 57). In some instances, the P450 BM3 enzyme variant comprises or consists of the heme domain of the BM3-CIS T438S enzyme sequence (e.g., amino acids 1-463 of SEQ ID NO: 1 comprising all fourteen of these amino acid substitutions (SEQ ID NO: 55)).

In some embodiments, the P450 BM3 enzyme variant comprises the axial ligand mutation C400S and substitutions to SEQ ID NO:1: V78A, F87V, P142S, T175I, A184V, S226R, H236Q, E252G, I263F, T268A, A290V, A328V, L353V, I366V, L437V, T438S, E442K (SEQ ID NO: 51). In another embodiment, the heme variant comprises the axial ligand mutation C400S and the following amino acid substitutions: L75A, V78A, F87V, P142S, T175I, L181A, A184V, S226R, H236Q, E252G, I263F, T268A, A290V, L353V, I366V, E442K (SEQ ID NO: 53). In some embodiments, the heme enzyme variant is the P-I263F variant (see, Table D). In some embodiments, the heme enzyme variant is the P411_{BM3} H2-A-10 I263F (see, Table D).

Table D below provides non-limiting examples of cytochrome P450 BM3 variants of the present invention. Each P450 BM3 variant comprises the mutations relative to the wild-type P450 BM3 enzymes as shown.

TABLE D
Mutations present in P450 BM3 variants used in the disclosure.

| P450_{BM3} variant | Mutations relative to wild-type P450_{BM3} (SEQ ID NO: 1) |
|---|---|
| P450_{BM3} | none |
| P450_{BM3}-T268A | T268A |
| P411_{BM3} | C400S |
| P411_{BM3}-T268A | C400S, T268A |
| P450_{BM3}-CIS | V78A, F87V, P142S, T175I, A184V, S226R, H236Q, |
| | E252G, T268A, A290V, L353V, I366V, E442K |
| P450_{BM3}-CIS-T438S | CIS T438S |
| P450_{BM3}-CIS-T438S C400H | P450_{BM3}-CIS T438S, C400H |
| P450_{BM3}-CIS-T438S C400D | P450_{BM3}-CIS T438S, C400D |
| P450_{BM3}-CIS-T438S C400M | P450_{BM3}-CIS T438S, C400M |
| P411_{BM3}-CIS | P450_{BM3}-CIS C400S |
| P411_{BM3}-CIS-T4385 | P450_{BM3}-CIS C400S, T438S |
| P411_{BM3}-CIS-T4385 I263F | P450_{BM3}-CIS C400S, T438S, I263F |
| (P-I263F) | |
| P-I263F-A328V | P450_{BM3}-CIS C400S, T438S, I263F, A328V |
| P-I263F-A328V-L437V | P450_{BM3}-CIS C400S, T438S, I263F, A328V, L437V |
| P411_{BM3}-CIS T438S I263F V87F | P450_{BM3}-CIS C400S, T438S I263F, V87F |
| P411_{BM3}-CIS T438S I263F A268T | P450_{BM3}-CIS C400S, T438S I263F, A268T |
| P411_{BM3}-CIS A268T T438S | P450_{BM3}-CIS C400S, A268T, T438S |
| P411_{BM3} H2-A-10 | P450_{BM3}-CIS C400S, L75A, L181A |
| P411_{BM3} H2-A-10 I263F | P450_{BM3}-CIS C400S, L75A, L181A, I263F |
| P411_{BM3} H2-5-F10 | P450_{BM3}-CIS C400S, L75A, I263A, L437A |
| P411_{BM3} H2-4-D4 | P450_{BM3}-CIS C400S, L75A, M177A, L181A, L437A |

One skilled in the art will understand that any of the mutations listed in Table D can be introduced into any cytochrome P450 enzyme of interest by locating the segment of the DNA sequence in the corresponding cytochrome P450 gene which encodes the conserved amino acid residue as described above for identifying the conserved cysteine residue in a cytochrome P450 enzyme of interest that serves as the heme axial ligand. In certain instances, this DNA segment is identified through detailed mutagenesis studies in a conserved region of the protein (see, e.g., Shimizu et al., Biochemistry 27, 4138-4141, 1988). In other instances, the conserved amino acid residue is identified through crystallographic study (see, e.g., Poulos et al., J. Mol. Biol 195:687-700, 1987). In yet other instances, protein sequence alignment algorithms can be used to identify the conserved amino acid residue. For example, BLAST alignment with the P450 BM3 amino acid sequence as the query sequence can be used to identify the heme axial ligand site and/or the equivalent T268 residue in other cytochrome P450 enzymes.

In other aspects, the disclosure provides chimeric heme enzymes such as, e.g., chimeric P450 polypeptides comprised of recombined sequences from P450 BM3 and at least two, or more distantly related P450 enzymes from Bacillus subtillis or variants. As a non-limiting example, site-directed recombination of three bacterial cytochrome P450s can be performed with sequence crossover sites selected to minimize the number of disrupted contacts within the protein structure. In some embodiments, seven crossover sites can be chosen, resulting in eight sequence blocks. One skilled in the art will understand that the number of crossover sites can be chosen to produce the desired number of sequence blocks, e.g., 1, 2, 3, 4, 5, 6, 7, 8, or 9 crossover sites for 2, 3, 4, 5, 6, 7, 8, 9, or 10 sequence blocks, respectively. In other embodiments, the numbering used for the chimeric P450 refers to the identity of the parent sequence at each block. For example, “12312312” refers to a sequence containing block 1 from P450 #1, block 2 from P450 #2, block 3 from P450 #3, block 4 from P450 #1, block 5 from P450 #2, and so on. A chimeric library useful for generating the chimeric heme enzymes of the invention can be constructed as described in U.S. Pat. Publ. No. US-2012-0171693-A1 to Arnold et al., the disclosure of which is incorporated herein for all purposes.

As a non-limiting example, chimeric P450 proteins comprising recombined sequences or blocks of amino acids from CYP102A1 (Accession No. J04832), CYP102A2 (Accession No. CAB12544), and CYP102A3 (Accession No. U93874) can be constructed. In certain instances, the CYP102A1 parent sequence is assigned “1”, the CYP102A2 parent sequence is assigned “2”, and the CYP102A3 is parent sequence assigned “3”. In some instances, each parent sequence is divided into eight sequence blocks containing the following amino acids (aa): block 1: aa 1-64; block 2: aa 65-122; block 3: aa 123-166; block 4: aa 167-216; block 5: aa 217-268; block 6: aa 269-328; block 7: aa 329-404; and block 8: aa 405-end. Thus, in this example, there are eight blocks of amino acids and three fragments are possible at each block. For instance, “12312312” refers to a chimeric P450 protein of the invention containing block 1 (aa 1-64) from CYP102A1, block 2 (aa 65-122) from CYP102A2, block 3 (aa 123-166) from CYP102A3, block 4 (aa 167-216) from CYP102A1, block 5 (aa 217-268) from CYP102A2, and so on. Non-limiting examples of chimeric P450 proteins include those set forth in Table E (C2G9, X7, X7-12, C2E6, X7-9, C2B12, TSP234). In some embodiments, the chimeric heme enzymes of the invention can comprise at least one or more of the mutations described herein.

| Chimeric | Heme domain | SEQ ID |
|---|---|---|
| P450s | block sequence | NO |
| C2G9 | 22223132 | 43 |
| X7 | 22312333 | 44 |
| X7-12 | 12112333 | 45 |
| C2E6 | 11113311 | 46 |
| X7-9 | 32312333 | 47 |
| C2B12 | 32313233 | 48 |
| TSP234 | 22313333 | 49 |

An enzyme's total turnover number (or TTN) refers to the maximum number of molecules of a substrate that the enzyme can convert before becoming inactivated. In general, the TTN for the heme enzymes of the invention range from about 1 to about 100,000 or higher. For example, the TTN can be from about 1 to about 1,000, or from about 1,000 to about 10,000, or from about 10,000 to about 100,000, or from about 50,000 to about 100,000, or at least about 100,000. In particular embodiments, the TTN can be from about 100 to about 10,000, or from about 10,000 to about 50,000, or from about 5,000 to about 10,000, or from about 1,000 to about 5,000, or from about 100 to about 1,000, or from about 250 to about 1,000, or from about 100 to about 500, or at least about 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, or more. In certain embodiments, the variant or chimeric heme enzymes of the present invention have higher TTNs compared to the wild-type sequences. In some instances, the variant or chimeric heme enzymes have TTNs greater than about 100 (e.g., at least about 100, 150, 200, 250, 300, 325, 350, 400, 450, 500, or more) in carrying out in vitro aziridination reactions. In other instances, the variant or chimeric heme enzymes have TTNs greater than about 1000 (e.g., at least about 1000, 2500, 5000, 10,000, 25,000, 50,000, 75,000, 100,000, or more) in carrying out in vivo whole cell aziridination reactions.

When whole cells expressing a heme enzyme are used to carry out an aziridination reaction, the turnover can be expressed as the amount of substrate that is converted to product by a given amount of cellular material. In general, in vivo aziridination reactions exhibit turnovers from at least about 0.01 to at least about 10 mmol·g_{cdw}⁻¹, wherein g_{cdw} is the mass of cell dry weight in grams. For example, the turnover can be from about 0.1 to about 10 mmol·g_{cdw}⁻¹, or from about 1 to about 10 mmol·g_{cdw}⁻¹, or from about 5 to about 10 mmol·g_{cdw}⁻¹, or from about 0.01 to about 1 mmol·g_{cdw}⁻¹, or from about 0.01 to about 0.1 mmol·g_{cdw}⁻¹, or from about 0.1 to about 1 mmol·g_{cdw}⁻¹, or greater than 1 mmol·g_{cdw}⁻¹. The turnover can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or about 10 mmol·g_{cdw}⁻¹.

When whole cells expressing a heme enzyme are used to carry out a aziridination reaction, the activity can further be expressed as a specific productivity, e.g., concentration of product formed by a given concentration of cellular material per unit time, e.g., in g/L of product per g/L of cellular material per hour (g g_{cdw}⁻¹ h⁻¹). In general, in vivo aziridination reactions exhibit specific productivities from at least about 0.01 to at least about 0.5 g·g_{cdw}⁻¹ h⁻¹, wherein g_{cdw} is the mass of cell dry weight in grams. For example, the specific productivity can be from about 0.01 to about 0.1 g g_{cdw}⁻¹ h⁻¹, or from about 0.1 to about 0.5 g g_{cdw}⁻¹ h⁻¹, or greater than 0.5 g g_{cdw}⁻¹ h⁻¹. The specific productivity can be about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, or about 0.5 g g_{cdw}⁻¹ h⁻¹.

In certain embodiments, mutations can be introduced into the target gene using standard cloning techniques (e.g., site-directed mutagenesis) or by gene synthesis to produce the heme enzymes (e.g., cytochrome P450 variants) of the present invention. The mutated gene can be expressed in a host cell (e.g., bacterial cell) using an expression vector under the control of an inducible promoter or by means of chromosomal integration under the control of a constitutive promoter. Aziridination activity can be screened in vivo or in vitro by following product formation by GC or HPLC as described herein.

The expression vector comprising a nucleic acid sequence that encodes a heme enzyme of the invention can be a viral vector, a plasmid, a phage, a phagemid, a cosmid, a fosmid, a bacteriophage (e.g., a bacteriophage P1-derived vector (PAC)), a baculovirus vector, a yeast plasmid, or an artificial chromosome (e.g., bacterial artificial chromosome (BAC), a yeast artificial chromosome (YAC), a mammalian artificial chromosome (MAC), and human artificial chromosome (HAC)). Expression vectors can include chromosomal, non-chromosomal, and synthetic DNA sequences. Equivalent expression vectors to those described herein are known in the art and will be apparent to the ordinarily skilled artisan.

The expression vector can include a nucleic acid sequence encoding a heme enzyme that is operably linked to a promoter, wherein the promoter comprises a viral, bacterial, archaeal, fungal, insect, or mammalian promoter. In certain embodiments, the promoter is a constitutive promoter. In some embodiments, the promoter is an inducible promoter. In other embodiments, the promoter is a tissue-specific promoter or an environmentally regulated or a developmentally regulated promoter.

It is understood that affinity tags may be added to the N- and/or C-terminus of a heme enzyme expressed using an expression vector to facilitate protein purification. Non-limiting examples of affinity tags include metal binding tags such as His6-tags and other tags such as glutathione S-transferase (GST).

Non-limiting expression vectors for use in bacterial host cells include pCWori, pET vectors such as pET22 or pET22b(+) (EMD Millipore), pBR322 (ATCC37017), pQE™ vectors (Qiagen), pBluescript™ vectors (Stratagene), pNH vectors, lambda-ZAP vectors (Stratagene); ptrc99a, pKK223-3, pDR540, pRIT2T (Pharmacia), pRSET, pCR-TOPO vectors, pET vectors, pSyn_1 vectors, pChlamy_1 vectors (Life Technologies, Carlsbad, Calif.), pGEM1 (Promega, Madison, Wis.), and pMAL (New England Biolabs, Ipswich, Mass.). Non-limiting examples of expression vectors for use in eukaryotic host cells include pXT1, pSG5 (Stratagene), pSVK3, pBPV, pMSG, pSVLSV40 (Pharmacia), pcDNA3.3, pcDNA4/TO, pcDNA6/TR, pLenti6/TR, pMT vectors (Life Technologies), pKLAC1 vectors, pKLAC2 vectors (New England Biolabs), pQE™ vectors (Qiagen), BacPak baculoviral vectors, pAdeno-X™ adenoviral vectors (Clontech), and pBABE retroviral vectors. Any other vector may be used as long as it is replicable and viable in the host cell.

The host cell can be a bacterial cell, an archaeal cell, a fungal cell, a yeast cell, an insect cell, or a mammalian cell.

Suitable bacterial host cells include, but are not limited to, BL21 E. coli, DE3 strain E. coli, E. coli M15, DH5α, DH10β, HB101, T7 Express Competent E. coli (NEB), B. subtilis cells, Pseudomonas fluorescens cells, and cyanobacterial cells such as Chlamydomonas reinhardtii cells and Synechococcus elongates cells. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof. Fungal host cells include, but are not limited to, yeast cells from the genera Saccharomyces (e.g., S. cerevisiae), Pichia (P. Pastoris), Kluyveromyces (e.g., K. lactis), Hansenula and Yarrowia, and filamentous fungal cells from the genera Aspergillus, Trichoderma, and Myceliophthora. Suitable insect host cells include, but are not limited to, Sf9 cells from Spodoptera frugiperda, Sf21 cells from Spodoptera frugiperda, Hi-Five cells, BTI-TN-5B1-4 Trichophusia ni cells, and Schneider 2 (S2) cells and Schneider 3 (S3) cells from Drosophila melanogaster. Non-limiting examples of mammalian host cells include HEK293 cells, HeLa cells, CHO cells, COS cells, Jurkat cells, NS0 hybridoma cells, baby hamster kidney (BHK) cells, MDCK cells, NIH-3T3 fibroblast cells, and any other immortalized cell line derived from a mammalian cell.

In certain embodiments, the present invention provides heme enzymes such as the P450 variants described herein that are active aziridination catalysts inside living cells. As a non-limiting example, bacterial cells (e.g., E. coli) can be used as whole cell catalysts for the in vivo aziridination reactions of the present invention. In some embodiments, whole cell catalysts containing a P450 enzymes variant described herein significantly enhance the total turnover number (TTN) compared to in vitro reactions using isolated P450 enzymes.

B. Compounds

The methods of the invention can be used to provide a number of aziridination products. The aziridination products described herein can be useful starting materials or intermediates for the synthesis of compounds.

The olefinic substrates useful in the present invention are represented by a structure of Formula I:

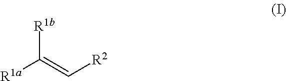

For compounds of Formula I, R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl; Y¹ is C₁₋₈alkylene; each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen. In some embodiments, each aryl contains between 6-14 carbon atoms, each heteroaryl group has from 5 to 8 ring atoms and from 1-3 heteroatoms selected from N, O and S, and each heterocyclyl group has from 1-3 heteroatoms selected from N, O and S.

In certain instances, R^{1a}, R^{1b}, and R² are independently selected from the group consisting of H, C₁₋₁₈alkyl, aryl, heteroaryl, C₁₋₁₂cycloalkyl, and C₃₋₁₀heterocyclyl, each R^{1a}, R^{1b}, and R² is optionally substituted with from 1 to 5 substituents independently selected from the group consisting of C₁₋₃alkyl, alkoxy, and halogen.

In some embodiments, R^{1a} is a substituted phenyl group or a naphthalenyl, wherein the phenyl group is substituted with 1 to 2 a methyl, chloro, or C₁alkyoxy groups.

In some embodiments, R^{1b} is H or methyl.

In some embodiments, R² is H or methyl. In some embodiments, R² is H.

The nitrene precursors useful in the present invention have a structure according to the Formula IIa or IIb:

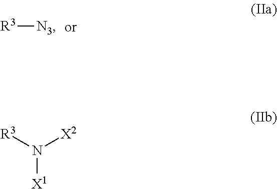

wherein:
R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c}; X¹ is independently selected from the group consisting of H and sodium, and X² is independently selected from the group consisting of halogen, —SO₂R^{a}, —CO₂R^{b}, —PO₃R^{b}R^{c}, optionally X¹ and X² can be taken together to form iodinane; R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl; R^{b} and R^{c} are independently selected from the group consisting of C₁₋₈alkyl, C₃₋₁₂cycloalkyl, aryl, heteroaryl, and C₃₋₈heterocyclyl; wherein within each R³, R^{a}, R^{b}, and R^{c} can be optionally substituted with from 1-5 R^{d} substituents; and each R^{d} is independently selected from the group consisting of C₁₋₃alkyl, halogen, and hydroxy. In some embodiments, each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

In certain instances, R³ is selected from the group consisting of aryl, —SO₂R^{a}, —COR^{a}, —CO₂R^{b}, and —PO₃R^{b}R^{c}; X¹ is independently selected from the group consisting of H and sodium, and X² is independently selected from the group consisting of halogen, —SO₂R^{a}, optionally X¹ and X² can be taken together to form iodinane; R^{a} is independently selected from the group consisting of C₁₋₈alkyl, C₁₋₈alkoxy, and aryl; R^{b} and R^{c} are independently selected from the group consisting of C₁₋₈alkyl, and aryl; wherein within each R³, R^{a}, R^{b}, and R^{c} can be optionally substituted with from 1-5 R^{d} substituents; and each R^{d} is independently selected from the group consisting of C₁₋₃alkyl, and halogen. In some embodiments, each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

In some embodiments, the nitrene precursor has a structure selected from the group consisting of:

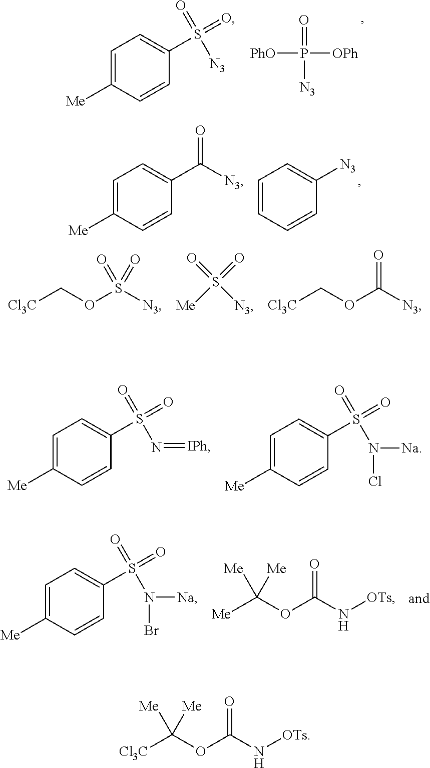

In some instances, the nitrene precursor is

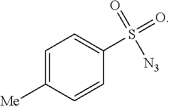 [SMILES: Cc1ccc(S(=O)(=O)N=[N+]=[N-])cc1]

In some embodiments, the aziridination product is a compound according to Formula III:

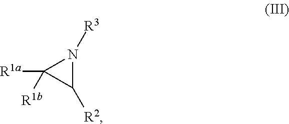

wherein R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl; Y¹ is C₁₋₈alkylene; each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen; R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c}; R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl; R^{b} and R^{c} are independently selected from the group consisting of C₁₋₈alkyl, C₃₋₁₂cycloalkyl, aryl, heteroaryl, and C₃₋₈heterocyclyl, wherein within each R³, R^{a}, R^{b}, and R^{c} can be optionally substituted with from 1-5 R^{d} substituents; and each R^{d} is independently selected from the group consisting of C₁₋₃alkyl, halogen, and hydroxy. In some embodiments, each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

In some instances, R^{1a} and R^{1b} are independently selected from the group consisting of H, C₁₋₈alkyl, aryl, heteroaryl, C₁₋₁₂cycloalkyl, and C₃₋₁₀heterocyclyl; R² is selected from the group consisting of H and C₁₋₈ alkyl; each R^{1a}, R^{1b}, and R² is optionally substituted with from 1 to 3 substituents independently selected from the group consisting of C₁₋₃alkyl, alkoxy, and halogen; and R³ is selected from the group consisting of —SO₂R^{a}, —COR^{a}, —CO₂R^{b}, and —PO₃R^{b}R^{c}; R^{a} is independently selected from the group consisting of C₃₋₁₂cycloalkyl, aryl, heteroaryl, and C₃₋₈heterocyclyl; R^{b} and R^{c} are independently selected from the group consisting of C₃₋₁₂cycloalkyl, aryl, heteroaryl, and C₃₋₈heterocyclyl, wherein within each R³, R^{a}, R^{b}, and R^{c} can be optionally substituted with from 1-2 R^{d} substituents; and each R^{d} is independently selected from the group consisting of C₁₋₃alkyl, halogen, and hydroxy.

In some embodiments, the aziridination product is a compound according to Formula IIIa:

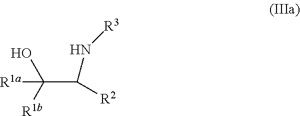

wherein R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl; Y¹ is C₁₋₈alkylene; each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen; R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c}; R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl; R^{b} and R^{c} are independently selected from the group consisting of C₁₋₈alkyl, C₃₋₁₂cycloalkyl, aryl, heteroaryl, and C₃₋₈heterocyclyl, wherein within each R³, R^{a}, R^{b}, and R^{c} can be optionally substituted with from 1-5 R^{d} substituents; and each R^{d} is independently selected from the group consisting of C₁₋₃alkyl, halogen, and hydroxy. In some embodiments, each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

In some instances, R^{1a} and R^{1b} are independently selected from the group consisting of H, C₁₋₈alkyl, aryl, heteroaryl, C₁₋₁₂cycloalkyl, and C₃₋₁₀heterocyclyl; R² is selected from the group consisting of H and C₁₋₈ alkyl; each R^{1a}, R^{1b}, and R² is optionally substituted with from 1 to 3 substituents independently selected from the group consisting of C₁₋₃alkyl, alkoxy, and halogen; and R³ is selected from the group consisting of —SO₂R^{a}, —COR^{a}, —CO₂R^{b}, and —PO₃R^{b}R^{c}; R^{a} is independently selected from the group consisting of C₃₋₁₂cycloalkyl, aryl, heteroaryl, and C₃₋₈heterocyclyl; R^{b} and R^{c} are independently selected from the group consisting of C₃₋₁₂cycloalkyl, aryl, heteroaryl, and C₃₋₈heterocyclyl, wherein within each R³, R^{a}, R^{b}, and R^{c} can be optionally substituted with from 1-2 R^{d} substituents; and each R^{d} is independently selected from the group consisting of C₁₋₃alkyl, halogen, and hydroxy.

In some embodiments, compounds of Formula IIIa are further reaction products of an aziridine ring that has been opened after attack from a nucleophile, such as a hydroxyl group. Compounds of formula IIIa can be produced when the aziridination reactions described herein are performed under aqueous reaction conditions.

In some embodiments, the aziridination product has a structure selected from the group consisting of:

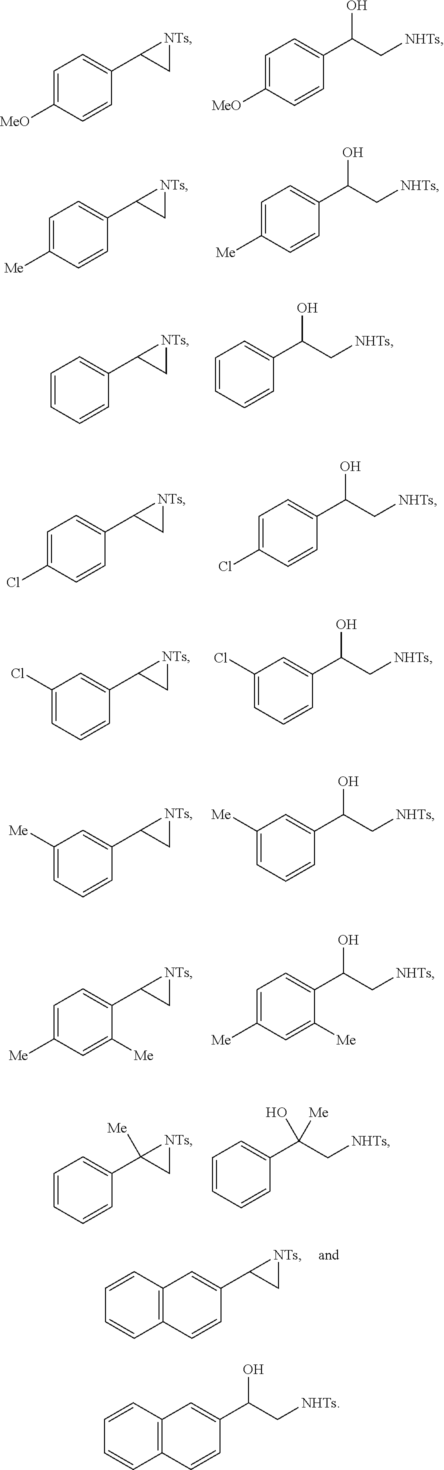

One of skill in the art will appreciate that stereochemical configuration of certain of the products herein will be determined in part by the orientation of the product of the enzymatic step. Certain of the products herein will be “cis” compounds or “Z” compounds. Other products will be “trans” compounds or “E” compounds. One product where cis or trans orientations are possible is the formation of an aziridine ring. The cis configuration of an aziridine ring is when the highest priority substituents are on the same side of the ring (e.g., Formula III when R^{1a} and R² are the highest priority substituents and on the same side of the aziridine ring), while the trans configuration of an aziridine ring is when the highest priority substituents are on the opposite side of the ring.

In certain instances, two cis isomers and two trans isomers can arise from the reaction of an olefin substrate and a nitrene precursor. The two cis isomers are enantiomers with respect to one another, in that the structures are non-superimposable mirror images of each other. Similarly, the two trans isomers are enantiomers. One of skill in the art will appreciate that the absolute stereochemistry of a product—that is, whether a given chiral center exhibits the right-handed “R” configuration or the left-handed “S” configuration—will depend on factors including the structures of the particular substrate and nitrene precursor used in the reaction, as well as the identity of the enzyme. The relative stereochemistry—that is, whether a product exhibits a cis or trans configuration—as well as for the distribution of product mixtures will also depend on such factors.

In certain instances, the product mixtures have cis:trans ratios ranging from about 1:99 to about 99:1. The cis:trans ratio can be, for example, from about 1:99 to about 1:75, or from about 1:75 to about 1:50, or from about 1:50 to about 1:25, or from about 99:1 to about 75:1, or from about 75:1 to about 50:1, or from about 50:1 to about 25:1. The cis:trans ratio can be from about 1:80 to about 1:20, or from about 1:60 to about 1:40, or from about 80:1 to about 20:1 or from about 60:1 to about 40:1. The cis:trans ratio can be about 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, or about 1:95. The cis:trans ratio can be about 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, or about 95:1.

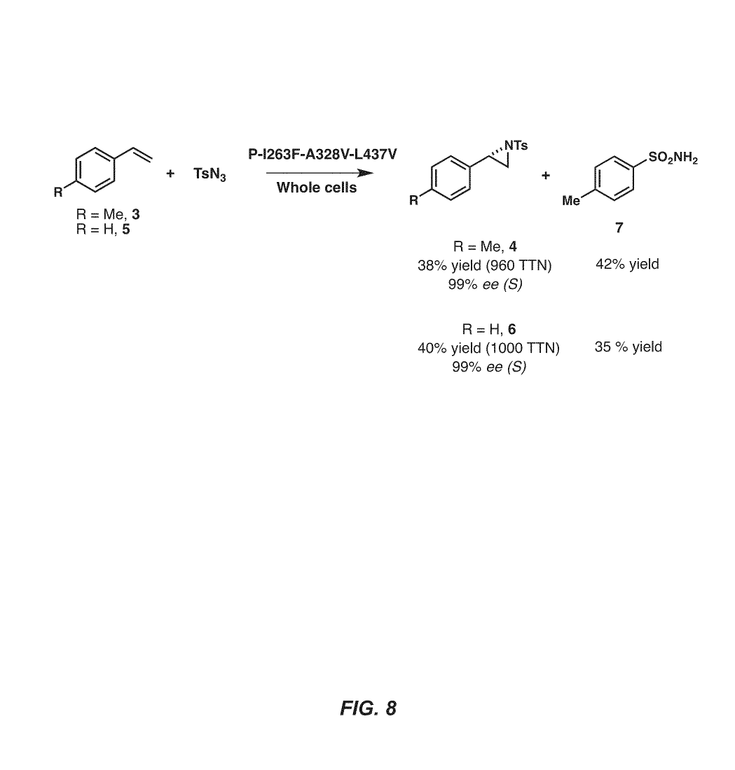

The distribution of product mixture can be assessed in terms of the enantiomeric excess, or “% ee,” of the mixture. The enantiomeric excess refers to the difference in the mole fractions of two enantiomers in a mixture. Taking the reaction scheme in FIG. 8 as a non-limiting example, for instance, the enantiomeric excess of the “S” enantiomer can be calculated using the formula: % ee_{S}=[(χ_{S}−χ_{R})/(χ_{S}+χ_{R})]×100%, wherein χ is the mole fraction for a given enantiomer. The enantiomeric excess of the “R” enantiomer (% ee_{R}) can be calculated in the same manner. In less otherwise specified, % ee is reported as % ee_{S}.

In general, product mixtures exhibit % ee values ranging from about 1% to about 99%, or from about −1% to about −99%. The closer a given % ee value is to 99% (or −99%), the purer the reaction mixture is. The % ee can be, for example, from about −90% to about 90%, or from about −80% to about 80%, or from about −70% to about 70%, or from about −60% to about 60%, or from about −40% to about 40%, or from about −20% to about 20%. The % ee can be from about 1% to about 99%, or from about 20% to about 80%, or from about 40% to about 60%, or from about 1% to about 25%, or from about 25% to about 50%, or from about 50% to about 75%. The % ee can be from about −1% to about −99%, or from about −20% to about −80%, or from about −40% to about −60%, or from about −1% to about −25%, or from about −25% to about −50%, or from about −50% to about −75%. The % ee can be about −99%, −95%, −90%, −85%, −80%, −75%, −70%, −65%, −60%, −55%, −50%, −45%, −40%, −35%, −30%, −25%, −20%, −15%, −10%, 10%, 15%, 20%, 25%, 30%, 35%, 40%, 45%, 50%, 55%, 60%, 65%, 70%, 75%, 80%, 85%, 90%, or about 95%. Any of these values can be % ee_{S} values or % ee_{R} values.

Accordingly, some embodiments of the invention provide methods for producing a plurality of aziridination products having a % ee_{S} of from about −90% to about 90%. In some embodiments, the % ee_{S} is at least 90%. In some embodiments, the % ee_{S} is at least −99%. In some embodiments, the % ee_{R} is from about −90% to about 90%. In some embodiments, the % ee_{R} is at least 90%. In some embodiments, the % ee_{R} is at least −99%.

The methods of the disclosure can also be assessed in terms of the diastereoselectivity and/or enantioselectivity of the aziridination reaction—that is, the extent to which the reaction produces a particular isomer, whether a diastereomer or enantiomer. A perfectly selective reaction produces a single isomer, such that the isomer constitutes 100% of the product. As another non-limiting example, a reaction producing a particular enantiomer constituting 90% of the total product can be said to be 90% enantioselective. A reaction producing a particular diastereomer constituting 30% of the total product, meanwhile, can be said to be 30% diastereoselective. The diastereoselectivity and/or enantioselectivity of an aziridination reaction is dependent on a number of factors including the olefinic substrate, nitrene precursor, and heme enzyme used.

In general, the methods of the invention include reactions that are from about 1% to about 99% diastereoselective. The reactions are from about 1% to about 99% enantioselective. The reaction can be, for example, from about 10% to about 90% diastereoselective, or from about 20%>to about 80%>diastereoselective, or from about 40%>to about 60%) diastereoselective, or from about 1% to about 25% diastereoselective, or from about 25% o to about 50% diastereoselective, or from about 50% to about 75% diastereoselective. The reaction can be about 10%, 15%, 20%, 25%, 30%, 35%, 40%, 45%, 50%, 55%, 60%, 65%, 70%, 75%, 80%, 85%, 90%, or about 95% diastereoselective. The reaction can be from about 10% to about 90% enantioselective, from about 20% to about 80% enantioselective, or from about 40% to about 60% enantioselective, or from about 1% to about 25% enantioselective, or from about 25% to about 50% enantioselective, or from about 50% to about 75% enantioselective. The reaction can be about 10%, 15%, 20%, 25%, 30%, 35%, 40%, 45%, 50%, 55%, 60%, 65%, 70%, 75%, 80%, 85%, 90%, or about 95% enantioselective. Accordingly some embodiments of the disclosure provide methods wherein the reaction is at least 30% to at least 90% diastereoselective. In some embodiments, the reaction is at least 30% to at least 90% enantioselective.

C. Reaction Conditions

The methods of the invention include forming reaction mixtures that contain the heme enzymes described herein. The heme enzymes can be, for example, purified prior to addition to a reaction mixture or secreted by a cell present in the reaction mixture. The reaction mixture can contain a cell lysate including the enzyme, as well as other proteins and other cellular materials. Alternatively, a heme enzyme can catalyze the reaction within a cell expressing the heme enzyme. Any suitable amount of heme enzyme can be used in the methods of the invention. In general, aziridination reaction mixtures contain from about 0.01 mol % to about 10 mol % heme enzyme with respect to the nitrene precursor and/or olefinic substrate. The reaction mixtures can contain, for example, from about 0.01 mol % to about 0.1 mol % heme enzyme, or from about 0.1 mol % to about 1 mol % heme enzyme, or from about 1 mol % to about 10 mol % heme enzyme. The reaction mixtures can contain from about 0.05 mol % to about 5 mol % heme enzyme, or from about 0.05 mol % to about 0.5 mol % heme enzyme. The reaction mixtures can contain about 0.1, 0.2, 0.3, 0.4, 0.5, 0.6, 0.7, 0.8, 0.9, or about 1 mol % heme enzyme.

The concentration of olefinic substrate and nitrene precursor are typically in the range of from about 100 μM to about 1 M. The concentration can be, for example, from about 100 μM to about 1 mM, or about from 1 mM to about 100 mM, or from about 100 mM to about 500 mM, or from about 500 mM to 1 M. The concentration can be from about 500 μM to about 500 mM, 500 μM to about 50 mM, or from about 1 mM to about 50 mM, or from about 15 mM to about 45 mM, or from about 15 mM to about 30 mM. The concentration of olefinic substrate or nitrene precursor can be, for example, about 100, 200, 300, 400, 500, 600, 700, 800, or 900 μM. The concentration of olefinic substrate or nitrene precursor can be about 1, 2.5, 5, 7.5, 10, 15, 20, 25, 30, 35, 40, 45, 50, 55, 60, 65, 70, 75, 80, 85, 90, 95, 100, 150, 200, 250, 300, 350, 400, 450, or 500 mM.

Reaction mixtures can contain additional reagents. As non-limiting examples, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, isopropanol, glycerol, tetrahydrofuran, acetone, acetonitrile, and acetic acid), salts (e.g., NaCl, KCl, CaCl₂, and salts of Mn²⁺ and Mg²⁺), denaturants (e.g., urea and guandinium hydrochloride), detergents (e.g., sodium dodecylsulfate and Triton-X 100), chelators (e.g., ethylene glycol-bis(2-aminoethylether)-N,N,N′,N′-tetraacetic acid (EGTA), 2-({2-[Bis(carboxymethyl)amino]ethyl}(carboxymethyl)amino)acetic acid (EDTA), and 1,2-bis(o-aminophenoxy)ethane-N,N,N′,N′-tetraacetic acid (BAPTA)), sugars (e.g., glucose, sucrose, and the like), and reducing agents (e.g., sodium dithionite, NADPH, dithiothreitol (DTT), 3-mercaptoethanol (BME), and tris(2-carboxyethyl)phosphine (TCEP)). Buffers, cosolvents, salts, denaturants, detergents, chelators, sugars, and reducing agents can be used at any suitable concentration, which can be readily determined by one of skill in the art. In general, buffers, cosolvents, salts, denaturants, detergents, chelators, sugars, and reducing agents, if present, are included in reaction mixtures at concentrations ranging from about 1 μM to about 1 M. For example, a buffer, a cosolvent, a salt, a denaturant, a detergent, a chelator, a sugar, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M. In some embodiments, a reducing agent is used in a sub-stoichiometric amount with respect to the olefin substrate and the nitrene precursor. Cosolvents, in particular, can be included in the reaction mixtures in amounts ranging from about 1% v/v to about 75% v/v, or higher. A cosolvent can be included in the reaction mixture, for example, in an amount of about 5, 10, 20, 30, 40, or 50% (v/v).

Reactions are conducted under conditions sufficient to catalyze the formation of an aziridination product. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 6 to about 10. The reactions can be conducted, for example, at a pH of from about 6.5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Reactions can be conducted under aerobic conditions or anaerobic conditions. Reactions can be conducted under an inert atmosphere, such as a nitrogen atmosphere or argon atmosphere. In some embodiments, a solvent is added to the reaction mixture. In some embodiments, the solvent forms a second phase, and the aziridination reaction occurs in the aqueous phase. In some embodiments, the heme enzymes is located in the aqueous layer whereas the substrates and/or products occur in an organic layer. Other reaction conditions may be employed in the methods of the invention, depending on the identity of a particular heme enzyme, olefinic substrate, or nitrene precursor.

Reactions can be conducted in vivo with intact cells expressing a heme enzyme of the invention. The in vivo reactions can be conducted with any of the host cells used for expression of the heme enzymes, as described herein. A suspension of cells can be formed in a suitable medium supplemented with nutrients (such as mineral micronutrients, glucose and other fuel sources, and the like). Aziridination yields from reactions in vivo can be controlled, in part, by controlling the cell density in the reaction mixtures. Cellular suspensions exhibiting optical densities ranging from about 0.1 to about 50 at 600 nm can be used for aziridination reactions. Other densities can be useful, depending on the cell type, specific heme enzymes, or other factors.

### IV. Examples

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Aziridination Activity of Cytochrome P450 Variants and Other Heme Proteins

This example illustrates the aziridination activity of known cytochrome P450 variants and other heme containing enzymes.

Previous studies have shown that cytochrome P450 and mutants thereof can catalyze a wide variety of chemical reactions including cyclopropanation, sulfinde imidation, and C—H amination. In order to assess the potential of a cytochrome P450 or a mutant thereof to catalyze an aziridination reaction, engineered variants of cytochrome P450_{BM3} and P411_{BM3}-CIS-T438S, previously found to be effective for intramolecular C—H amination and sulfide imidation, were tested for aziridination activity. Cytochrome P450_{BM3} is a naturally occurring enzyme found in the soil bacterium bacillus megaterium, and P411_{BM3}-CIS-T438S is a 14 mutation variant of P450_{BM3} (see Table 2 for mutations from wild-type P450_{BM3}). P411_{BM3}-CIS-T438S is called a “P411” due to the change in the characteristic CO-bound Soret peak from 450 to 411 nm effected by mutation of the cysteine residue that coordinates the heme iron to serine (C400S). This axial cysteine is completely conserved in cytochrome P450s and is required for the native monooxygenase activity. Thus, the P411 enzyme is no longer a “cytochrome P450”, nor does it exhibit its native hydroxylation activity. However, the C400S mutation increases the non-natural carbene transfer activities of P450_{BM3} and other P450s. Two crystal structures of P411 variants of P450_{BM3} show that S400 coordinates the iron and causes no significant structural perturbation in the substrate binding pocket.

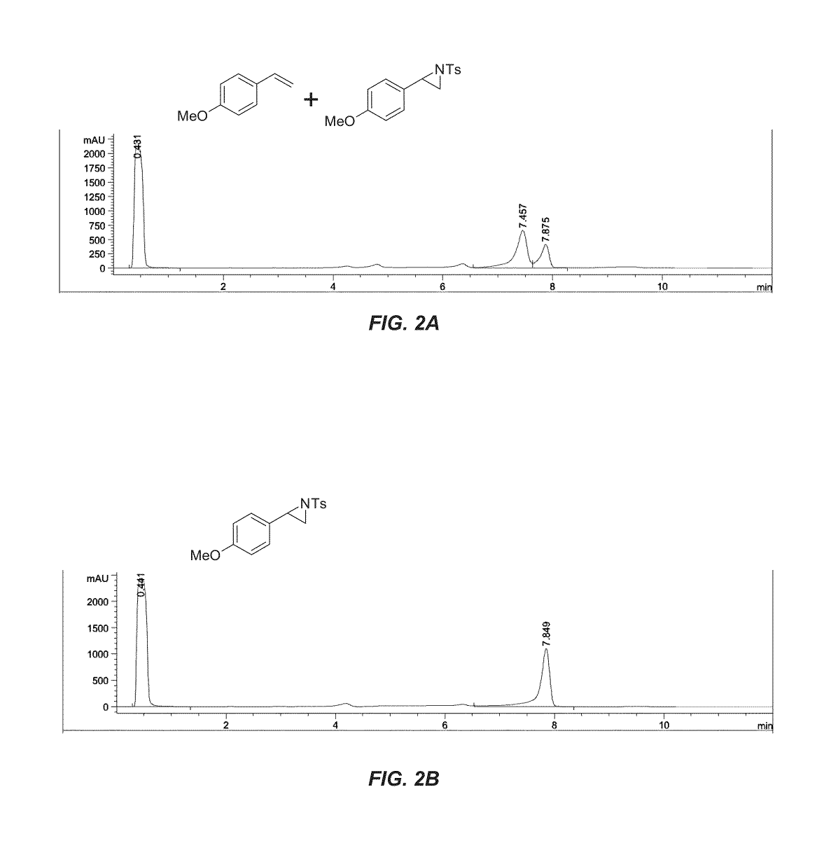

The aziridination activity of P411_{BM3}-CIS-T438S was tested using styrene derivatives as the olefin substrate and tosyl azide (TsN₃) as the nitrene precursor (Table 1). Tosyl azide was completely consumed in this reaction, the major product of which was the azide reduction product p-toluenesulfonamide (>300 total turnovers (TTN), not shown in Table 1). Amidoalcohol 2 appeared as a minor product. Control experiments showed that the desired aziridine product rapidly decomposes under aqueous reaction conditions to the corresponding amidoalcohol 2 (FIGS. 2A-B, 3A-D, and 4A-D). Degradation of this aziridine product has also been observed in studies with small-molecule catalysts (Ando, T.; et al., Tetrahedron 54, 13485-13494 (1998) and Kiyokawa, K. et al., Org. Lett., 15, 4858-4861 (2013)). It was thus inferred that production of 2 is directly related to the nitrene transfer activity of the enzyme toward olefin 1.

TABLE 1
Total turnovers (TTN) to product for aziridination catalyzed by purified holoenzymes
P411_{BM3}-CIS-T438S (P) and P411_{BM3}-CIS-T438S-I263F (P-I263F) with selected styrenyl
olefins 1, 3, and 5 and tosyl azide.^{a}

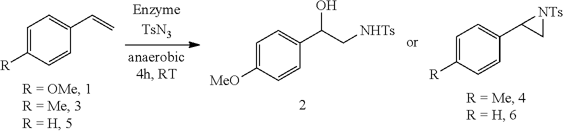

| Enzyme | TTN 2 ^{b} | TTN 4 | TTN 6 |
|---|---|---|---|
| P411_{BM3}-CIS-T438S (P) | 15 | 8 | 5 |
| P-I263F | 150 | 160 | 190 |

^{a}Reactions were performed in 0.1 M KPi buffer pH = 8.0 using 0.2 mol % enzyme and NADPH as reductant, with 2.5 mM tosyl azide and 7.5 mM olefin. Detailed reaction conditions can be found in the supporting information.

^{b} TTN=Total turnover number. TTNs were determined by HPLC analysis.

This low level of nitrene transfer activity to 4-methoxystyrene olefin of the P411_{BM3}-CIS-T438S enzyme prompted investigation of other variants. A small set of cytochrome P450_{BM3} variants and heme proteins prepared for other studies were chosen in order to assess how changes in the protein sequence affect nitrene transfer to olefin substrates. Table 2 shows the variants of the cytochrome P450_{BM3} mutants tested, and Tables 3 and 4 illustrate the results of these tests.

TABLE 2
Mutations present in P450 BM3 variants tested.

| Enzyme | Mutations relative to wild-type P450_{BM3} |
|---|---|
| P450_{BM3} | none |
| P450_{BM3}-CIS | V78A, F87V, P142S, T175I, A184V, S226R, H236Q, |
| | E252G, T268A, A290V, L353V, I366V, E442K |
| P450_{BM3}-CIS T438S | P450_{BM3}-CIS T438S |
| P450_{BM3}-CIS T438S C400H | P450_{BM3}-CIS T438S, C400H |
| P450_{BM3}-CIS T438S C400D | P450_{BM3}-CIS T438S, C400D |
| P450_{BM3}-CIS T438S C400M | P450_{BM3}-CIS T438S, C400M |
| P411_{BM3}-CIS | P450_{BM3}-CIS C400S |
| P411_{BM3}-CIS T438S | P450_{BM3}-CIS T438S, C400S |
| P411_{BM3}-CIS A268T T438S | P450_{BM3}-CIS A268T, C400S, T438S |
| P411_{BM3}-H2-5-F10 | P450_{BM3}-CIS L75A, I263A, C400S, L437A |
| P411_{BM3}-H2-A-10 | P450_{BM3}-CIS L75A, L181A, C400S |
| P411_{BM3}-H2-4-D4 | P450_{BM3}-CIS L75A, M177A, L181A, C400S, L437A |
| P411_{BM3} | P450_{BM3}-C400S |
| P450_{BM3}-T268A | T268A |
| P411_{BM3}-T268A | P450_{BM3}-T268A, C400S |
| P411_{BM3}-CIS T438S I263F | P450_{BM3}-CIS T438S, I263F, C400S |
| (P-I263F) | |
| P411_{BM3}-CIS T438S I263F | P450_{BM3}-CIS T438S, I263F, C400S, V87F |
| V87F | |
| P411_{BM3}-CIS T438S I263F | P450_{BM3}-CIS T438S, I263F, C400S, A268T |
| A268T | |

TABLE 3
Panel of P450_{BM3} purified enzymes tested for aziridination reactivity with 4-methoxystyrene and tosyl azide.^{a}

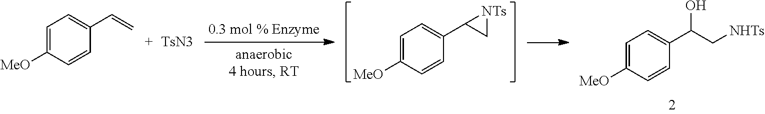

| Entry | Enzyme | TTN 2 |
|---|---|---|
| 1 | P411_{BM3}-CIS T438S (P) | 15 |
| 2 | P450_{BM3}-CIS T438S | <1 |
| 3 | P450_{BM3}-CIS T438S C400H | 3 |
| 4 | P450_{BM3}-CIS T438S C400D | 4 |
| 5 | P450_{BM3}-CIS T438S C400M | 4 |
| 6 | P411_{BM3}-CIS A268T T438S | <1 |
| 7 | P411_{BM3}-H2-5-F10 | 8 |
| 8 | P411_{BM3}-H2-A-10 | 4 |
| 9 | P411_{BM3}-H2-4-D4 | 5 |
| 10 | P450_{BM3} | <1 |
| 11 | P411_{BM3} | 3 |
| 12 | P450_{BM3}-T268A | 2 |
| 13 | P411_{BM3}-T268A | 4 |
| 14 | P411_{BM3}-CIS T438S I263F (P-I263F) | 150 |
| 14 | P411_{BM3}-CIS T438S I263F V87F | 19 |
| 15 | P411_{BM3}-CIS T438S I263F A268T | <1 |

^{a}“P411” denotes Ser-mutated (C400S) variant of cytochrome P450_{BM3}. Variant IDs and specific amino acid substitutions in each can be found in Table 2. TTN—total turnover number.

TABLE 4
Heme and other heme-containing proteins tested for activity in the above reaction
(Table 3) with 4-methoxystyrene. Myoglobin and cytochrome c were purchased as lyophilized
powder from Sigma Aldrich. P450Rhf mutants were expressed and purified as described in the
methods section; P450CYP119 was expressed and purified as described in Heel, T. et al.,
ChemBioChem., 15, 2556(2014).

| Entry | Catalyst | TTN 2 |
|---|---|---|
| 1 | Hemin | <1 |
| 2 | Hemin + BSA | <1 |
| 3 | Myoglobin (horse heart) | <1 |
| 4 | Oytochrome c (bovine heart) | <1 |
| 5 | CYP119 C317S | 7 |
| 6 | CYP119 T213A C317H | <1 |
| 7 | P450_{Rhf} | <1 |

P450_{BM3} sequences lacking the C400S and/or T268A mutations were not active, nor did the Fe(II)-protoporphyrin IX (PPIX) cofactor catalyze aziridination under these conditions. Mutants differing from P411_{BM3}-CIS-T438S by 2-5 alanine mutations in the active site showed some aziridination activity (4-8 TTN), but none was more productive than P411_{BM3}-CIST438S. A set of enzymes containing different axial mutations were tested, including the S400H, S400D, and S400M mutants of P411_{BM3}-CIS-T438S. These enzymes were also only weakly active, giving 2 at levels lower than P411_{BM3}-CIS-T438S (3-4 TTN). Myoglobin (horse heart), cytochrome c (bovine heart), and cytochrome P450_{Rhf} (from Rhodococcus sp. NCIMB 9784) were all inactive for this intermolecular aziridination (Table 4). An engineered variant of the thermostable cytochrome P450 from Sulfolobus acidocaldarius, CYP119, that contained an axial cysteine-to-serine mutation (C317S) did catalyze low levels of aziridination (˜7 TTN). This demonstrates that mutations previously described to activate non-natural nitrene-transfer activity in P450_{BM3} can confer measurable activity on other P450s as well.

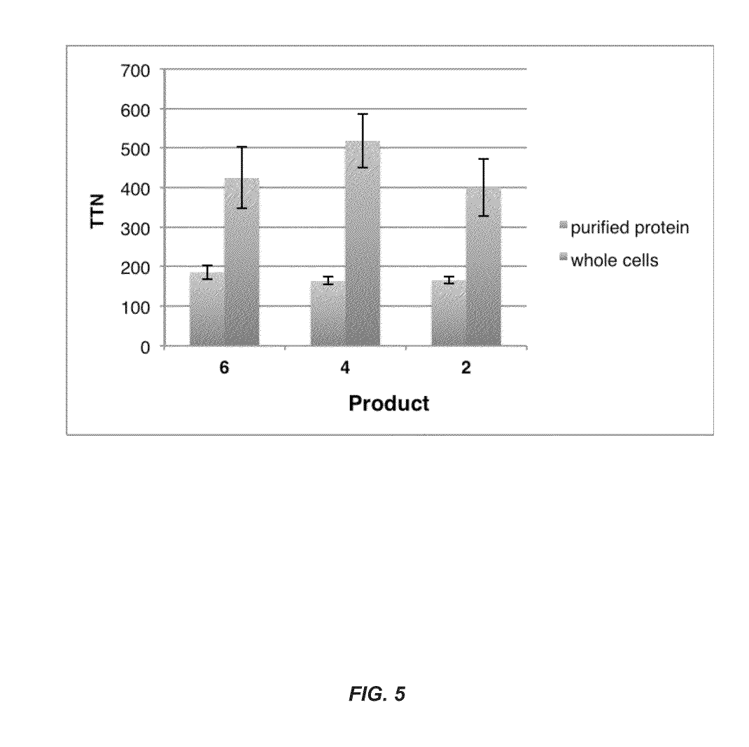

Of all the enzymes tested, a variant of P411BM3-CIS-T438S having a single active-site substitution, I263F, was the most active toward 4-methoxystyrene, providing 150 total turnovers in the formation of amido-alcohol 2 from 4-methoxystyrene (Table 3). P-I263F was even more productive when the reactions were carried out using whole Escherichia coli cells expressing this enzyme (FIG. 5), consistent with our previous observations that enzyme-catalyzed metal-nitrenoid and metal-carbenoid transfer activities improved when the reactions were performed with whole cells. No aziridine product was observed when cells not expressing the P411 catalyst were used.

### Example 2

### Optimizing Cytochrome P450 Aziridination Activity

This example illustrates bacterial cytochrome P450s that are engineered to catalyze highly stereoselective nitrene transfers to olefin substrates to make aziridines.

The P-I263F enzyme identified in the initial studies of enzyme catalyzed aziridination provided enough aziridine product in whole-cell reactions to allow for screening variants in 96-well plate format. Thus, further improvement of aziridination productivity was sought by mutagenesis of this enzyme and screening for aziridination productivity. Site-saturation mutagenesis (SSM) libraries were created at several active site positions that were previously shown to influence productivity and enantioselectivity in other non-natural reactions (A78, L181, T438, A328). Screening of these single SSM libraries for aziridination of 4-methylstyrene (3) identified P-I263F-A328V, with slightly improved yield and substantially improved % ee (96% ee_{S}; entry 4, Table 5). Another round of SSM performed on this variant at additional active site positions (F87, T268, L437) resulted in P-I263F-A328V-L437V with improved aziridine yield and a further increase in enantioselectivity (99% ee_{S}). The P-I263F-L437V and P-I263F-A328V mutants were both less selective than P-I263F-A328V-L437V, demonstrating that both new mutations contribute to the very high enantioselectivity. Importantly, the yield of sulfonamide side product 7 diminished over the course of active site evolution, to the extent that aziridine 4 became the major product of the reaction catalyzed by P-I263F-A328V-L437V.

TABLE 5
Improvement in yield and % ee for aziridine product 4 with active-site evolution of P411_{BM3}CIS-T438S (P).^{a}

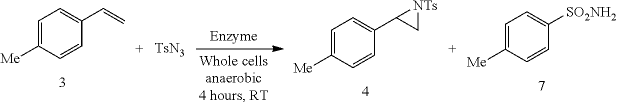

| Entry | Enzyme | % yield 4 | % yield 7 | % ee 4 |
|---|---|---|---|---|
| 1 | No enzyme | 0 | 95 | nd |
| 2 | P411_{BM3}-CIS-T438S | 1.1 | 95 | 25 |
| 3 | P-I263F | 40 | 54 | 55 |
| 4 | P-I263F-A328V | 43 | 50 | 96 |
| 5 | P-I263F-L437V | 37 | 52 | 95 |
| 6 | P-I263F-A328V-L437V | 55 | 43 | 99 |

^{a}Reactions were carried out using whole E. coli cells resuspended in M9-N reaction buffer under anaerobic conditions, with 2.5 mM tosyl azide and 7.5 mM 4-methylstyrene. Yield is based on tosyl azide. See methods for detailed reaction set up and quantification procedures.
^{b}% ee determined by SFC analysis and calculated as (S − R) / (S + R).
^{c}‘No enzyme’ reactions were carried out using whole cells with no P411 enzyme expressed, as described in the SI methods

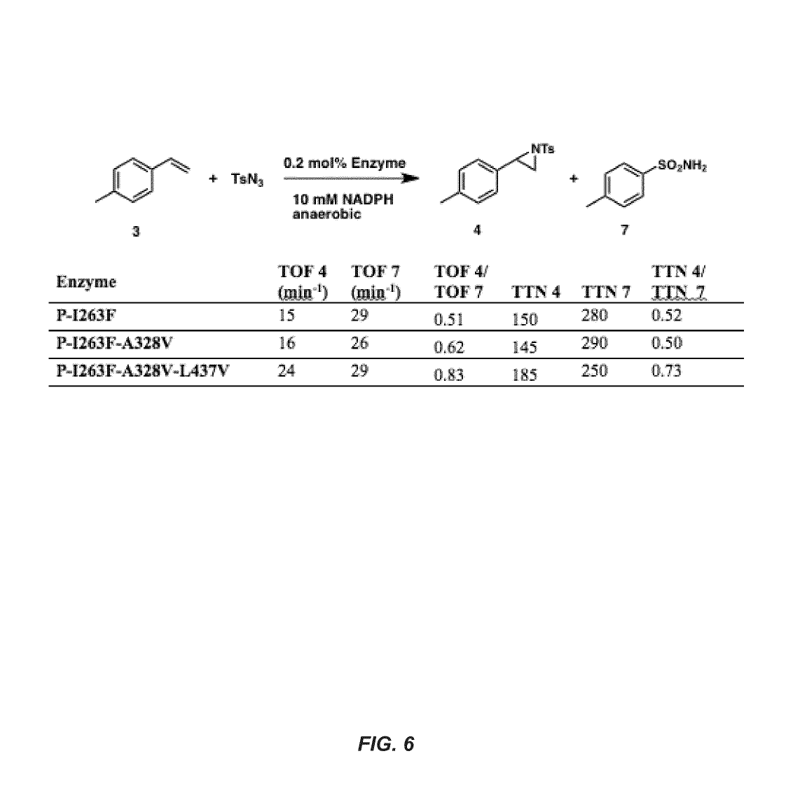

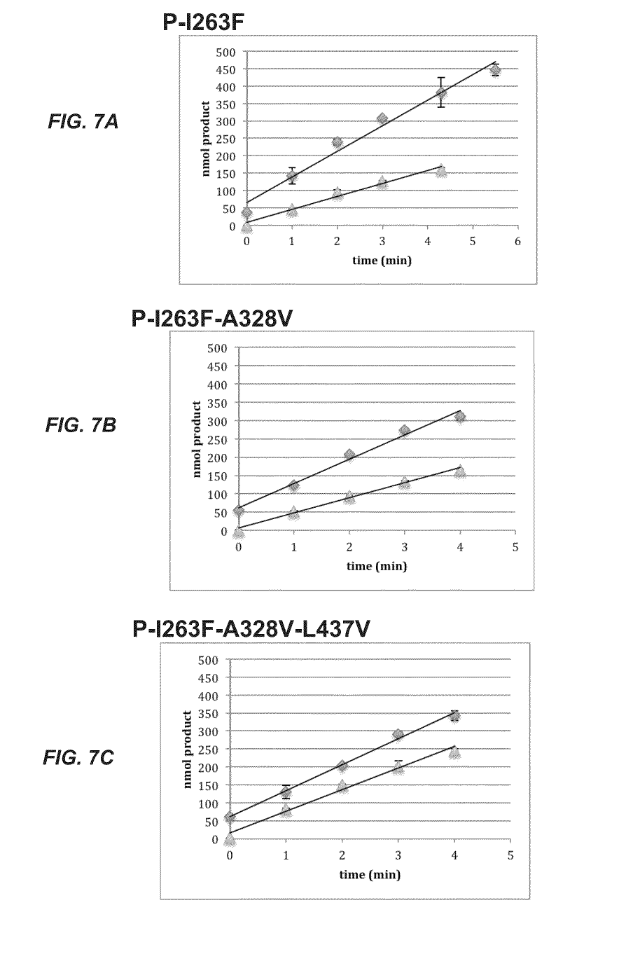

Because the azide is fully consumed in these reactions, the improved aziridine yield could result from either an increase in the rate of aziridine formation or a decrease in the rate of competing azide reduction, or from a combination of both. To address this, initial rates of reaction were measured with the PI263F, P-I263F-A328V, and P-I263F-A328V-L437V enzymes as purified holoenzymes (FIGS. 6 and 7A-C). Initial rates of aziridination for the purified enzymes reflected the yield improvements observed in whole cells: P-I263F and P-I263FA328V have similar turnover frequencies (15-16 min⁻¹), while P-I263F-A328V-L437V, having both new mutations, was improved (TOF ˜24 min⁻¹). The initial turnover frequency of sulfonamide formation in vitro was similar for all the enzymes, and faster than aziridine formation (TOFs ˜26-29 min⁻¹.

### Example 3

### Productivity and Enantioselectivity of Select Cytochrome P450 Enzymes

This example illustrates the aziridination productivity and enantioselectivity of P-I263F-A328V-L437V when reacted with different substrates. This example also illustrates the aziridination productivity and enantioselectivity using enzyme variant P411_{BM3} H2-A-10 I263F.

Having obtained a variant capable of high productivity and enantioselectivity for the aziridination of 4-methylstyrene (3), whole-cell reactions with different substituted styrene substrates were investigated (Table 6). No correlation between the electronics of the aryl substituent and the productivity of the enzyme were observed. In general, the evolved enzyme was more productive with styrenes substituted at the 4-position, though the highest productivity was observed with styrene itself. The evolved enzyme provided 600 catalytic turnovers for the formation of aziridine 6, corresponding to a 70% yield of 6 (entry 3 in Table 6). With higher styrene and tosyl azide loading, the enzyme catalyzed 1,000 turnovers for aziridination, while retaining high (S)-selectivity (99% ee) (FIG. 8). Both 3-methylstyrene and 3-chlorostyrene were significantly less reactive than their 4-substituted counterparts, giving 85 and 21 turnovers, respectively, compared to 450 and 290 turnovers (entries 2, 4, 5, 6 in Table 6). The evolved enzyme is an exceptionally enantioselective aziridination catalyst with styrene entries 2-4 (Table 6), giving 99% ee in favor of the (S)-enantiomer with these three substrates. Both 4-methoxystyrene and α-methylstyrene (entries 1 and 8 in Table 6) gave exclusively racemic amido-alcohol product. Formation of the amido-alcohol product from these substrates may result from carbocation stabilization at the benzylic position due to the resonance and hyperconjugative stabilization provided by the respective p-OMe and α-Me groups, leading to decomposition of the aziridine product and subsequent carbocation quenching with water.

TABLE 6
Substrate aziridination with P-I263F-A328V-L437V showing productivity in terms of
TTN and selectivity in % ee for each product.^{a}

| Entry | Olefin | Product | TTN | % yield | % ee ^{b} |
|---|---|---|---|---|---|
| 1 | 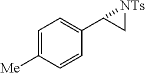 | 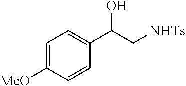 | 390 | 47 | rac |
| 2 | 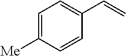 | 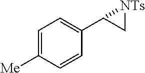 | 450 | 55 | 99 |
| 3 | 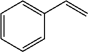 | 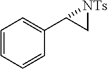 | 600 | 70 | 99 |
| 4 | 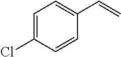 | 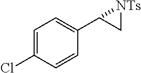 | 290 | 36 | 99 |
| 5 | 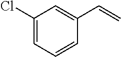 | 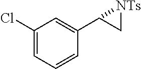 | 21 | 2 | 95 |
| 6 | 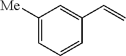 | 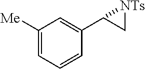 | 85 | 10 | 95 |
| 7 | 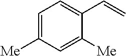 | 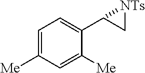 | 130 | 15 | 81 |
| 8 | 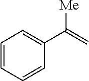 | 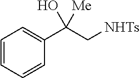 | 83 | 10 | rac |
| 9 | 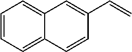 | 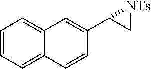 | 53 | 6 | 88 |

^{a}Reactions were carried out with whole cells expressing P-I263F-A328V-L437V under anaerobic conditions, with 2.5 mM tosyl azide and 7.5 mM olefin. Reactions were allowed to proceed for 4 hours at room temperature.
^{b} % ee determined as (S − R) / (S + R). Absolute configurations were assigned based on analogy to 6. rac = racemic.

Although previous work has highlighted the importance of modulating heme electronic properties to access non-natural reactivity (McIntosh, J. A.; et al., Angew. Chem., Int. Ed. 52, 9309-9312 (2013); Hyster, T. K.; et al., J. Am. Chem. Soc., 136, 15505-15508 (2014); Coelho, P. S.; et al., Nat. Chem. Biol. 9, 485-487 (2013)), here it was observed that strong gains in aziridination activity are brought about by mutations on the distal heme side, suggesting that their effect may be the result of improving substrate binding and orientation, a hallmark of enzyme catalysis that is notable for a new-to-nature reaction such as P450-catalyzed nitrene transfer.

P-I263F-A328V-L437V is an exceptionally (S)-selective aziridination catalyst with olefin entries 2-4 (Table 6), giving 99% ee in favor of the (S)-enantiomer with these three substrates. Also identified in this work is the P411_{BM3} H2-A-10 I263F enzyme variant which is an I263F mutant of the P411_{BM3} H2-A-10 enzyme identified in a previous study. The P411_{BM3} H2-A-10 I263F enzyme is able to catalyze the aziridination reaction with enantioselectivity that favors the R-enantiomer (84% ee in favor of (R)-enantiomer, see reaction scheme below).

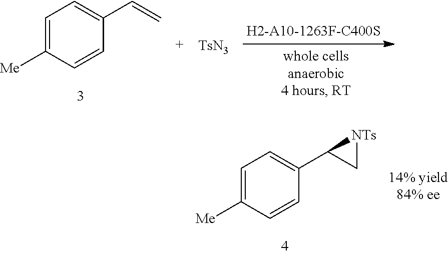

### Example 4

### Synthesis of Substrates and Standards

The following example illustrates the synthesis of substrates and standards.

### N-(2-hydroxy-2-(4-methoxyphenyl)ethyl)-4-methylbenzenesulfonamide (2)

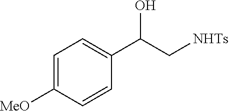

Synthesized as previously reported in Srinivas, B. et al., J. Mol. Catal. A: Chem., 261, 1-5 (2007).

¹H NMR (400 MHz, CDCl₃): δ 7.72 (d, 2H, J=8.1 Hz), 7.29 (d, 2H, J=8.3 Hz), 7.19 (d, 2H, J=8.6 Hz), 6.84 (d, 2H, 8.6 Hz), 5.06 (dd, 1H, J=8.1, 4.6 Hz), 4.73 (dd, 1H, J=8.7, 3.7 Hz), 3.78 (s, 3H), 3.20 (ddd, 1H, J=13.3, 8.1, 3.7 Hz), 3.01 (ddd, 1H, J=13.2, 8.6, 4.6 Hz), 2.42 (s, 3H)

¹³C NMR (101 MHz, CDCl₃): δ 159.66, 143.69, 136.86, 133.00, 129.90, 127.26, 127.21, 114.16, 72.50, 55.44, 50.30, 21.66

HRMS (FAB+): calculated for C₁₆H₁₈NO₄S ([M+H]+): 320.0956. found: 320.0950.

### N-(p-Tolylsulfonyl)-2-(p-methylphenyl)aziridine (4)

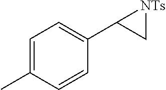 [SMILES: Cc1ccc(C2CN2S(=O)(=O)c2ccc(C)cc2)cc1]

Synthesized as previously reported in Ando, T et al., Tetrahedron, 54, 13485-13494 (1998) with spectral data in agreement with literature reported values (Huang, C. Y. et al., J. Am. Chem. Soc., 134, 9541-9544 (2012); Kiyokawa, K. et al., Org. Lett. 15, 4858-4861 (2013); Evans. D. et al., J. Am. Chem. Soc., 116, 2742-2753 (1994)).

¹H NMR (300 MHz, CDCl₃): δ 7.86 (d, 2H, J=8.3 Hz), 7.32 (d, 2H, J=8.3 Hz), 7.10 (s, 4H), 3.74 (dd, 1H, J=7.2, 4.5 Hz), 2.97 (d, 1H, J=7.2 Hz), 2.43 (s, 3H), 2.38 (d, 1H, J=4.5 Hz), 2.31 (s, 3H).

### N-(p-Tolylsulfonyl)-2-phenylaziridine (6)

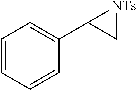 [SMILES: Cc1ccc(S(=O)(=O)N2CC2c2ccccc2)cc1]

Synthesized as previously reported in Ando, T et al., Tetrahedron, 54, 13485-13494 (1998) with spectral data in agreement with literature reported values (Huang, C. Y. et al., J. Am. Chem. Soc., 134, 9541-9544 (2012); Kiyokawa, K. et al., Org. Lett. 15, 4858-4861 (2013); Evans. D. et al., J. Am. Chem. Soc., 116, 2742-2753 (1994)).

¹H NMR (300 MHz, CDCl₃): δ 7.87 (d, 2H, J=8.3 Hz), 7.19-7.36 (m, 7H), 3.77 (dd, 1H, J=7.2, 4.5 Hz), 2.98 (d, 1H, J=7.2 Hz), 2.43 (s, 3H), 2.39 (d, 1H, J=4.5 Hz)

### N-(p-Tolylsulfonyl)-2-(p-methoxyphenyl)aziridine

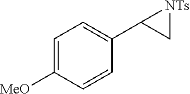 [SMILES: COc1ccc(C2CN2S(=O)(=O)c2ccc(C)cc2)cc1]

Synthesized as previously reported in Ando, T et al., Tetrahedron, 54, 13485-13494 (1998) with spectral data in agreement with literature reported values (Huang, C. Y. et al., J. Am. Chem. Soc., 134, 9541-9544 (2012); Kiyokawa, K. et al., Org. Lett. 15, 4858-4861 (2013); Evans. D. et al., J. Am. Chem. Soc., 116, 2742-2753 (1994)).

¹H NMR (500 MHz, CDCl₃): δ 7.87 (d, 2H, J=8.3 Hz), 7.34 (d, 2H, J=8.5 Hz), 7.14 (d, J=8.7 Hz, 2H), 6.83 (d, J=8.7, 2H), 3.78 (s, 3H), 3.75 (dd, 1H, J=7.2, 4.5 Hz), 2.97 (d, 1H, J=7.2 Hz), 2.44 (s, 3H), 2.39 (d, 1H, J=4.5 Hz)

### N-(p-Tolylsulfonyl)-2-(p-chlorophenyl)aziridine

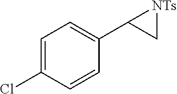 [SMILES: Cc1ccc(S(=O)(=O)N2CC2c2ccc(Cl)cc2)cc1]

Synthesized as previously reported in Ando, T et al., Tetrahedron, 54, 13485-13494 (1998) with spectral data in agreement with literature reported values (Huang, C. Y. et al., J. Am. Chem. Soc., 134, 9541-9544 (2012); Kiyokawa, K. et al., Org. Lett. 15, 4858-4861 (2013); Evans. D. et al., J. Am. Chem. Soc., 116, 2742-2753 (1994)).

¹H NMR (300 MHz, CDCl₃): δ 7.86 (d, 2H, J=8.3 Hz), 7.34 (d, 2H, J=7.9 Hz), 7.26 (d, 2H, J=8.5 Hz), 7.15 (d, 2H, J=8.5 Hz), 3.73 (dd, 1H, J=7.2, 4.4 Hz), 2.98 (d, 1H, J=7.2 Hz), 2.44 (s, 3H), 2.34 (d, 1H, J=4.4 Hz)

### N-(p-Tolylsulfonyl)-2-(m-chlorophenyl)aziridine

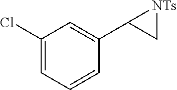 [SMILES: Cc1ccc(S(=O)(=O)N2CC2c2cccc(Cl)c2)cc1]

Synthesized as previously reported in Ando, T et al., Tetrahedron, 54, 13485-13494 (1998) with spectral data in agreement with literature reported values (Craig II, R. A.; et al. Chem. Eur. J., 20, 4806-4813 (2014)).

¹H NMR (400 MHz, CDCl₃): δ 7.87 (d, 2H, J=8.3 Hz), 7.35 (d, 2H, J=7.7 Hz), 7.19-7.26 (m, 3H), 7.12 (dt, 1H, J=6.8, 1.8 Hz), 3.73 (dd, 1H, J=7.2, 4.3 Hz), 2.97 (d, 1H, J=7.2 Hz), 2.44 (s, 3H), 2.35 (d, 1H, J=4.4 Hz)

### N-(p-Tolylsulfonyl)-2-(m-methylphenyl)aziridine

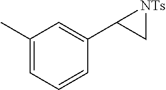 [SMILES: Cc1ccc(S(=O)(=O)N2CC2c2cccc(C)c2)cc1]

Synthesized as previously reported in Ando, T et al., Tetrahedron, 54, 13485-13494 (1998) with spectral data in agreement with literature reported values (Gao, G. Y. et al., Org. Lett., 7, 3191-3193 (2005)).

¹H NMR (400 MHz, CDCl₃): δ 7.87 (d, 2H, J=8.3 Hz), 7.33 (d, 2H, J=8.6 Hz), 7.01-7.20 (m, 4H), 3.74 (dd, 1H, J=7.2, 4.5 Hz), 2.96 (d, 1H, J=7.2 Hz), 2.43 (s, 3H), 2.38 (d, 1H, J=4.5 Hz), 2.30 (s, 3H)

### N-(p-Tolylsulfonyl)-2-(2,4-dimethylphenyl)aziridine

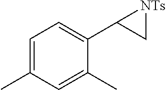 [SMILES: Cc1ccc(S(=O)(=O)N2CC2c2ccc(C)cc2C)cc1]

Synthesized as previously reported in Ando, T et al., Tetrahedron, 54, 13485-13494 (1998).

¹H NMR (400 MHz, CDCl₃): δ 7.90 (d, 2H, J=8.4 Hz), 7.34 (d, 2H, J=8.5 Hz), 6.91-7.00 (m, 3H), 3.84 (dd, 1H, J=7.2, 4.6 Hz), 2.97 (d, 1H, J=7.2 Hz), 2.44 (s, 3H), 2.35 (s, 3H), 2.32 (d, 1H, J=4.6 Hz), 2.28 (s, 3H)

¹³C NMR (101 MHz, CDCl₃): δ 144.72, 137.95, 136.72, 135.15, 130.89, 130.32, 129.84, 128.11, 126.82, 125.98, 39.61, 35.07, 21.75, 21.11, 19.08

HRMS (FAB+): calculated for C₁₇H₂₀NO₂S ([M+H]+): 302.1215. found: 302.1210.

### N-(2-hydroxy-2-phenylpropyl)-4-methylbenzenesulfonamide

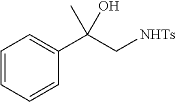

Synthesized as previously reported in Srinivas, B. et al., J. Mol. Catal. A: Chem., 261, 1-5 (2007).

¹H NMR (400 MHz, CDCl₃): δ 7.67 (d, 2H, J=8.3 Hz), 7.24-7.38 (m, 7H), 4.59 (s, 1H), 3.22 (dd, 1H, J=12.8, 8.5 Hz), 3.12 (dd, 1H, J=12.8, 4.8 Hz), 2.42 (s, 3H), 1.56 (s, 3H)

¹³C NMR (101 MHz, CDCl₃): δ 144.87, 143.73, 136.73, 129.93, 128.75, 127.60, 127.19, 124.93, 73.81, 53.99, 27.62, 21.68

HRMS (FAB+): calculated for C₁₆H₂₀NO₃S ([M+H]+): 306.1164. found: 306.1160.

### N-(p-Tolylsulfonyl)-2-(naphthalene-2-yl)aziridine

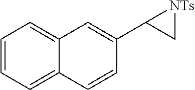 [SMILES: Cc1ccc(S(=O)(=O)N2CC2c2ccc3ccccc3c2)cc1]

Synthesized as previously reported in Ando, T et al., Tetrahedron, 54, 13485-13494 (1998) with spectral data in agreement with literature reported values (Huang, C. Y. et al., J. Am. Chem. Soc., 134, 9541-9544 (2012); Kiyokawa, K. et al., Org. Lett. 15, 4858-4861 (2013); Evans. D. et al., J. Am. Chem. Soc., 116, 2742-2753 (1994)).

¹H NMR (400 MHz, CDCl₃): δ 7.90 (d, 2H, J=8.3 Hz), 7.75-7.81 (m, 3H), 7.73 (s, 1H), 7.45-7.49 (m, 2H), 7.33 (d, 2H, J=8.3 Hz), 7.25-7.30 (m, 1H), 3.93 (dd, 1H, J=7.2, 4.4 Hz), 3.07 (d, 1H, J=7.2 Hz), 2.50 (d, 1H, J=4.5 Hz), 2.42 (s, 3H)

### Materials and Methods

The following paragraphs describe in more detail the materials and methods used in Examples 1-3.

General.

Unless otherwise noted, all chemicals and reagents for reactions were obtained from commercial suppliers (Sigma-Aldrich, VWR, Alfa Aesar) and used without further purification. Silica gel chromatography purifications were carried out using AMD Silica Gel 60, 230-400 mesh. ¹H spectra were recorded on a Varian Inova 300 MHz or Bruker Prodigy 400 MHz instrument in CDCl₃, and are referenced to the residual solvent peak. Synthetic reactions were monitored using thin layer chromatography (Merck 60 gel plates) using an UV-lamp for visualization.

Chromatography.

Analytical high-performance liquid chromatography (HPLC) was carried out using an Agilent 1200 series, and a Kromasil 100 C18 column (4.6×50 mm, 5 μm). Semi-preparative HPLC was performed using an Agilent XDB-C18 (9.4×250 mm, 5 μm). Analytical chiral HPLC was conducted using a supercritical fluid chromatography (SFC) system with isopropanol and liquid CO₂ as the mobile phase. Chiral OB-H and AS-H columns were used to separate aziridine and amido-alcohol enantiomers (4.6×150 mm, 5 μm). Olefins were all commercially available; amido-alcohol and aziridine standards were prepared as reported. % ee was calculated by dividing the major peak area by the sum of the peak areas determined by SFC chromatography

Cloning and Site-Directed Mutagenesis.

pET22b(+) was used as a cloning and expression vector for all enzymes described in this study. Site-directed mutagenesis was performed using a modified QuikChange™ mutagenesis protocol. The PCR products were gel purified, digested with DpnI, repaired using Gibson Mix™, and directed transformed into E. coli strain BL21(DE3).

Determination of P450 Concentration.

Concentration of P450/P411 enzymes for in whole cell experiments was determined from ferrous carbon monoxide binding difference spectra using previously reported extinction coefficients for cysteine-ligated (c=91,000 M⁻¹ cm⁻¹) and serine-ligated enzymes (c=103,000 M⁻¹ cm⁻¹). When purified enzymes were used, concentration of P450/P411 enzymes was accomplished by quantifying the amount of free hemin present in purified protein using the pyridine/hemochrome assay.

Protein Expression and Purification.

Enzymes used in purified protein experiments were expressed in BL21(DE3) E. coli cultures transformed with plasmid encoding P450 or P411 variants. Expression and purification were performed as described except that the shake rate was lowered to 130 RPM during expression (Coelho, P. S., et al. Science, 339, 307 (2013)). Following expression, cells were pelleted and frozen at −20 OC. For purification, frozen cells were resuspended in buffer A (20 mM tris, 20 mM imidazole, 100 mM NaCl, pH 7.5, 4 mL/g of cell wet weight), loaded with 300 μg/ml hemin, and disrupted by sonication (2×1 min, output control 5, 50% duty cycle; Sonicator 3000, Misonix, Inc.). To pellet insoluble material, lysates were centrifuged (20,000×g for 0.5 h at 4° C.). Proteins were expressed in a construct containing a 6×-His tag and were consequently purified using a nickel NTA column (1 mL HisTrap HP, GE Healthcare, Piscataway, N.J.) using an AKTAxpress purifier FPLC system (GE healthcare). P450 or P411 enzymes were then eluted on a linear gradient from 0% buffer B (20 mM tris, 300 mM imidazole, 100 mM NaCl, pH 7.5) to 100% buffer B over 10 column volumes (P450/P411 enzymes elute at around 80 mM imidazole). Fractions containing P450 or P411 enzymes were pooled, concentrated, and subjected to three exchanges of phosphate buffer (0.1 M KPi pH 8.0) to remove excess salt and imidazole. Concentrated proteins were aliquoted, flash-frozen on powdered dry ice, and stored at −20° C. until later use

Reaction Screening in 96-Well Plate Format.

Site-saturation mutagenesis libraries were generated by employing the “22c-trick” method (Kille, S., et al., ACS Synth. Biol., 2, 83-92 (2013)). E. coli libraries were generated and cultured in 300 μL of LB with 100 ug/ml ampicillin and stored as glycerol stocks at −80° C. in 96-well plates. 50 μL of the pre-culture was transferred to a 1000 μL of Hyperbroth using a multichannel pipette. The cultures were incubated at 37° C., 220 rpm, 80% humidity for 3 hours. The plates were cooled on ice for 15 minutes before expression was induced (0.5 mM IPTG, 1 mM 5-aminolevulinic acid final concentration). Expression was conducted at 20° C., 120 rpm, 20 h. The cells were pelleted (3000×g, 5 min) and re-suspended in 40 μL/well GOX solution (14,000 U/ml catalase (Sigma 02071) and 1000 U/ml glucose oxidase (Sigma G7141)). The 96-well plate was transferred to an anaerobic chamber. To this mixture was added 300 μL per well argon sparged reaction buffer (4:1 M9-N: 250 mM glucose in M9-N) was added followed by 4-methylstyrene (300 mM, 10 μL/well) and tosyl azide (100 mM, 10 μL/well). The plate was sealed with aluminum sealing tape, removed from the anaerobic chamber, and shaken at 40 rpm. After 16 hours, the seal was removed and 400 μL of acetonitrile was added to each well. The contents of each well were mixed by pipetting up and down using a multichannel pipette. Then the plate was centrifuged (4000×g, 5 minutes) and 500 μL of the supernatant was transferred to a shallow-well plate for analysis by HPLC.

Typical Procedure for Small-Scale Aziridination Bioconversions Under Anaerobic Conditions Using Whole Cells and Purified Enzymes.

E. coli BL21(DE3) cells containing P450 or P411 enzymes were grown from glycerol stock overnight (37° C., 250 rpm) in 5 ml Luria broth with 0.1 mg mL⁻¹ ampicillin. The preculture was used to inoculate 45 mL of Hyperbroth medium (prepared from AthenaES© powder, 0.1 mg mL¹ ampicillin) in a 125 mL Erlenmeyer flask; this culture was incubated at 37° C., 220 rpm for 2 h and 30 min. After, the cultures were cooled on ice and induced with 0.5 mM IPTG and 1 mM 5-aminolevulinic acid (final concentration). Expression was conducted at room temperature, 120 rpm, 20 h. The cultures were then harvested and resuspended to OD₆₀₀=30 in M9-N. Aliquots of the cell suspension (4 mL) were used for determination of the P450 or P411 expression level after lysis. E. coli cells (OD₆₀₀=30) were made anaerobic by sparging with argon in a sealed 6 mL crimp vial for at least 30 minutes. To a 2 mL crimp vial was then added glucose (250 mM in M9-N, 40 μL) and the GOX solution described previously (20 μL). The headspace of the sealed 2 mL reaction vial was made anaerobic by flushing argon over the solution. Resuspended cells (320 μL), followed by olefin substrate (10 μL, 300 mM in DMSO), then tosyl azide (10 μL, 100 mM in DMSO) were added to 2 mL reaction vial via syringe under continuous flow of argon. Final concentrations of reagents were typically: 2.5 mM tosyl azide, 7.5 mM olefin, 25 mM glucose. The no enzyme control experiment was conducted using E. coli BL21 (DE3) cells containing empty pET22b(+) vector with the same reaction conditions as described above. Purified enzyme reactions were conducted as described previously, using 2.5 mM TsN₃ and 7.5 mM olefin (Farwell, C. C. et al. J. Am. Chem. Soc., 136, 8766-8771 (2014)). Sodium dithionite (5 mM) was used as reductant for reactions with hemin, myoglobin, cytochrome C, CYP119, and P450_{Rhf}. The reactions were shaken on a table-top shake plate (40 rpm) at room temperature for 4 hours. The reactions were quenched by adding acetonitrile (460 μL) and the resulting mixture was transferred to a microcentrifuge tube and centrifuged at 14,000 rpm for 5 minutes. The solution (540 μL) was transferred to an HPLC vial, charged with internal standard (60 μL, 10 mM 1,3,5-trichlorobenzene in acetonitrile), and analyzed by HPLC.

Reactions for chiral HPLC analysis were performed on a 2 mL scale with the same concentration of reagents and using a similar procedure as described above. Briefly, cells containing P450 or P411 enzymes were expressed and resuspended to an OD₆₀₀=30 in M9-N, and then degassed by sparging with argon in a sealed 6 mL crimp vial for at least 30 minutes. To a 6 mL crimp vial was then added glucose (250 mM in M9-N, 200 μL) and the GOX mixture described previously (100 μL). The headspace of the sealed 2 mL reaction vial was made anaerobic by flushing argon over the solution. Resuspended cells (1600 μL), followed by olefin substrate (50 μL, 300 mM in DMSO), then tosyl azide (50 μL, 100 mM in DMSO) were added to 6 mL reaction vial via syringe under continuous flow of argon. Reactions were quenched with 2 mL acetonitrile, extracted with ethyl acetate, dried and resuspended in acetone (200 μL), and purified by C18 semi-preparative HPLC. The purified material was dried, resuspended in acetonitrile, and analyzed by SFC for enantioselectivity.

Determination of Initial Rates.

All initial rate experiments were conducted in an anaerobic chamber. Initial rate measurements were accomplished using 0.2 mol % purified enzymes in 400 μL scale reactions. A sealed 6-mL vial charged with glucose (250 mM, 480 μL), NADPH (100 mM, 480 μL), and potassium phosphate buffer (0.1 M, pH=8.0, 3240 μL) was sparged for at least 30 minutes with argon. After the degassing was complete, the reaction solution, 2-mL vials charged with GOX solution (20 μL), and purified protein (250 μM in potassium phosphate buffer), kept on ice, were brought into the anaerobic chamber. The reaction solution (350 μL) was added to each 2-mL vial and allowed to equilibrate in the anaerobic chamber for 30 minutes. Reaction vials were then placed on a shaker (40 rpm), charged with 10 μL purified protein (250 μM in potassium phosphate buffer) and 4-methyl styrene substrate (10 μL, 300 mM in DMSO) followed by tosyl azide (10 μL, 100 mM in DMSO). Reactions were set up in duplicate and products quantified at 1-2 minute intervals by quenching with acetonitrile (460 μL). The resulting mixture was removed from the anaerobic chamber, transferred to a microcentrifuge tube and centrifuged at 14,000 rpm for 5 minutes. The solution (540 μL) was transferred to an HPLC vial, charged with internal standard (60 μL, 10 mM 1,3,5-trichlorobenzene in acetonitrile), and analyzed by HPLC. The rates of aziridination and azide reduction for different enzyme variants are presented in FIG. 6. The rate of azide reduction was determined in the presence of olefin 3 (7.5 mM). Initial rates are plotted for individual enzymes in FIG. 7 A-C.

Assignment of Absolute Stereochemistry.

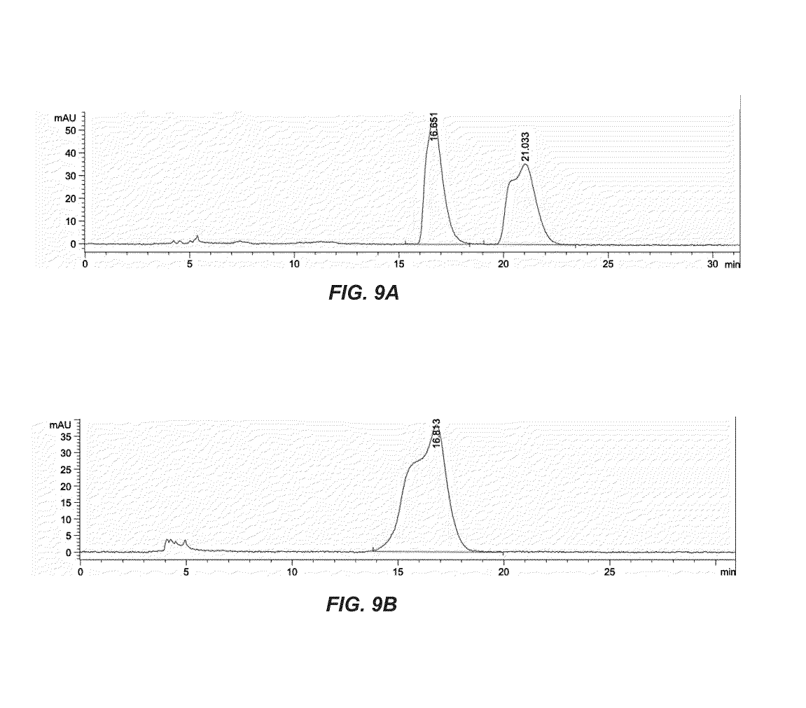

Absolute stereochemistry of enzymatically produced aziridine 6 was assigned by chiral HPLC analysis and optical rotation. In particular, absolute stereochemistry of 6 was previously assigned by chiral HPLC using Chiracel OJ column (isopropanol/n-hexane mobile phase), with (S)-6 the earlier eluting enantiomer (Takeda Y., J. Am. Chem. Soc., 136, 8544-7 (2014)). Analytically enantiopure 6 produced by P-I263F-A328V-L437V was subjected to the same chiral HPLC conditions and observed to be the earlier eluting enantiomer (FIGS. 9A-B), leading to an assignment of (S)-6. Further support for this assignment came from measuring optical rotation. The optical rotation values for enantiomers of 7 have been previously reported (R)-6 [α_{D}²⁵]−80.25 (c=0.8, CHCl₃) and (S)-6 [α_{D}²⁰]+26.7 (c=0.7, CHCl₃) (Alonso, D. A., et al., J. Org. Chem., 63, 9455-9461 (1998); Wang, X. et al., Chem. Eur. J., 12, 4568-4575 (2006)). Optical rotation measurement of analytically enantiopure 6 produced by P-I263F-A328V-L437V gave [α_{D}²⁵]+80.2 (c=1.2, CHCl₃), revealing it to be (S)-6. Similarly, the optical rotation of P-I263F-A328V-L437V produced 4 (analytically enantiopure) was measured to be [α_{D}²⁵]+106.1 (c=0.45, CHCl₃). By analogy, the configuration of enzymatically preferred (+)-4 is assigned as (S)-4.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### Informal Sequence Listing

| CYP102A1 | |
|---|---|
| Cytochrome P450 (BM3) | |
| Bacillus megaterium | |
| GenBank Accession No. AAA87602 | |
| >gi\|142798\|gb\|AAA87602.1\| cytochrome P-450:NADPH-P-450 reductase | |
| precursor [Bacillus megaterium] | |
| SEQ ID NO: 1 | |
| TIKEMPQPK TFGELKNLPL LNTDKPVQAL MKIADELGEI FKFEAPGRVT RYLSSQRLIK | |
| EACDESRFDK NLSQALKFVR DFAGDGLFTS WTHEKNWKKA HNILLPSFSQ QAMKGYHAMM | |
| VDIAVQLVQK WERLNADEHI EVPEDMTRLT LDTIGLCGFN YRFNSFYRDQ PHPFITSMVR | |
| ALDEAMNKLQ RANPDDPAYD ENKRQFQEDI KVMNDLVDKI IADRKASGEQ SDDLLTHMLN | |
| GKDPETGEPL DDENIRYQII TFLIAGHETT SGLLSFALYF LVKNPHVLQK AAEEAARVLV | |
| DPVPSYKQVK QLKYVGMVLN EALRLWPTAP AFSLYAKEDT VLGGEYPLEK GDELMVLIPQ | |
| LHRDKTIWGD DVEEFRPERF ENPSAIPQHA FKPFGNGQRA CIGQQFALHE ATLVLGMMLK | |
| HFDFEDHTNY ELDIKETLTL KPEGFVVKAK SKKIPLGGIP SPSTEQSAKK VRKKAENAHN | |
| TPLLVLYGSN MGTAEGTARD LADIAMSKGF APQVATLDSH AGNLPREGAV LIVTASYNGH | |
| PPDNAKQFVD WLDQASADEV KGVRYSVFGC GDKNWATTYQ KVPAFIDETL AAKGAENIAD | |
| RGEADASDDF EGTYEEWREH MWSDVAAYFN LDIENSEDNK STLSLQFVDS AADMPLAKMH | |
| GAFSTNVVAS KELQQPGSAR STRHLEIELP KEASYQEGDH LGVIPRNYEG IVNRVTARFG | |
| LDASQQIRLE AEEEKLAHLP LAKTVSVEEL LQYVELQDPV TRTQLRAMAA KTVCPPHKVE | |
| LEALLEKQAY KEQVLAKRLT MLELLEKYPA CEMKFSEFIA LLPSIRPRYY SISSSPRVDE | |
| KQASITVSVV SGEAWSGYGE YKGIASNYLA ELQEGDTITC FISTPQSEFT LPKDPETPLI | |
| MVGPGTGVAP FRGFVQARKQ LKEQGQSLGE AHLYFGCRSP HEDYLYQEEL ENAQSEGIIT | |
| LHTAFSRMPN QPKTYVQHVM EQDGKKLIEL LDQGAHFYIC GDGSQMAPAV EATLMKSYAD | |
| VHQVSEADAR LWLQQLEEKG RYAKDVWAG | |
| CYP102A1 | |
| B. megaterium | |
| >gi\|281191140\|gb\|ADA57069.1\| NADPH-cytochrome P450 reductase 102A1V9 | |
| [Bacillus megaterium] | |
| SEQ ID NO: 2 | |
| MTIKEMPQPKTFGELKNLPLLNTDKPIQTLMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HEDFEDHTNYELDIKETLTLKPEGFVVKAKSKQIPLGGIPSPSREQSAKKERKTVENAHNTPLLVLYGSN | |
| MGTAEGTARDLADIAMSKGFAPQVATLDSHAGNLPREGAVLIVTASYNGHPPDNAKEFVDWLDQASADEV | |
| KGVRYSVEGCGDKNWATTYQKVPAFIDETLAAKGAENIAERGEADASDDFEGTYEEWREHMWSDLAAYEN | |
| LDIENSEENASTLSLQFVDSAADMPLAKMHRAFSANVVASKELQKPGSARSTRHLEIELPKEASYQEGDH | |
| LGVIPRNYEGIVNRVATREGLDASQQIRLEAFEEKLAHLPLGKTVSVEELLQYVELQDPVTRTQLRAMAA | |
| KTVCPPHKVELEVLLEKQAYKEQVLAKRLTMLELLEKYPACEMEFSEFIALLPSMRPRYYSISSSPRVDE | |
| KQASITVSVVSGEAWSGYGEYKGIASNYLANLQEGDTITCFVSTPQSGFTLPKGPETPLIMVGPGTGVAP | |
| FRGFVQARKQLKEQGQSLGEAHLYFGCRSPHEDYLYQKELENAQNEGIITLHTAFSRVPNQPKTYVQHVM | |
| EQDGKKLIELLDQGAHFYICGDGSQMAPDVEATLMKSYAEVHQVSEADARLWLQQLEEKGRYAKDVWAG | |
| CYP102A1 | |
| B. megaterium | |
| >gi\|281191138\|gb\|ADA57068.1\| NADPH-cytochrome P450 reductase 102A1V10 | |
| [Bacillus megaterium] | |
| SEQ ID NO: 3 | |
| MTIKEMPQPKTFGELKNLPLLNTDKPIQTLMKIADELGEIFKFEAPGRVTRYLSSQRLIKEACDESRFDK | |
| NLSQALKFVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAFEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HFDFEDHTNYELDIKETLTLKPEGFVVKAKSKQIPLGGIPSPSREQSAKKERKTVENAHNTPLLVLYGSN | |
| MGTAEGTARDLADIAMSKGFAPQVATLDSHAGNLPREGAVLIVTASYNGHPPDNAKEFVDWLDQASADEV | |
| KGVRYSVFGCGDKNWATTYQKVPAFIDETFAAKGAENIAERGEADASDDFEGTYEEWREHMWSDLAAYFN | |
| LDIENSEENASTLSLQFVDSAADMPLAKMHRAFSANVVASKELQKPGSARSTRHLEIELPKEASYQEGDH | |
| LGVIPRNYEGIVNRVATRFGLDASQQIRLEAFEEKLAHLPLGKTVSVEELLQYVELQDPVTRTQLRAMAA | |
| KTVCPPHKVELEVLLEKQAYKEQVLAKRLTMLELLEKYPACEMEFSEFIALLPSMRPRYYSISSSPRVDE | |
| KQASITVSVVSGEAWSGYGEYKGIASNYLANLQEGDTITCFVSTPQSGFTLPKGPETPLIMVGPGTGVAP | |
| FRGFVQARKQLKEQGQSLGEAHLYFGCRSPHEDYLYQKELENAQNEGIITLHTAFSRVPNQPKTYVQHVM | |
| EQDGKKLIELLDQGAHFYICGDGSQMAPDVEATLMKSYAEVHQVSEADARLWLQQLEEKGRYAKDVWAG | |
| CYP102A1 | |
| B. megaterium | |
| >gi\|281191126\|gb\|ADA57062.1\| NADPH-cytochrome P450 reductase 102A1V4 | |
| [Bacillus megaterium] | |
| SEQ ID NO: 4 | |
| MTIKEMPQPKTFGELKNLPLLNTDKPIQTLMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEATRVLVDPVPSYKQVKQLKYVGMVLNEALRLWPTAPAFSLYAKEDTVLGGEYPLEK | |
| GDELMVLIPQLHRDKTIWGEDVEEFRPERFENPSAIPQHAFKPFGNGQRACIGQQFALHEATLVLGMMLK | |
| HEDFEDHTNYELDIKETLTLKPEGFVVKAKSKKIPLGGIPSPSTEQSAKKVRKKVENAHNTPLLVLYGSN | |
| MGTAEGTARDLADIAMSKGFAPQVATLDSHAGNLPREGAVLIVTASYNGHPPDNAKQFVDWLDQASADDV | |
| KGVRYSVEGCGDKNWATTYQKVPAFIDETLAAKGAENIADRGEADASDDFEGTYEEWREHMWSDVAAYEN | |
| LDIENSEDNKSTLSLQFVDSAADMPLAKMHGAFSANVVASKELQQPGSERSTRHLEIALPKEASYQEGDH | |
| LGVIPRNYEGIVNRVTAREGLDASQQIRLEAFEEKLAHLPLGKTVSVEELLQYVELQDPVTRTQLRAMAA | |
| KTVCPPHKVELEALLEKQAYKEQVLAKRLTMLELLEKYPACEMEFSEFIALLPSIRPRYYSISSSPRVDE | |
| KQASITVSVVSGEAWSGYGEYKGIASNYLANLQEGDTITCFVSTPQSGFTLPKDSETPLIMVGPGTGVAP | |
| FRSFVQARKQLKEQGQSLGEAHLYFGCRSPHEDYLYQEELENAQNEGIITLHTAFSRVPNQPKTYVQHVM | |
| EQDGKKLIELLDQGAHFYICGDGSQMAPDVEATLMKSYADVYEVSEADARLWLQQLEEKGRYAKDVWAG | |
| CYP102A1 | |
| B. megaterium | |
| >gi\|281191124\|gb\|ADA57061.1\| NADPH-cytochrome P450 reductase 102A1V8 | |
| [Bacillus megaterium] | |
| SEQ ID NO: 5 | |
| MTIKEMPQPKTFGELKNLPLLNTDKPIQTLMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HEDFEDHTNYELDIKETLTLKPEGFVVKAKSKQIPLGGIPSPSREQSAKKERKTVENAHNTPLLVLYGSN | |
| MGTAEGTARDLADIAMSKGFAPRVATLDSHAGNLPREGAVLIVTASYNGHPPDNAKEFVDWLDQASADEV | |
| KGVRYSVEGCGDKNWATTYQKVPAFIDETLAAKGAENIAERGEADASDDFEGTYEEWREHMWSDLAAYEN | |
| LDIENSEENASTLSLQFVDSAADMPLAKMHRAFSANVVASKELQKPGSARSTRHLEIELPKEASYQEGDH | |
| LGVIPRNYEGIVNRVATREGLDASQQIRLEAFEEKLAHLPLGKTVSVEELLQYVELQDPVTRTQLRAMAA | |
| KTVCPPHKVELEVLLEKQAYKEQVLAKRLTMLELLEKYPACEMEFSEFIALLPSMRPRYYSISSSPRVDE | |
| KQASITVSVVSGEAWSGYGEYKGIASNYLANLQEGDTITCFVSTPQSGFTLPKGPETPLIMVGPGTGVAP | |
| FRGFVQARKQLKEQGQSLGEAHLYFGCRSPHEDYLYQKELENAQNEGIITLHTAFSRVPNQPKTYVQHVM | |
| EQDGKKLIELLDQGAHFYICGDGSQMAPDVEATLMKSYAEVHQVSEADARLWLQQLEEKGRYAKDVWAG | |
| CYP102A1 | |
| B. megaterium | |
| >gi\|281191120\|gb\|ADA57059.1\| NADPH-cytochrome P450 reductase 102A1V3 | |
| [Bacillus megaterium] | |
| SEQ ID NO: 6 | |
| MTIKEMPQPKTFGELKNLPLLNTDKPVQALMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLVQKWERLNADEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HEDFEDHTNYELDIKETLTLKPEGFVVKAKSKKIPLGGIPSPSTEQSAKKVRKKVENAHNTPLLVLYGSN | |
| MGTAEGTARDLADIAMSKGFAPQVATLDSHAGNLPREGAVLIVTASYNGHPPDNAKQFVDWLDQASADDV | |
| KGVRYSVEGCGDKNWATTYQKVPAFIDETLAAKGAENIADRGEADASDDFEGTYEEWREHMWSDVAAYEN | |
| LDIENSEDNKSTLSLQFVDSAADMPLAKMHGAFSANVVASKELQQLGSERSTRHLEIALPKEASYQEGDH | |
| LGVIPRNYEGIVNRVTAREGLDASQQIRLEAFEEKLAHLPLGKTVSVEELLQYVELQDPVTRTQLRAMAA | |
| KTVCPPHKVELEALLEKQAYKEQVLAKRLTMLELLEKYPACEMEFSEFIALLPSISPRYYSISSSPHVDE | |
| KQASITVSVVSGEAWSGYGEYKGIASNYLANLQEGDTITCFVSTPQSGFTLPKDSETPLIMVGPGTGVAP | |
| FRGFVQARKQLKEQGQSLGEAHLYFGCRSPHEDYLYQEELENAQNEGIITLHTAFSRVPNQPKTYVQHVM | |
| ERDGKKLIELLDQGAHFYICGDGSQMAPDVEATLMKSYADVYEVSEADARLWLQQLEEKGRYAKDVWAG | |
| CYP102A1 | |
| B. megaterium | |
| >gi\|281191118\|gb\|ADA57058.1\| NADPH-cytochrome P450 reductase 102A1V7 | |
| [Bacillus megaterium] | |
| SEQ ID NO: 7 | |
| MTIKEMPQPKTFGELKNLPLLNTDKPIQTLMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HEDFEDHTNYELDIKETLTLKPEGFVVKAKSKQIPLGGIPSPSREQSAKKERKTVENAHNTPLLVLYGSN | |
| MGTAEGTARDLADIAMSKGFAPQVATLDSHAGNLPPEGAVLIVTASYNGHPPDNAKEFVDWLDQASADEV | |
| KGVRYSVEGCGDKNWATTYQKVPAFIDETLAAKGAENIAERGEADASDDFEGTYEEWREHMWSDLAAYEN | |
| LDIENSEENASTLSLQFVDSAADMPLAKMHRAFSANVVASKELQKPGSARSTRHLEIELPKEASYQEGDH | |
| LGVIPRNYEGIVNRVATREGLDASQQIRLEAFEEKLAHLPLGKTVSVEELLQYVELQDPVTRTQLRAMAA | |
| KTVCPPHKVELEVLLEKQAYKEQVLAKRLTMLELLEKYPACEMEFSEFIALLPSMRPRYYSISSSPRVDE | |
| KQASITVSVVSGEAWSGYGEYKGIASNYLANLQEGDTITCFVSTPQSGFTLPKGPETPLIMVGPGTGVAP | |
| FRGFVQARKQLKEQGQSLGEAHLYFGCRSPHEDYLYQKELENAQNEGIITLHTAFSRVPNEPKTYVQHVM | |
| EQDGKKLIELLDQGAHFYICGDGSQMAPDVEATLMKSYAEVHQVSEADARLWLQQLEEKGRYAKDVWAG | |
| CYP102A1 | |
| B. megaterium | |
| >gi\|281191112\|gb\|ADA57055.1\| NADPH-cytochrome P450 reductase 102A1V2 | |
| [Bacillus megaterium] | |
| SEQ ID NO: 8 | |
| MTIKEMPQPKTFGELKNLPLLNTDKPIQTLMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEATRVLVDPVPSYKQVKQLKYVGMVLNEALRLWPTAPAFSLYAKEDTVLGGEYPLEK | |
| GDELMVLIPQLHRDKTIWGEDVEEFRPERFENPSAIPQHAFKPFGNGQRACIGQQFALHEATLVLGMMLK | |
| HEDFEDHTNYELDIKETLTLKPEGFVVKAKSKKIPLGGIPSPSTEQSAKKVRKKVENAHNTPLLVLYGSN | |
| MGTAEGTARDLADIAMSKGFAPQVATLDSHAGNLPREGAVLIVTASYNGHPPDNAKQFVDWLDQASADDV | |
| KGVRYSVEGCGDKNWATTYQKVPAFIDETLAAKGAENIADRGEADASDDFEGTYEEWREHMWSDVAAYEN | |
| LDIENSEDNKSTLSLQFVDSAADMPLAKMHGAFSANVVASKELQQLGSERSTRHLEIALPKEASYQEGDH | |
| LGVIPRNYEGIVNRVTARFGLDASQQIRLEAFEEKLAHLPLGKTVSVEELLQYVELQDPVTRTQLRAMAA | |
| KTVCPPHKVELEALLEKQAYKEQVLAKRLTMLELLEKYPACEMEFSEFIALLPSISPRYYSISSSPHVDE | |
| KQASITVSVVSGEAWSGYGEYKGIASNYLANLQEGDTITCFVSTPQSGFTLPKDSETPLIMVGPGTGVAP | |
| FRGFVQARKQLKEQGQSLGEAHLYFGCRSPHEDYLYQEELENAQNEGIITLHTAFSRVPNQPKTYVQHVM | |
| ERDGKKLIELLDQGAHFYICGDGSQMAPDVEATLMKSYADVYEVSEADARLWLQQLEEKGRYAKDVWAG | |
| CYP102A1 | |
| B. megaterium | |
| >gi\|269315992\|gb\|ACZ37122.1\| cytochrome P450:NADPH P450 reductase | |
| [Bacillus megaterium] | |
| SEQ ID NO: 9 | |
| MTIKEMPQPKTFGELKNLPLLNTDKPIQTLMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNTDEHI | |
| EVPEDMTRLTLDTIGLCGFNYRFNSFYRDQPHPFITSMVRALDEAMNKLQRANPDDPAYDENKRQFQEDI | |
| KVMNDLVDKIIADRKASGEQSDDLLTHMLNGKDPETGEPLDDENIRYQIITFLIAGHETTSGLLSFALYF | |
| LVKNPHVLQKAAEEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HEDFEDHTNYELDIKETLTLKPEGFVVKAKSKQIPLGGIPSPSREQSAKKERKTVENAHNTPLLVLYGSN | |
| MGTAEGTARDLADIAMSKGFAPQVATLDSHAGNLPREGAVLIVTASYNGHPPDNAKEFVDWLDQASADEV | |
| KGVRYSVEGCGDKNWATTYQKVPAFIDETLAAKGAENIAERGEADASDDFEGTYEEWREHMWSDLAAYEN | |
| LDIENSEENASTLSLQFVDSAADMPLAKMHRAFSANVVASKELQKPGSARSTRHLEIELPKEASYQEGDH | |
| LGVIPRNYEGIVNRVATREGLDASQQIRLEAFEEKLAHLPLGKTVSVEELLQYVELQDPVTRTQLRAMAA | |
| KTVCPPHKVELEVLLEKQAYKEQVLAKRLTMLELLEKYPACEMEFSEFIALLPSMRPRYYSISSSPRVDE | |
| KQASITVSVVSGEAWSGYGEYKGIASNYLANLQEGDTITCFVSTPQSGFTLPKGPETPLIMVGPGTGVAP | |
| FRGFVQARKQLKEQGQSLGEAHLYFGCRSPHEDYLYQKELENAQNEGIITLHTAFSRVPNQPKTYVQHVM | |
| EQDGKKLIELLDQGAHFYICGDGSQMAPDVEATLMKSYAEVHQVSEADARLWLQQLEEKGRYAKDVWAG | |
| CYP102A1 | |
| B. megaterium | |
| >gi\|281191116\|gb\|ADA57057.1\| NADPH-cytochrome P450 reductase 102A1V6 | |
| [Bacillus megaterium] | |
| SEQ ID NO: 10 | |
| MTIKEMPQPKTFGELKNLPLLNTDKPVQALMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNADEHI | |
| EVPEDMTRLTLDTIGLCGFNYRFNSFYRDQPHPFITSMVRALDEAMNKLQRANPDDPAYDENKRQFQDDI | |
| KVMNDLVDKIIADRKASGEQSDDLLTHMLNGKDPETGEPLDDENIRYQIITFLIAGHETTSGLLSFALYF | |
| LVKNPHVLQKAAEEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HEDFEDHTNYELDIKETLTLKPEGFVVKAKSKQIPLGGIPSPSREQSAKKERKTVENAHNTPLLVLYGSN | |
| MGTAEGTARDLADIAMSKGFAPQVATLDSHAGNLPREGAVLIVTASYNGHPPDNAKQFVDWLDQASADEV | |
| KGVRYSVEGCGDKNWATTYQKVPAFIDETLSAKGAENIAERGEADASDDFEGTYEEWREHMWSDLAAYEN | |
| LNIENSEDNASTLSLQFVDSAADMPLAKMHGAFSANVVASKELQQPGSARSTRHLEIELPKEASYQEGDH | |
| LGVIPRNYEGIVNRVTTREGLDASQQIRLEAFEEKLAHLPLGKTVSVEELLQYVELQDPVTRTQLRAMAA | |
| KTVCPPHKVELEALLEKQAYKEQVLTKRLTMLELLEKYPACEMEFSEFIALLPSMRPRYYSISSSPRVDE | |
| KQASITVSVVSGEAWSGYGEYKGIASNYLAELQEGDTITCFVSTPQSGFTLPKDPETPLIMVGPGTGVAP | |
| FRGFVQARKQLKEQGQSLGEAHLYFGCRSPHEDYLYQEELENAQNEGIITLHTAFSRVPNQPKTYVQHVV | |
| EQDGKKLIELLDQGAHFYICGDGSQMAPDVEATLMKSYAEVHKVSEADARLWLQQLEEKSRYAKDVWAG | |
| CYP102A1 | |
| B. megaterium | |
| >gi\|281191114\|gb\|ADA57056.1\| NADPH-cytochrome P450 reductase 102A1V5 | |
| [Bacillus megaterium] | |
| SEQ ID NO: 11 | |
| MTIKEMPQPKTFGELKNLPLLNTDKPVQALMKIADELGEIFKFEAPGRVTRYLSSQRLIKEACDESRFDK | |
| NLSQALKEVRDFAGDGLFTSWTHEKNWKKAHNILLPSFSQQAMKGYHAMMVDIAVQLIQKWERLNADEHI | |
| EVPEDMTRLTLDTIGLCGFNYRFNSFYRDQPHPFITSMVRALDEAMNKLQRANPDDPAYDENKRQFQDDI | |
| KVMNDLVDKIIADRKASGEQSDDLLTHMLNGKDPETGEPLDDENIRYQIITFLIAGHETTSGLLSFALYF | |
| LVKNPHVLQKAAFEAARVLVDPVPSYKQVKQLKYVGMVLNEALRLWPTAPAFSLYAKEDTVLGGEYPLEK | |
| GDELMVLIPQLHRDKTIWGDDVEEFRPERFENPSAIPQHAFKPFGNGQRACIGQQFALHEATLVLGMMLK | |
| HFDFEDHTNYELDIKETLTLKPEGFVVKAKSKQIPLGGIPSPSREQSAKKERKTVENAHNTPLLVLYGSN | |
| MGTAEGTARDLADIAMSKGFAPQVATLDSHAGNLPREGAVLIVTASYNGHPPDNAKQFVDWLDQASADEV | |
| KGVRYSVFGCGDKNWATTYQKVPAFIDETLSAKGAENIAERGEADASDDFEGTYEEWREHMWSDLAAYFN | |
| LNIENSEDNASTLSLQFVDSAADMPLAKMHGAFSANVVASKELQQPGSARSTRHLEIELPKEASYQEGDH | |
| LGVIPRNYEGIVNRVTTRFGLDASQQIRLEAFEEKLAHLPLGKTVSVEELLQYVELQDPVTRTQLRAMAA | |
| KTVCPPHKVELEALLEKQAYKEQVLTKRLTMLELLEKYPACEMEFSEFIALLPSMRPRYYSISSSPRVDE | |
| KQASITVSVVSGEAWSGYGEYKGIASNYLAELQEGDTITCFVSTPQSGFTLPKDPETPLIMVGPGTGVAP | |
| FRGFVQARKQLKEQGQSLGEAHLYFGCRSPHEDYLYQEELENAQNEGIITLHTAFSRVPNQPKTYVQHVV | |
| EQDGKKLIELLDQGAHFYICGDGSQMAPDVEATLMKSYAEVHKVSEADARLWLQQLEEKSRYAKDVWAG | |
| CYP153A6 | |
| Mycobacterium sp. HXN-1500 | |
| GenBank Accession No.: CAH04396 | |
| >gi\|51997117\|emb\|CAH04396.1\| cytochrome P450 alkane hydroxylase | |
| [Mycobacterium sp. HXN-1500] | |
| SEQ ID NO: 12 | |
| 1 | MTEMTVAASD ATNAAYGMAL EDIDVSNPVL FRDNTWHPYF KRLREEDPVH YCKSSMFGPY |
| 61 | WSVTKYRDIM AVETNPKVFS SEAKSGGITI MDDNAAASLP MFIAMDPPKH DVQRKTVSPI |
| 121 | VAPENLATME SVIRQRTADL LDGLPINEEF DWVHRVSIEL TTKMLATLFD FPWDDRAKLT |
| 181 | RWSDVTTALP GGGIIDSEEQ RMAELMECAT YFTELWNQRV NAEPKNDLIS MMAHSESTRH |
| 241 | MAPEEYLGNI VLLIVGGNDT TRNSMTGGVL ALNEFPDEYR KLSANPALIS SMVSEIIRWQ |
| 301 | TPLSHMRRTA LEDIEFGGKH IRQGDKVVMW YVSGNRDPEA IDNPDTFIID RAKPRQHLSF |
| 361 | GFGIHRCVGN RLAELQLNIL WEEILKRWPD PLQIQVLQEP TRVLSPFVKG YESLPVRINA |
| CYP5013C2 | |
| Tetrahymena thermophile | |
| GenBank Accession No.: ABY59989 | |
| >gi\|164519863\|gb\|ABY59989.1\| cytochrome P450 monooxygenase CYP5013C2 | |
| [Tetrahymena thermophila] | |
| SEQ ID NO: 13 | |
| 1 | MIFELILIAV ALFAYFKIAK PYFSYLKYRK YGKGFYYPIL GEMIEQEQDL KQHADADYSV |
| 61 | HHALDKDPDQ KLFVTNLGTK VKLRLIEPEI IKDFFSKSQY YQKDQTFIQN ITRFLKNGIV |
| 121 | FSEGNTWKES RKLFSPAFHY EYIQKLTPLI NDITDTIFNL AVKNQELKNF DPIAQIQEIT |
| 181 | GRVIIASFFG EVIEGEKFQG LTIIQCLSHI INTLGNQTYS IMYFLFGSKY FELGVTEEHR |
| 241 | KFNKFIAEFN KYLLQKIDQQ IEIMSNELQT KGYIQNPCIL AQLISTHKID EITRNQLFQD |
| 301 | FKTFYIAGMD TTGHLLGMTI YYVSQNKDIY TKLQSEIDSN TDQSAHGLIK NLPYLNAVIK |
| 361 | ETLRYYGPGN ILFDRIAIKD HELAGIPIKK GTIVTPYAMS MQRNSKYYQD PHKYNPSRWL |
| 421 | EKQSSDLHPD ANIPFSAGQR KCIGEQLALL EARIILNKFI KMFDFTCPQD YKLMMNYKFL |
| 481 | SEPVNPLPLQ LTLRKQ |
| Nonomuraea dietziae | |
| >gi\|445067389\|gb\|AGE14547.1\| cytochrome P450 hydroxylase sb8 | |
| [Nonomuraea dietziae] | |
| GenBank Accession No.: AGE14547 | |
| SEQ ID NO: 14 | |
| VNIDLVDQDHYATEGPPHEQMRWLREHAPVYWHEGEPGFWAVTRHEDVVHVSRHSDLESSARRLALFNEMPEEQREL | |
| QRMMMLNQDPPEHTRRRSLVNRGFTPRTIRALEQHIRDICDDLLDQCSGEGDFVTDLAAPLPLYVICELLGAPVADR | |
| DKIFAWSNRMIGAQDPDYAASPEEGGAAAMEVYAYASELAAQRRAAPRDDIVTKLLQSDENGESLTENEFELFVLLL | |
| VVAGNETTRNAASGGMLTLFEHPDQWDRLVADPSLAATAADEIVRWVSPVNLFRRTATADLTLGGQQVKADDKVVVF | |
| YSSANRDASVESDPEVEDIGRSPNPHIGEGGGGAHFCLGNHLAKLELRVLFEQLARREPRMRQTGEARRLRSNFING | |
| IKTLPVTLG | |
| CYP2R1 | |
| Homo sapiens | |
| GenBank Accession No.: NP 078790 | |
| >gi\|45267826\|ref\|NP_078790.2\| vitamin D 25-hydroxylase [Homo sapiens] | |
| SEQ ID NO: 15 | |
| 1 | MWKLWRAEEG AAALGGALFL LLFALGVRQL LKQRRPMGFP PGPPGLPFIG NIYSLAASSE |
| 61 | LPHVYMRKQS QVYGEIFSLD LGGISTVVLN GYDVVKECLV HQSEIFADRP CLPLFMKMTK |
| 121 | MGGLLNSRYG RGWVDHRRLA VNSFRYFGYG QKSFESKILE ETKFFNDAIE TYKGRPFDFK |
| 181 | QLITNAVSNI TNLIIFGERF TYEDTDFQHM IELFSENVEL AASASVFLYN AFPWIGILPF |
| 241 | GKHQQLFRNA AVVYDFLSRL IEKASVNRKP QLPQHFVDAY LDEMDQGKND PSSTFSKENL |
| 301 | IFSVGELIIA GTETTTNVLR WAILFMALYP NIQGQVQKEI DLIMGPNGKP SWDDKCKMPY |
| 361 | TEAVLHEVLR FCNIVPLGIF HATSEDAVVR GYSIPKGTTV ITNLYSVHFD EKYWRDPEVF |
| 421 | HPERFLDSSG YFAKKEALVP FSLGRRHCLG EHLARMEMFL FFTALLQRFH LHFPHELVPD |
| 481 | LKPRLGMTLQ PQPYLICAER R |
| CYP2R1 | |
| Macca mulatta | |
| GenBank Accession No.: NP 001180887 | |
| >gi\|302565346\|ref\|NP_001180887.1\| vitamin D 25-hydroxylase | |
| [Macaca mulatta] | |
| SEQ ID NO: 16 | |
| 1 | MWKLWGGEEG AAALGGALFL LLFALGVRQL LKLRRPMGFP PGPPGLPFIG NIYSLAASAE |
| 61 | LPHVYMRKQS QVYGEIFSLD LGGISTVVLN GYDVVKECLV HQSGIFADRP CLPLFMKMTK |
| 121 | MGGLLNSRYG QGWVEHRRLA VNSFRYFGYG QKSFESKILE ETKFFTDAIE TYKGRPFDFK |
| 181 | QLITSAVSNI TNLIIFGERF TYEDTDFQHM IELFSENVEL AASASVFLYN AFPWIGILPF |
| 241 | GKHQQLFRNA SVVYDFLSRL IEKASVNRKP QLPQHFVDAY FDEMDQGKND PSSTFSKENL |
| 301 | IFSVGELIIA GTETTTNVLR WAILFMALYP NIQGQVQKEI DLIMGPNGKP SWDDKFKMPY |
| 361 | TEAVLHEVLR FCNIVPLGIF HATSEDAVVR GYSIPKGTTV ITNLYSVHFD EKYWRDPEVF |
| 421 | HPERFLDSSG YFAKKEALVP FSLGRRHCLG EQLARMEMFL FFTALLQRFH LHFPHELVPD |
| 481 | LKPRLGMTLQ PQPYLICAER R |
| CYP2R1 | |
| Canis familiaris | |
| GenBank Accession No.: XP_854533 | |
| >gi\|73988871\|ref\|XP_854533.11 PREDICTED: vitamin D 25-hydroxylase | |
| [Canis lupus familiaris] | |
| SEQ ID NO: 17 | |
| 1 | MRGPPGAEAC AAGLGAALLL LLFVLGVRQL LKQRRPAGFP PGPSGLPFIG NIYSLAASGE |
| 61 | LAHVYMRKQS RVYGEIFSLD LGGISAVVLN GYDVVKECLV HQSEIFADRP CLPLFMKMTK |
| 121 | MGGLLNSRYG RGWVDHRKLA VNSFRCFGYG QKSFESKILE ETNFFIDAIE TYKGRPFDLK |
| 181 | QLITNAVSNI TNLIIFGERF TYEDTDFQHM IELFSENVEL AASASVFLYN AFPWIGIIPF |
| 241 | GKHQQLFRNA AVVYDFLSRL IEKASINRKP QSPQHFVDAY LNEMDQGKND PSCTFSKENL |
| 301 | IFSVGELIIA GTETTTNVLR WAILFMALYP NIQGQVQKEI DLIMGPTGKP SWDDKCKMPY |
| 361 | TEAVLHEVLR FCNIVPLGIF HATSEDAVVR GYSIPKGTTV ITNLYSVHFD EKYWRNPEIF |
| 421 | YPERFLDSSG YFAKKEALVP FSLGKRHCLG EQLARMEMFL FFTALLQRFH LHFPHGLVPD |
| 481 | LKPRLGMTLQ PQPYLICAER R |
| CYP2R1 | |
| Mus musculus | |
| GenBank Accession No.: AAI08963 | |
| >gi\|80477959\|gb\|AAI08963.1\| Cyp2r1 protein [Mus musculus] | |
| SEQ ID NO: 18 | |
| 1 | MGDEMDQGQN DPLSTFSKEN LIFSVGELII AGTETTTNVL RWAILFMALY PNIQGQVHKE |
| 61 | IDLIVGHNRR PSWEYKCKMP YTEAVLHEVL RFCNIVPLGI FHATSEDAVV RGYSIPKGTT |
| 121 | VITNLYSVHF DEKYWKDPDM FYPERFLDSN GYFTKKEALI PFSLGRRHCL GEQLARMEMF |
| 181 | LFFTSLLQQF HLHFPHELVP NLKPRLGMTL QPQPYLICAE RR |
| CYP152A6 | |
| Bacillus halodurans C-125 | |
| GenBank Accession No.: NP\|242623 | |
| >gi\|15614320\|ref\|NP\|242623.11 fatty acid alpha hydroxylase | |
| [Bacillus halodurans C-125] | |
| SEQ ID NO: 19 | |
| 1 | MKSNDPIPKD SPLDHTMNLM REGYEFLSHR MERFQTDLFE TRVMGQKVLC IRGAEAVKLF |
| 61 | YDPERFKRHR ATPKRIQKSL FGENAIQTMD DKAHLHRKQL FLSMMKPEDE QELARLTHET |
| 121 | WRRVAEGWKK SRPIVLFDEA KRVLCQVACE WAEVPLKSTE IDRRAEDFHA MVDAFGAVGP |
| 181 | RHWRGRKGRR RTERWIQSII HQVRTGSLQA REGSPLYKVS YHRELNGKLL DERMAAIELI |
| 241 | NVLRPIVAIA TFISFAAIAL QEHPEWQERL KNGSNEEFHM FVQEVRRYYP FAPLIGAKVR |
| 301 | KSFTWKGVRF KKGRLVFLDM YGTNHDPKLW DEPDAFRPER FQERKDSLYD FIPQGGGDPT |
| 361 | KGHRCPGEGI TVEVMKTTMD FLVNDIDYDV PDQDISYSLS RMPTRPESGY IMANIERKYE |
| 421 | HA |
| aryC | |
| Streptomyces parvus | |
| GenBank Accession No.: AFM80022 | |
| >gi\|392601346\|gb\|AFM80022.1\| cytochrome P450 +Streptomyces parvus+ | |
| SEQ ID NO: 20 | |
| 1 | MYLGGRRGTE AVGESREPGV WEVFRYDEAV QVLGDHRTFS SDMNHFIPEE QRQLARAARG |
| 61 | NFVGIDPPDH TQLRGLVSQA FSPRVTAALE PRIGRLAEQL LDDIVAERGD KASCDLVGEF |
| 121 | AGPLSAIVIA ELFGIPESDH TMIAEWAKAL LGSRPAGELS IADEAAMQNT ADLVRRAGEY |
| 181 | LVHHITERRA RPQDDLTSRL ATTEVDGKRL DDEEIVGVIG MFLIAGYLPA SVLTANTVMA |
| 241 | LDEHPAALAE VRSDPALLPG AIEEVLRWRP PLVRDQRLTT RDADLGGRTV PAGSMVCVWL |
| 301 | ASAHRDPFRF ENPDLFDIHR NAGRHLAFGK GIHYCLGAPL ARLEARIAVE TLLRRFERIE |
| 361 | IPRDESVEFH ESIGVLGPVR LPTTLFARR |
| CYP101A1 | |
| Pseudomonas putida | |
| Uniprot Accession No.: P00183 | |
| >sp\|P00183\|CPXA_PSEPU Camphor 5-monooxygenase OS = Pseudomonas putida | |
| GN = camC PE = 1 SV = 2 | |
| SEQ ID NO: 21 | |
| TTETIQSNANLAPLPPHVPEHLVEDFDMYNPSNLSAGVQEAWAVLQESNVPDLVWTRCNGGHWIATRGQLIREAYED | |
| YRHESSECPFIPREAGEAYDFIPTSMDPPEQRQFRALANQVVGMPVVDKLENRIQELACSLIESLRPQGQCNFTEDY | |
| AEPFPIRIFMLLAGLPEEDIPHLKYLTDQMTRPDGSMTFAEAKEALYDYLIPIIEQRRQKPGTDAISIVANGQVNGR | |
| PITSDEAKRMCGLLLVGGLDTVVNFLSFSMEFLAKSPEHRQELIERPERIPAACEELLRRFSLVADGRILTSDYEFH | |
| GVQLKKGDQILLPQMLSGLDERENACPMHVDFSRQKVSHTTFGHGSHLCLGQHLARREIIVTLKEWLTRIPDFSIAP | |
| GAQIQHKSGIVSGVQALPLVWDPATTKAV | |
| Homo sapiens | |
| CYP2D7 | |
| GenBank Accession No.: AA049806 | |
| >gi\|37901459\|gb\|AA049806.1\| cytochrome P450 [Homo sapiens] | |
| SEQ ID NO: 22 | |
| GLEALVPLA MIVAIFLLLV DLMHRHQRWA ARYPPGPLPL PGLGNLLHVD FQNTPYCFDQ | |
| LRRRFGDVFN LQLAWTPVVV LNGLAAVREA MVTRGEDTAD RPPAPIYQVL GFGPRSQGVI | |
| LSRYGPAWRE QRRFSVSTLR NLGLGKKSLE QWVTEEAACL CAAFADQAGR PFRPNGLLDK | |
| AVSNVIASLT CGRRFEYDDP RFLRLLDLAQ EGLKEESGFL REVLNAVPVL PHIPALAGKV | |
| LRFQKAFLTQ LDELLTEHRM TWDPAQPPRD LTEAFLAKKE KAKGSPESSF NDENLRIVVG | |
| NLFLAGMVTT LTTLAWGLLL MILHLDVQRG RRVSPGCSPI VGTHVCPVRV QQEIDDVIGQ | |
| VRRPEMGDQV HMPYTTAVIH EVQRFGDIVP LGVTHMTSRD IEVQGFRIPK GTTLITNLSS | |
| VLKDEAVWEK PFRFHPEHFL DAQGHFVKPE AFLPFSAGRR ACLGEPLARM ELFLFFTSLL | |
| QHFSFSVAAG QPRPSHSRVV SFLVTPSPYE LCAVPR | |
| Rattus norvegicus | |
| CYPC27 | |
| GenBank Accession No.: AAB02287 | |
| >gi\|1374714\|gb\|AAB02287.1\| cytochrome P450 [Rattus norvegicus] | |
| SEQ ID NO: 23 | |
| AVLSRMRLRWALLDTRVMGHGLCPQGARAKAAIPAALRDHESTEGPGTGQDRPRLRSLAELPGPGTLRF | |
| LFQLFLRGYVLHLHELQALNKAKYGPMWTTTEGTRTNVNLASAPLLEQVMRQEGKYPIRDSMEQWKEHRD | |
| HKGLSYGIFITQGQQWYHLRHSLNQRMLKPAEAALYTDALNEVISDFIARLDQVRTESASGDQVPDVAHL | |
| LYHLALEAICYILFEKRVGCLEPSIPEDTATFIRSVGLMEKNSVYVTFLPKWSRPLLPFWKRYMNNWDNI | |
| FSFGEKMIHQKVQEIEAQLQAAGPDGVQVSGYLHFLLTKELLSPQETVGTFPELILAGVDTTSNTLTWAL | |
| YHLSKNPEIQEALHKEVTGVVPFGKVPQNKDFAHMPLLKAVIKETLRLYPVVPTNSRIITEKETEINGFL | |
| FPKNTQFVLCTYVVSRDPSVFPEPESFQPHRWLRKREDDNSGIQHPFGSVPFGYGVRSCLGRRIAELEMQ | |
| LLLSRLIQKYEVVLSPGMGEVKSVSRIVLVPSKKVSLRFLQRQ | |
| CYP2B4 | |
| Oryctolagus cuniculus | |
| GenBank Accession No. AAA65840 | |
| >gi\|164959\|gb\|AAA65840.1\| cytochrome P-450 [Oryctolagus cuniculus] | |
| SEQ ID NO: 24 | |
| MEFSLLLLLAFLAGLLLLLFRGHPKAHGRLPPGPSPLPVLGNLLQMDRKGLLRSFLRLRE | |
| KYGDVFTVYLGSRPVVVLCGTDAIREALVDQAEAFSGRGKIAVVDPIFQGYGVIFANGER | |
| WRALRRFSLATMRDFGMGKRSVEERIQEEARCLVEELRKSKGALLDNTLLFHSITSNIIC | |
| SIVEGKREDYKDPVFLRLLDLFFQSFSLISSFSSQVFELFPGFLKHFPGTHRQIYRNLQE | |
| INTFIGQSVEKHRATLDPSNPRDFIDVYLLRMEKDKSDPSSEFHHQNLILTVLSLFFAGT | |
| ETTSTTLRYGELLMLKYPHVTERVQKEIEQVIGSHRPPALDDRAKMPYTDAVIHEIQRLG | |
| DLIPFGVPHTVTKDTQFRGYVIPKNTEVFPVLSSALHDPRYFETPNTENPGHFLDANGAL | |
| KRNEGFMPFSLGKRICLGEGIARTELFLFFTTILQNFSIASPVPPEDIDLTPRESGVGNV | |
| PPSYQIRFLAR | |
| CYP102A2 | |
| Bacillus subtilis | |
| Uniprot Accession No. 008394 | |
| >sp\|O08394\|CYPD_BACSU Probable bifunctional P-450/NADPH-P450 | |
| reductase 1 OS = Bacillus subtilis (strain 168) GN = cypD | |
| PE = 3 SV = 1 | |
| SEQ ID NO: 25 | |
| MKETSPIPQPKTFGPLGNLPLIDKDKPTLSLIKLAEEQGPIFQIHTPAGTTIVVSGHELV | |
| KEVCDEERFDKSIEGALEKVRAFSGDGLFTSWTHEPNWRKAHNILMPTESQRAMKDYHEK | |
| MVDIAVQLIQKWARLNPNEAVDVPGDMTRLTLDTIGLCGFNYRFNSYYRETPHPFINSMV | |
| RALDEAMHQMQRLDVQDKLMVRTKRQFRHDIQTMESLVDSIIAERRANGDQDEKDLLARM | |
| LNVEDPETGEKLDDENIRFQIITFLIAGHETTSGLLSFATYFLLKHPDKLKKAYEEVDRV | |
| LTDAAPTYKQVLELTYIRMILNESLRLWPTAPAFSLYPKEDTVIGGKFPITTNDRISVLI | |
| PQLHRDRDAWGKDAEEFRPERFEHQDQVPHHAYKPFGNGQRACIGMQFALHEATLVLGMI | |
| LKYFTLIDHENYELDIKQTLTLKPGDFHIRVQSRNQDAIHADVQAVEKAASDEQKEKTEA | |
| KGTSVIGLNNRPLLVLYGSDTGTAEGVARELADTASLHGVRTETAPLNDRIGKLPKEGAV | |
| VIVTSSYNGKPPSNAGQFVQWLQEIKPGELEGVHYAVEGCGDHNWASTYQYVPRFIDEQL | |
| AEKGATRFSARGEGDVSGDFEGQLDEWKKSMWADAIKAFGLELNENADKERSTLSLQFVR | |
| GLGESPLARSYEASHASIAENRELQSADSDRSTRHIEIALPPDVEYQEGDHLGVLPKNSQ | |
| TNVSRILHREGLKGTDQVTLSASGRSAGHLPLGRPVSLHDLLSYSVEVQEAATRAQIREL | |
| AAFTVCPPHRRELEELSAEGVYQEQILKKRISMLDLLEKYEACDMPFERFLELLRPLKPR | |
| YYSISSSPRVNPRQASITVGVVRGPAWSGRGEYRGVASNDLAERQAGDDVVMFIRTPESR | |
| FQLPKDPETPIIMVGPGTGVAPERGELQARDVLKREGKTLGEAHLYFGCRNDRDFIYRDE | |
| LERFEKDGIVTVHTAFSRKEGMPKTYVQHLMADQADTLISILDRGGRLYVCGDGSKMAPD | |
| VEAALQKAYQAVHGTGEQEAQNWLRHLQDTGMYAKDVWAGI | |
| CYP102A3 | |
| Bacillus subtilis | |
| Uniprot Accession No.008336 | |
| >sp\|008336\|CYPE_BACSU Probable bifunctional P-450/NADPH-P450 | |
| reductase 2 OS = Bacillus subtilis (strain 168) GN = cypE | |
| PE = 2 SV = 1 | |
| SEQ ID NO: 26 | |
| MKQASAIPQPKTYGPLKNLPHLEKEQLSQSLWRIADELGPIFREDFPGVSSVFVSGHNLV | |
| AEVCDESREDKNLGKGLQKVREFGGDGLFTSWTHEPNWQKAHRILLPSFSQKAMKGYHSM | |
| MLDIATQLIQKWSRLNPNEEIDVADDMTRLTLDTIGLCGFNYRFNSFYRDSQHPFITSML | |
| RALKEAMNQSKRLGLQDKMMVKTKLQFQKDIEVMNSLVDRMIAERKANPDDNIKDLLSLM | |
| LYAKDPVTGETLDDENIRYQIITFLIAGHETTSGLLSFAIYCLLTHPEKLKKAQEEADRV | |
| LTDDTPEYKQIQQLKYTRMVLNETLRLYPTAPAFSLYAKEDTVLGGEYPISKGQPVTVLI | |
| PKLHRDQNAWGPDAEDFRPERFEDPSSIPHHAYKPFGNGQRACIGMQFALQEATMVLGLV | |
| LKHFELINHTGYELKIKEALTIKPDDFKITVKPRKTAAINVQRKEQADIKAETKPKETKP | |
| KHGTPLLVLYGSNLGTAEGIAGELAAQGRQMGETAETAPLDDYIGKLPEEGAVVIVTASY | |
| NGSPPDNAAGFVEWLKELEEGQLKGVSYAVEGCGNRSWASTYQRIPRLIDDMMKAKGASR | |
| LTE IGEGDAADDFESHRESWENRFWKETMDAFDINEIAQKEDRPSLSIAFLSEATETPVA | |
| KAYGAFEGVVLENRELQTADSTRSTRHIELEIPAGKTYKEGDHIGIMPKNSRELVQRVLS | |
| REGLQSNHVIKVSGSAHMSHLPMDRPIKVADLLSSYVELQEPASRLQLRELASYTVCPPH | |
| QKELEQLVLDDGIYKEQVLAKRLTMLDFLEDYPACEMPFERFLALLPSLKPRYYSISSSP | |
| KVHANIVSMTVGVVKASAWSGRGEYRGVASNYLAELNTGDAAACFIRTPQSGFQMPDEPE | |
| TPMIMVGPGTGIAPERGFIQARSVLKKEGSTLGEALLYFGCRRPDHDDLYREELDQAEQE | |
| GLVTIRRCYSRVENESKGYVQHLLKQDSQKLMTLIEKGAHIYVCGDGSQMAPDVEKTLRW | |
| AYETEKGASQEESADWLQKLQDQKRYIKDVWTGN | |
| CYP102A1 | |
| B. megaterium DSM 32 | |
| Uniprot Accession No. P14779 | |
| >sp\|P14779\|CPXB_BACME Bifunctional P-450/NADPH-P450 reductase OS+32Bacillus | |
| megaterium GN = cyp102A1 PE = 1 SV = 2 | |
| SEQ ID NO: 27 | |
| 1 | MTIKEMPQPK TFGELKNLPL LNTDKPVQAL MKIADELGEI FKFEAPGRVT RYLSSQRLIK |
| 61 | EACDESRFDK NLSQALKFVR DFAGDGLFTS WTHEKNWKKA HNILLPSFSQ QAMKGYHAMM |
| 121 | VDIAVQLVQK WERLNADEHI EVPEDMTRLT LDTIGLCGFN YRFNSFYRDQ PHPFITSMVR |
| 181 | ALDEAMNKLQ RANPDDPAYD ENKRQFQEDI KVMNDLVDKI IADRKASGEQ SDDLLTHMLN |
| 241 | GKDPETGEPL DDENIRYQII TFLIAGHETT SGLLSFALYF LVKNPHVLQK AAEEAARVLV |
| 301 | DPVPSYKQVK QLKYVGMVLN EALRLWPTAP AFSLYAKEDT VLGGEYPLEK GDELMVLIPQ |
| 361 | LHRDKTIWGD DVEEFRPERF ENPSAIPQHA FKPFGNGQRA CIGQQFALHE ATLVLGMMLK |
| 421 | HFDFEDHTNY ELDIKETLTL KPEGFVVKAK SKKIPLGGIP SPSTEQSAKK VRKKAENAHN |
| 481 | TPLLVLYGSN MGTAEGTARD LADIAMSKGF APQVATLDSH AGNLPREGAV LIVTASYNGH |
| 541 | PPDNAKQFVD WLDQASADEV KGVRYSVFGC GDKNWATTYQ KVPAFIDETL AAKGAENIAD |
| 601 | RGEADASDDF EGTYEEWREH MWSDVAAYFN LDIENSEDNK STLSLQFVDS AADMPLAKMH |
| 661 | GAFSTNVVAS KELQQPGSAR STRHLEIELP KEASYQEGDH LGVIPRNYEG IVNRVTARFG |
| 721 | LDASQQIRLE AEEEKLAHLP LAKTVSVEEL LQYVELQDPV TRTQLRAMAA KTVCPPHKVE |
| 781 | LEALLEKQAY KEQVLAKRLT MLELLEKYPA CEMKFSEFIA LLPSIRPRYY SISSSPRVDE |
| 841 | KQASITVSVV SGEAWSGYGE YKGIASNYLA ELQEGDTITC FISTPQSEFT LPKDPETPLI |
| 901 | MVGPGTGVAP FRGFVQARKQ LKEQGQSLGE AHLYFGCRSP HEDYLYQEEL ENAQSEGIIT |
| 961 | LHTAFSRMPN QPKTYVQHVM EQDGKKLIEL LDQGAHFYIC GDGSQMAPAV EATLMKSYAD |
| 1021 | VHQVSEADAR LWLQQLEEKG RYAKDVWAG |
| CYP102A5 | |
| B. cereus ATCC14579 | |
| GenBank Accession No. AAP10153 | |
| >gi\|29896875\|gb\|AAP10153.1\| NADPH-cytochrome P450 reductase | |
| [Bacillus cereus ATCC 14579] | |
| SEQ ID NO: 28 | |
| 1 | MEKKVSAIPQ PKTYGPLGNL PLIDKDKPTL SFIKIAEEYG PIFQIQTLSD TIIVVSGHEL |
| 61 | VAEVCDETRF DKSIEGALAK VRAFAGDGLF TSETHEPNWK KAHNILMPTF SQRAMKDYHA |
| 121 | MMVDIAVQLV QKWARLNPNE NVDVPEDMTR LTLDTIGLCG FNYRFNSFYR ETPHPFITSM |
| 181 | TRALDEAMHQ LQRLDIEDKL MWRTKRQFQH DIQSMFSLVD NIIAERKSSG DQEENDLLSR |
| 241 | MLNVPDPETG EKLDDENIRF QIITFLIAGH ETTSGLLSFA IYFLLKNPDK LKKAYEEVDR |
| 301 | VLTDPTPTYQ QVMKLKYMRM ILNESLRLWP TAPAFSLYAK EDTVIGGKYP IKKGEDRISV |
| 361 | LIPQLHRDKD AWGDNVEEFQ PERFEELDKV PHHAYKPFGN GQRACIGMQF ALHEATLVMG |
| 421 | MLLQHFELID YQNYQLDVKQ TLTLKPGDFK IRILPRKQTI SHPTVLAPTE DKLKNDEIKQ |
| 481 | HVQKTPSIIG ADNLSLLVLY GSDTGVAEGI ARELADTASL EGVQTEVVAL NDRIGSLPKE |
| 541 | GAVLIVTSSY NGKPPSNAGQ FVQWLEELKP DELKGVQYAV FGCGDHNWAS TYQRIPRYID |
| 601 | EQMAQKGATR FSKRGEADAS GDFEEQLEQW KQNMWSDAMK AFGLELNKNM EKERSTLSLQ |
| 661 | FVSRLGGSPL ARTYEAVYAS ILENRELQSS SSDRSTRHIE VSLPEGATYK EGDHLGVLPV |
| 721 | NSEKNINRIL KRFGLNGKDQ VILSASGRSI NHIPLDSPVS LLALLSYSVE VQEAATRAQI |
| 781 | REMVTFTACP PHKKELEALL EEGVYHEQIL KKRISMLDLL EKYEACEIRF ERFLELLPAL |
| 841 | KPRYYSISSS PLVAHNRLSI TVGVVNAPAW SGEGTYEGVA SNYLAQRHNK DEIICFIRTP |
| 901 | QSNFELPKDP ETPIIMVGPG TGIAPFRGFL QARRVQKQKG MNLGQAHLYF GCRHPEKDYL |
| 961 | YRTELENDER DGLISLHTAF SRLEGHPKTY VQHLIKQDRI NLISLLDNGA HLYICGDGSK |
| 1021 | MAPDVEDTLC QAYQEIHEVS EQEARNWLDR VQDEGRYGKD VWAGI |
| CYP102A7 | |
| B. licheniformis ATTC1458 | |
| GenBank Accession No. YP 079990 | |
| >gi\|52081199\|ref\|YP_079990.11 cytochrome P450/NADPH-ferrihemoprotein | |
| reductase [Bacillus licheniformis DSM 13 = ATCC 14580] | |
| SEQ ID NO: 29 | |
| 1 | MNKLDGIPIP KTYGPLGNLP LLDKNRVSQS LWKIADEMGP IFQFKFADAI GVFVSSHELV |
| 61 | KEVSEESRFD KNMGKGLLKV REFSGDGLFT SWTEEPNWRK AHNILLPSFS QKAMKGYHPM |
| 121 | MQDIAVQLIQ KWSRLNQDES IDVPDDMTRL TLDTIGLCGF NYRFNSFYRE GQHPFIESMV |
| 181 | RGLSEAMRQT KRFPLQDKLM IQTKRRFNSD VESMFSLVDR IIADRKQAES ESGNDLLSLM |
| 241 | LHAKDPETGE KLDDENIRYQ IITFLIAGHE TTSGLLSFAI YLLLKHPDKL KKAYEEADRV |
| 301 | LTDPVPSYKQ VQQLKYIRMI LNESIRLWPT APAFSLYAKE ETVIGGKYLI PKGQSVTVLI |
| 361 | PKLHRDQSVW GEDAEAFRPE RFEQMDSIPA HAYKPFGNGQ RACIGMQFAL HEATLVLGMI |
| 421 | LQYFDLEDHA NYQLKIKESL TLKPDGFTIR VRPRKKEAMT AMPGAQPEEN GRQEERPSAP |
| 481 | AAENTHGTPL LVLYGSNLGT AEEIAKELAE EAREQGFHSR TAELDQYAGA IPAEGAVIIV |
| 541 | TASYNGNPPD CAKEFVNWLE HDQTDDLRGV KYAVFGCGNR SWASTYQRIP RLIDSVLEKK |
| 601 | GAQRLHKLGE GDAGDDFEGQ FESWKYDLWP LLRTEFSLAE PEPNQTETDR QALSVEFVNA |
| 661 | PAASPLAKAY QVFTAKISAN RELQCEKSGR STRHIEISLP EGAAYQEGDH LGVLPQNSEV |
| 721 | LIGRVFQRFG LNGNEQILIS GRNQASHLPL ERPVHVKDLF QHCVELQEPA TRAQIRELAA |
| 781 | HTVCPPHQRE LEDLLKDDVY KDQVLNKRLT MLDLLEQYPA CELPFARFLA LLPPLKPRYY |
| 841 | SISSSPQLNP RQTSITVSVV SGPALSGRGH YKGVASNYLA GLEPGDAISC FIREPQSGFR |
| 901 | LPEDPETPVI MVGPGTGIAP YRGFLQARRI QRDAGVKLGE AHLYFGCRRP NEDFLYRDEL |
| 961 | EQAEKDGIVH LHTAFSRLEG RPKTYVQDLL REDAALLIHL LNEGGRLYVC GDGSRMAPAV |
| 1021 | EQALCEAYRI VQGASREESQ SWLSALLEEG RYAKDVWDGG VSQHNVKADC IART |
| CYPX | |
| B. thuringiensis serovar konkukian | |
| str.97-27 | |
| GenBank Accession No. YP 037304 | |
| >gi\|49480099\|ref\|YP_037304.11 NADPH-cytochrome P450 reductase | |
| [Bacillus thuringiensis serovar konkukian str. 97-27] | |
| SEQ ID NO: 30 | |
| 1 | MDKKVSAIPQ PKTYGPLGNL PLIDKDKPTL SFIKLAEEYG PIFQIQTLSD TIIVVSGHEL |
| 61 | VAEVCDETRF DKSIEGALAK VRAFAGDGLF TSETDEPNWK KAHNILMPTF SQRAMKDYHA |
| 121 | MMVDIAVQLV QKWARLNPNE NVDVPEDMTR LTLDTIGLCG FNYRFNSFYR ETPHPFITSM |
| 181 | TRALDEAMHQ LQRLDIEDKL MWRTKRQFQH DIQSMFSLVD NIIAERKSSE NQEENDLLSR |
| 241 | MLNVQDPETG EKLDDENIRF QIITFLIAGH ETTSGLLSFA IYFLLKNPDK LKKAYEEVDR |
| 301 | VLTDSTPTYQ QVMKLKYIRM ILNESLRLWP TAPAFSLYAK EDTVIGGKYP IKKGEDRISV |
| 361 | LIPQLHRDKD AWGDDVEEFQ PERFEELDKV PHHAYKPFGN GQRACIGMQF ALHEATLVMG |
| 421 | MLLQHFEFID YEDYQLDVKQ TLTLKPGDFK IRIVPRNQTI SHTTVLAPTE EKLKKHEIKK |
| 481 | QVQKTPSIIG ADNLSLLVLY GSDTGVAEGI ARELADTASL EGVQTEVVAL NDRIGSLPKE |
| 541 | GAVLIVTSSY NGKPPSNAGQ FVQWLEELKP DELKGVQYAV FGCGDHNWAS TYQRIPRYID |
| 601 | EQMAQKGATR FSTRGEADAS GDFEEQLEQW KQSMWSDAMK AFGLELNKNM EKERSTLSLQ |
| 661 | FVSRLGGSPL ARTYEAVYAS ILENRELQSS SSERSTRHIE ISLPEGATYK EGDHLGVLPI |
| 721 | NNEKNVNRIL KRFGLNGKDQ VILSASGRSV NHIPLDSPVR LYDLLSYSVE VQEAATRAQI |
| 781 | REMVTFTACP PHKKELESLL EDGVYQEQIL KKRISMLDLL EKYEACEIRF ERFLELLPAL |
| 841 | KPRYYSISSS PLVAQDRLSI TVGVVNAPAW SGEGTYEGVA SNYLAQRHNK DEIICFIRTP |
| 901 | QSNFQLPENP ETPIIMVGPG TGIAPFRGFL QARRVQKQKG MKVGEAHLYF GCRHPEKDYL |
| 961 | YRTELENDER DGLISLHTAF SRLEGHPKTY VQHVIKEDRI HLISLLDNGA HLYICGDGSK |
| 1021 | MAPDVEDTLC QAYQEIHEVS EQEARNWLDR LQEEGRYGKD VWAGI |
| CYP102E1 | |
| R. metallidurans CH34 | |
| GenBank Accession No. YP 585608 | |
| >gi\|94312398\|ref\|YP_585608.1\| putative bifunctional P-450:NADPH-P450 | |
| reductase 2 [Cupriavidus metallidurans CH34] | |
| SEQ ID NO: 31 | |
| 1 | MSTATPAAAL EPIPRDPGWP IFGNLFQITP GEVGQHLLAR SRHHDGIFEL DFAGKRVPFV |
| 61 | SSVALASELC DATRFRKIIG PPLSYLRDMA GDGLFTAHSD EPNWGCAHRI LMPAFSQRAM |
| 121 | KAYFDVMLRV ANRLVDKWDR QGPDADIAVA DDMTRLTLDT IALAGFGYDF ASFASDELDP |
| 181 | FVMAMVGALG EAMQKLTRLP IQDRFMGRAH RQAAEDIAYM RNLVDDVIRQ RRVSPTSGMD |
| 241 | LLNLMLEARD PETDRRLDDA NIRNQVITFL IAGHETTSGL LTFALYELLR NPGVLAQAYA |
| 301 | EVDTVLPGDA LPVYADLARM PVLDRVLKET LRLWPTAPAF AVAPFDDVVL GGRYRLRKDR |
| 361 | RISVVLTALH RDPKVWANPE RFDIDRFLPE NEAKLPAHAY MPFGQGERAC IGRQFALTEA |
| 421 | KLALALMLRN FAFQDPHDYQ FRLKETLTIK PDQFVLRVRR RRPHERFVTR QASQAVADAA |
| 481 | QTDVRGHGQA MTVLCASSLG TARELAEQIH AGAIAAGFDA KLADLDDAVG VLPTSGLVVV |
| 541 | VAATYNGRAP DSARKFEAML DADDASGYRA NGMRLALLGC GNSQWATYQA FPRRVFDFFI |
| 601 | TAGAVPLLPR GEADGNGDFD QAAERWLAQL WQALQADGAG TGGLGVDVQV RSMAAIRAET |
| 661 | LPAGTQAFTV LSNDELVGDP SGLWDFSIEA PRTSTRDIRL QLPPGITYRT GDHIAVWPQN |
| 721 | DAQLVSELCE RLDLDPDAQA TISAPHGMGR GLPIDQALPV RQLLTHFIEL QDVVSRQTLR |
| 781 | ALAQATRCPF TKQSIEQLAS DDAEHGYATK VVARRLGILD VLVEHPAIAL TLQELLACTV |
| 841 | PMRPRLYSIA SSPLVSPDVA TLLVGTVCAP ALSGRGQFRG VASTWLQHLP PGARVSASIR |
| 901 | TPNPPFAPDP DPAAPMLLIG PGTGIAPFRG FLEERALRKM AGNAVTPAQL YFGCRHPQHD |
| 961 | WLYREDIERW AGQGVVEVHP AYSVVPDAPR YVQDLLWQRR EQVWAQVRDG ATIYVCGDGR |
| 1021 | RMAPAVRQTL IEIGMAQGGM TDKAASDWFG GLVAQGRYRQ DVFN |
| CYP505X | |
| A. fumigatus Af293 | |
| GenBank Accession No. EAL92660 | |
| >gi\|66852335\|gb\|EAL92660.1\| P450 family fatty acid hydroxylase, putative | |
| [Aspergillus fumigatus Af293] | |
| SEQ ID NO: 32 | |
| 1 | MSESKTVPIP GPRGVPLLGN IYDIEQEVPL RSINLMADQY GPIYRLTTFG WSRVFVSTHE |
| 61 | LVDEVCDEER FTKVVTAGLN QIRNGVHDGL FTANFPGEEN WAIAHRVLVP AFGPLSIRGM |
| 121 | FDEMYDIATQ LVMKWARHGP TVPIMVTDDF TRLTLDTIAL CAMGTRFNSF YHEEMHPFVE |
| 181 | AMVGLLQGSG DRARRPALLN NLPTSENSKY WDDIAFLRNL AQELVEARRK NPEDKKDLLN |
| 241 | ALILGRDPKT GKGLTDESII DNMITFLIAG HETTSGLLSF LFYYLLKTPN AYKKAQEEVD |
| 301 | SVVGRRKITV EDMSRLPYLN AVMRETLRLR STAPLIAVHA HPEKNKEDPV TLGGGKYVLN |
| 361 | KDEPIVIILD KLHRDPQVYG PDAEEFKPER MLDENFEKLP KNAWKPFGNG MRACIGRPFA |
| 421 | WQEALLVVAI LLQNFNFQMD DPSYNLHIKQ TLTIKPKDFH MRATLRHGLD ATKLGIALSG |
| 481 | SADRAPPESS GAASRVRKQA TPPAGQLKPM HIFFGSNTGT CETFARRLAD DAVGYGFAAD |
| 541 | VQSLDSAMQN VPKDEPVVFI TASYEGQPPD NAAHFFEWLS ALKENELEGV NYAVFGCGHH |
| 601 | DWQATFHRIP KAVNQLVAEH GGNRLCDLGL ADAANSDMFT DFDSWGESTF WPAITSKFGG |
| 661 | GKSDEPKPSS SLQVEVSTGM RASTLGLQLQ EGLVIDNQLL SAPDVPAKRM IRFKLPSDMS |
| 721 | YRCGDYLAVL PVNPTSVVRR AIRRFDLPWD AMLTIRKPSQ APKGSTSIPL DTPISAFELL |
| 781 | STYVELSQPA SKRDLTALAD AAITDADAQA ELRYLASSPT RFTEEIVKKR MSPLDLLIRY |
| 841 | PSIKLPVGDF LAMLPPMRVR QYSISSSPLA DPSECSITFS VLNAPALAAA SLPPAERAEA |
| 901 | EQYMGVASTY LSELKPGERA HIAVRPSHSG FKPPMDLKAP MIMACAGSGL APFRGFIMDR |
| 961 | AEKIRGRRSS VGADGQLPEV EQPAKAILYV GCRTKGKDDI HATELAEWAQ LGAVDVRWAY |
| 1021 | SRPEDGSKGR HVQDLMLEDR EELVSLFDQG ARIYVCGSTG VGNGVRQACK DIYLERRRQL |
| 1081 | RQAARERGEE VPAEEDEDAA AEQFLDNLRT KERYATDVFT |
| CYP505A8 | |
| A. nidulans FGSC A4 | |
| GenBank Accession No. EAA58234 | |
| >gi\|40739044\|gb\|EAA58234.1\| hypothetical protein AN6835.2 | |
| [Aspergillus nidulans FGSC A4] | |
| SEQ ID NO: 33 | |
| 1 | MAEIPEPKGL PLIGNIGTID QEFPLGSMVA LAEEHGEIYR LRFPGRTVVV VSTHALVNET |
| 61 | CDEKRFRKSV NSALAHVREG VHDGLFTAKM GEVNWEIAHR VLMPAFGPLS IRGMFDEMHD |
| 121 | IASQLALKWA RYGPDCPIMV TDDFTRLTLD TLALCSMGYR FNSYYSPVLH PFIEAMGDFL |
| 181 | TEAGEKPRRP PLPAVFFRNR DQKFQDDIAV LRDTAQGVLQ ARKEGKSDRN DLLSAMLRGV |
| 241 | DSQTGQKMTD ESIMDNLITF LIAGHETTSG LLSFVFYQLL KHPETYRTAQ QEVDNVVGQG |
| 301 | VIEVSHLSKL PYINSVLRET LRLNATIPLF TVEAFEDTLL AGKYPVKAGE TIVNLLAKSH |
| 361 | LDPEVYGEDA LEFKPERMSD ELFNARLKQF PSAWKPFGNG MRACIGRPFA WQEALLVMAM |
| 421 | LLQNFDFSLA DPNYDLKFKQ TLTIKPKDMF MKARLRHGLT PTTLERRLAG LAVESATQDK |
| 481 | IVTNPADNSV TGTRLTILYG SNSGTCETLA RRIAADAPSK GFHVMRFDGL DSGRSALPTD |
| 541 | HPVVIVTSSY EGQPPENAKQ FVSWLEELEQ QNESLQLKGV DFAVFGCFKE WAQTFHRIPK |
| 601 | LVDSLLEKLG GSRLTDLGLA DVSTDELFST FETWADDVLW PRLVAQYGAD GKTQAHGSSA |
| 661 | GHEAASNAAV EVTVSNSRTQ ALRQDVGQAM VVETRLLTAE SEKERRKKHL EIRLPDGVSY |
| 721 | TAGDYLAVLP INPPETVRRA MRQFKLSWDA QITIAPSGPT TALPTDGPIA ANDIFSTYVE |
| 781 | LSQPATRKDL RIMADATTDP DVQKILRTYA NETYTAEILT KSISVLDILE QHPAIDLPLG |
| 841 | TFLLMLPSMR MRQYSISSSP LLTPTTATIT ISVLDAPSRS RSNGSRHLGV ATSYLDSLSV |
| 901 | GDHLQVTVRK NPSSGFRLPS EPETTPMICI AAGSGIAPFR AFLQERAVMM EQDKDRKLAP |
| 961 | ALLFFGCRAP GIDDLYREQL EEWQARGVVD ARWAFSRQSD DTKGCRHVDD RILADREDVV |
| 1021 | KLWRDGARVY VCGSGALAQS VRSAMVTVLR DEMETTGDGS DNGKAEKWFD EQRNVRYVMD |
| 1081 | VFD |
| CYP505A3 | |
| A. oryzae ATCC42149 | |
| Uniprot Accession No. Q2U4F1 | |
| >gi\|121928062\|sp\|Q2U4F1\|Q2U4F1_ASPOR Cytochrome P450 | |
| SEQ ID NO: 34 | |
| 1 | MRQNDNEKQI CPIPGPQGLP FLGNILDIDL DNGTMSTLKI AKTYYPIFKF TFAGETSIVI |
| 61 | NSVALLSELC DETRFHKHVS FGLELLRSGT HDGLFTAYDH EKNWELAHRL LVPAFGPLRI |
| 121 | REMFPQMHDI AQQLCLKWQR YGPRRPLNLV DDFTRTTLDT IALCAMGYRF NSFYSEGDFH |
| 181 | PFIKSMVRFL KEAETQATLP SFISNLRVRA KRRTQLDIDL MRTVCREIVT ERRQTNLDHK |
| 241 | NDLLDTMLTS RDSLSGDALS DESIIDNILT FLVAGHETTS GLLSFAVYYL LTTPDAMAKA |
| 301 | AHEVDDVVGD QELTIEHLSM LKYLNAILRE TLRLMPTAPG FSVTPYKPEI IGGKYEVKPG |
| 361 | DSLDVFLAAV HRDPAVYGSD ADEFRPERMS DEHFQKLPAN SWKPFGNGKR SCIGRAFAWQ |
| 421 | EALMILALIL QSFSLNLVDR GYTLKLKESL TIKPDNLWAY ATPRPGRNVL HTRLALQTNS |
| 481 | THPEGLMSLK HETVESQPAT ILYGSNSGTC EALAHRLAIE MSSKGRFVCK VQPMDAIEHR |
| 541 | RLPRGQPVII ITGSYDGRPP ENARHFVKWL QSLKGNDLEG IQYAVFGCGL PGHHDWSTTF |
| 601 | YKIPTLIDTI MAEHGGARLA PRGSADTAED DPFAELESWS ERSVWPGLEA AFDLVRHNSS |
| 661 | DGTGKSTRIT IRSPYTLRAA HETAVVHQVR VLTSAETTKK VHVELALPDT INYRPGDHLA |
| 721 | ILPLNSRQSV QRVLSLFQIG SDTILYMTSS SATSLPTDTP ISAHDLLSGY VELNQVATPT |
| 781 | SLRSLAAKAT DEKTAEYLEA LATDRYTTEV RGNHLSLLDI LESYSVPSIE IQHYIQMLPL |
| 841 | LRPRQYTISS SPRLNRGQAS LTVSVMERAD VGGPRNCAGV ASNYLASCTP GSILRVSLRQ |
| 901 | ANPDFRLPDE SCSHPIIMVA AGSGIAPFRA FVQERSVRQK EGIILPPAFL FFGCRRADLD |
| 961 | DLYREELDAF EEQGVVTLFR AFSRAQSESH GCKYVQDLLW MERVRVKTLW GQDAKVFVCG |
| 1021 | SVRMNEGVKA IISKIVSPTP TEELARRYIA ETFI |
| CYPX | |
| A. oryzae ATCC42149 | |
| Uniprot Accession No. Q2UNA2 | |
| >gi\|121938553\|sp\|Q2UNA2\|Q2UNA2_ASPOR Cytochrome P450 | |
| SEQ ID NO: 35 | |
| 1 | MSTPKAEPVP IPGPRGVPLM GNILDIESEI PLRSLEMMAD TYGPIYRLTT FGFSRCMISS |
| 61 | HELAAEVFDE ERFTKKIMAG LSELRHGIHD GLFTAHMGEE NWEIAHRVLM PAFGPLNIQN |
| 121 | MFDEMHDIAT QLVMKWARQG PKQKIMVTDD FTRLTLDTIA LCAMGTRFNS FYSEEMHPFV |
| 181 | DAMVGMLKTA GDRSRRPGLV NNLPTTENNK YWEDIDYLRN LCKELVDTRK KNPTDKKDLL |
| 241 | NALINGRDPK TGKGMSYDSI IDNMITFLIA GHETTSGSLS FAFYNMLKNP QAYQKAQEEV |
| 301 | DRVIGRRRIT VEDLQKLPYI TAVMRETLRL TPTAPAIAVG PHPTKNHEDP VTLGNGKYVL |
| 361 | GKDEPCALLL GKIQRDPKVY GPDAEEFKPE RMLDEHFNKL PKHAWKPFGN GMRACIGRPF |
| 421 | AWQEALLVIA MLLQNFNFQM DDPSYNIQLK QTLTIKPNHF YMRAALREGL DAVHLGSALS |
| 481 | ASSSEHADHA AGHGKAGAAK KGADLKPMHV YYGSNTGTCE AFARRLADDA TSYGYSAEVE |
| 541 | SLDSAKDSIP KNGPVVFITA SYEGQPPDNA AHFFEWLSAL KGDKPLDGVN YAVFGCGHHD |
| 601 | WQTTFYRIPK EVNRLVGENG ANRLCEIGLA DTANADIVTD FDTWGETSFW PAVAAKFGSN |
| 661 | TQGSQKSSTF RVEVSSGHRA TTLGLQLQEG LVVENTLLTQ AGVPAKRTIR FKLPTDTQYK |
| 721 | CGDYLAILPV NPSTVVRKVM SRFDLPWDAV LRIEKASPSS SKHISIPMDT QVSAYDLFAT |
| 781 | YVELSQPASK RDLAVLADAA AVDPETQAEL QAIASDPARF AEISQKRISV LDLLLQYPSI |
| 841 | NLAIGDFVAM LPPMRVRQYS ISSSPLVDPT ECSITFSVLK APSLAALTKE DEYLGVASTY |
| 901 | LSELRSGERV QLSVRPSHTG FKPPTELSTP MIMACAGSGL APFRGFVMDR AEKIRGRRSS |
| 961 | GSMPEQPAKA ILYAGCRTQG KDDIHADELA EWEKIGAVEV RRAYSRPSDG SKGTHVQDLM |
| 1021 | MEDKKELIDL FESGARIYVC GTPGVGNAVR DSIKSMFLER REEIRRIAKE KGEPVSDDDE |
| 1081 | ETAFEKFLDD MKTKERYTTD IFA |
| CYP505A1 | |
| F. oxysporum | |
| Uniprot Accession No. Q9Y8G7 | |
| >gi\|22653677\|sp\|Q9Y8G7.1\|C505_FUSOX RecName: Full = Bifunctional | |
| P-450:NADPH-P450 reductase; AltName: Full = Cytochrome P450foxy; | |
| AltName: Full = Fatty acid omega-hydroxylase; Includes: RecName: | |
| Full = Cytochrome P450 505; Includes: | |
| RecName: Full = NADPH--cytochrome P450 reductase | |
| SEQ ID NO: 36 | |
| 1 | maesvpipep pgyplignlg eftsnplsdl nrladtygpi frlrlgakap ifvssnslin |
| 61 | evcdekrfkk tlksvlsqvr egvhdglfta fedepnwgka hrilvpafgp lsirgmfpem |
| 121 | hdiatqlcmk farhgprtpi dtsdnftrla ldtlalcamd frfysyykee lhpfieamgd |
| 181 | fltesgnrnr rppfapnfly raanekfygd ialmksvade vvaarkasps drkdllaaml |
| 241 | ngvdpqtgek lsdenitnql itfliaghet tsgtlsfamy qllknpeays kvqkevdevv |
| 301 | grgpvlvehl tklpyisavl retlrinspi tafgleaidd tflggkylvk kgeivtalls |
| 361 | rghvdpvvyg ndadkfiper mlddefarin keypncwkpf gngkracigr pfawqeslla |
| 421 | mvvlfqnfnf tmtdpnyale ikqtltikpd hfyinatlrh gmtptelehv lagngatsss |
| 481 | thnikaaanl dakagsgkpm aifygsnsgt cealanrlas dapshgfsat tvgpldqakq |
| 541 | nlpedrpvvi vtasyeggpp snaahfikwm edldgndmek vsyavfacgh hdwvetfhri |
| 601 | pklvdstlek rggtrlvpmg sadaatsdmf sdfeawediv lwpglkekyk isdeesggqk |
| 661 | gllvevstpr ktslrqdvee alvvaektlt ksgpakkhie iqlpsamtyk agdylailpl |
| 721 | npkstvarvf rrfslawdsf lkiqsegptt lptnvaisaf dvfsayvels qpatkrnila |
| 781 | laeatedkdt iqelerlagd ayqaeispkr vsvldllekf pavalpissy lamlppmrvr |
| 841 | gysissspfa dpskltltys lldapslsgq grhvgvatnf lshltagdkl hvsvrassea |
| 901 | fhlpsdaekt piicvaagtg laplrgfiqe raamlaagrt lapallffgc rnpeiddlya |
| 961 | eeferwekmg avdvrraysr atdksegcky vqdrvyhdra dvfkvwdqga kvficgsrei |
| 1021 | gkavedvcvr laiekaqqng rdvteemara wfersrnerf atdvfd |
| CYPX | |
| G. moniliformis | |
| GenBank Accession No. AAG27132 | |
| >gi\|11035011\|gb\|AAG27132.11 Fum6p [Fusarium verticillioides] | |
| SEQ ID NO: 37 | |
| 1 | MSATALFTRR SVSTSNPELR PIPGPKPLPL LGNLFDFDFD NLTKSLGELG KIHGPIYSIT |
| 61 | FGASTEIMVT SREIAQELCD ETRFCKLPGG ALDVMKAVVG DGLFTAETSN PKWAIAHRII |
| 121 | TPLFGAMRIR GMFDDMKDIC EQMCLRWARF GPDEPLNVCD NMTKLTLDTI ALCTIDYRFN |
| 181 | SFYRENGAAH PFAEAVVDVM TESFDQSNLP DFVNNYVRFR AMAKFKRQAA ELRRQTEELI |
| 241 | AARRQNPVDR DDLLNAMLSA KDPKTGEGLS PESIVDNLLT FLIAGHETTS SLLSFCFYYL |
| 301 | LENPHVLRRV QQEVDTVVGS DTITVDHLSS MPYLEAVLRE TLRLRDPGPG FYVKPLKDEV |
| 361 | VAGKYAVNKD QPLFIVFDSV HRDQSTYGAD ADEFRPERML KDGFDKLPPC AWKPFGNGVR |
| 421 | ACVGRPFAMQ QAILAVAMVL HKFDLVKDES YTLKYHVTMT VRPVGFTMKV RLRQGQRATD |
| 481 | LAMGLHRGHS QEASAAASPS RASLKRLSSD VNGDDTDHKS QIAVLYASNS GSCEALAYRL |
| 541 | AAEATERGFG IRAVDVVNNA IDRIPVGSPV ILITASYNGE PADDAQEFVP WLKSLESGRL |
| 601 | NGVKFAVFGN GHRDWANTLF AVPRLIDSEL ARCGAERVSL MGVSDTCDSS DPFSDFERWI |
| 661 | DEKLFPELET PHGPGGVKNG DRAVPRQELQ VSLGQPPRIT MRKGYVRAIV TEARSLSSPG |
| 721 | VPEKRHLELL LPKDFNYKAG DHVYILPRNS PRDVVRALSY FGLGEDTLIT IRNTARKLSL |
| 781 | GLPLDTPITA TDLLGAYVEL GRTASLKNLW TLVDAAGHGS RAALLSLTEP ERFRAEVQDR |
| 841 | HVSILDLLER FPDIDLSLSC FLPMLAQIRP RAYSFSSAPD WKPGHATLTY TVVDFATPAT |
| 901 | QGINGSSKSK AVGDGTAVVQ RQGLASSYLS SLGPGTSLYV SLHRASPYFC LQKSTSLPVI |
| 961 | MVGAGTGLAP FRAFLQERRM AAEGAKQRFG PALLFFGCRG PRLDSLYSVE LEAYETIGLV |
| 1021 | QVRRAYSRDP SAQDAQGCKY VTDRLGKCRD EVARLWMDGA QVLVCGGKKM ANDVLEVLGP |
| 1081 | MLLEIDQKRG ETTAKTVVEW RARLDKSRYV EEVYV |
| CYP505A7 | |
| G. zeae PH1 | |
| GenBank Accession No. EAA67736 | |
| >gi\|42544893\|gb\|EAA67736.1\| C505_FUSOX Bifunctional P-450:NADPH-P450 | |
| reductase (Fatty acid omega-hydroxylase) (P450foxy) | |
| [Gibberella zeae PH-1] | |
| SEQ ID NO: 38 | |
| 1 | MAESVPIPEP PGYPLIGNLG EFKTNPLNDL NRLADTYGPI FRLHLGSKTP TFVSSNAFIN |
| 61 | EVCDEKRFKK TLKSVLSVVR EGVHDGLFTA FEDEPNWGKA HRILIPAFGP LSIRNMFPEM |
| 121 | HEIANQLCMK LARHGPHTPV DASDNFTRLA LDTLALCAMD FRFNSYYKEE LHPFIEAMGD |
| 181 | FLLESGNRNR RPAFAPNFLY RAANDKFYAD IALMKSVADE VVATRKQNPT DRKDLLAAML |
| 241 | EGVDPQTGEK LSDDNITNQL ITFLIAGHET TSGTLSFAMY HLLKNPEAYN KLQKEIDEVI |
| 301 | GRDPVTVEHL TKLPYLSAVL RETLRISSPI TGFGVEAIED TFLGGKYLIK KGETVLSVLS |
| 361 | RGHVDPVVYG PDAEKFVPER MLDDEFARLN KEFPNCWKPF GNGKRACIGR PFAWQESLLA |
| 421 | MALLFQNFNF TQTDPNYELQ IKQNLTIKPD NFFFNCTLRH GMTPTDLEGQ LAGKGATTSI |
| 481 | ASHIKAPAAS KGAKASNGKP MAIYYGSNSG TCEALANRLA SDAAGHGFSA SVIGTLDQAK |
| 541 | QNLPEDRPVV IVTASYEGQP PSNAAHFIKW MEDLAGNEME KVSYAVFGCG HHDWVDTFLR |
| 601 | IPKLVDTTLE QRGGTRLVPM GSADAATSDM FSDFEAWEDT VLWPSLKEKY NVTDDEASGQ |
| 661 | RGLLVEVTTP RKTTLRQDVE EALVVSEKTL TKTGPAKKHI EIQLPSGMTY KAGDYLAILP |
| 721 | LNPRKTVSRV FRRFSLAWDS FLKIQSDGPT TLPINIAISA FDVFSAYVEL SQPATKRNIL |
| 781 | ALSEATEDKA TIQELEKLAG DAYQEDVSAK KVSVLDLLEK YPAVALPISS YLAMLPPMRV |
| 841 | RQYSISSSPF ADPSKLTLTY SLLDAPSLSG QGRHVGVATN FLSQLIAGDK LHISVRASSA |
| 901 | AFHLPSDPET TPIICVAAGT GLAPFRGFIQ ERAAMLAAGR KLAPALLFFG CRDPENDDLY |
| 961 | AEELARWEQM GAVDVRRAYS RATDKSEGCK YVQDRIYHDR ADVFKVWDQG AKVFICGSRE |
| 1021 | IGKAVEDICV RLAMERSEAT QEGKGATEEK AREWFERSRN ERFATDVFD |
| CYP505C2 | |
| G. zeae PH1a | |
| GenBank Accession No. EAA77183 | |
| >gi\|42554340\|gb\|EAA77183.1\| hypothetical protein FG07596.1 | |
| [Gibberella zeae PH-1] | |
| SEQ ID NO: 39 | |
| 1 | MAIKDGGKKS GQIPGPKGLP VLGNLFDLDL SDSLTSLINI GQKYAPIFSL ELGGHREVMI |
| 61 | CSRDLLDELC DETRFHKIVT GGVDKLRPLA GDGLFTAQHG NHDWGIAHRI LMPLFGPLKI |
| 121 | REMFDDMQDV SEQLCLKWAR LGPSATIDVA NDFTRLTLDT IALCTMGYRF NSFYSNDKMH |
| 181 | PFVDSMVAAL IDADKQSMFP DFIGACRVKA LSAFRKHAAI MKGTCNELIQ ERRKNPIEGT |
| 241 | DLLTAMMEGK DPKTGEGMSD DLIVQNLITF LIAGHETTSG LLSFAFYYLL ENPHTLEKAR |
| 301 | AEVDEVVGDQ ALNVDHLTKM PYVNMILRET LRLMPTAPGF FVTPHKDEII GGKYAVPANE |
| 361 | SLFCFLHLIH RDPKVWGADA EEFRPERMAD EFFEALPKNA WKPFGNGMRG CIGREFAWQE |
| 421 | AKLITVMILQ NFELSKADPS YKLKIKQSLT IKPDGFNMHA KLRNDRKVSG LFKAPSLSSQ |
| 481 | QPSLSSRQSI NAINAKDLKP ISIFYGSNTG TCEALAQKLS ADCVASGFMP SKPLPLDMAT |
| 541 | KNLSKDGPNI LLAASYDGRP SDNAEEFTKW AESLKPGELE GVQFAVFGCG HKDWVSTYFK |
| 601 | IPKILDKCLA DAGAERLVEI GLTDASTGRL YSDFDDWENQ KLFTELSKRQ GVTPTDDSHL |
| 661 | ELNVTVIQPQ NNDMGGNFKR AEVVENTLLT YPGVSRKHSL LLKLPKDMEY TPGDHVLVLP |
| 721 | KNPPQLVEQA MSCFGVDSDT ALTISSKRPT FLPTDTPILI SSLLSSLVEL SQTVSRTSLK |
| 781 | RLADFADDDD TKACVERIAG DDYTVEVEEQ RMSLLDILRK YPGINMPLST FLSMLPQMRP |
| 841 | RTYSFASAPE WKQGHGMLLF SVVEAEEGTV SRPGGLATNY MAQLRQGDSI LVEPRPCRPE |
| 901 | LRTTMMLPEP KVPIIMIAVG AGLAPFLGYL QKRFLQAQSQ RTALPPCTLL FGCRGAKMDD |
| 961 | ICRAQLDEYS RAGVVSVHRA YSRDPDSQCK YVQGLVTKHS ETLAKQWAQG AIVMVCSGKK |
| 1021 | VSDGVMNVLS PILFAEEKRS GMTGADSVDV WRQNVPKERM ILEVFG |
| CYP505A5 | |
| M. grisea 70-15 syn | |
| GenBank Accession No. XP 365223 | |
| >gi\|145601517\|ref\|XP_365223.2\| hypothetical protein MGG 01925 | |
| [Magnaporthe oryzae 70-15] | |
| SEQ ID NO: 40 | |
| 1 | MFFLSSSLAY MAATQSRDWA SFGVSLPSTA LGRHLQAAMP FLSEENHKSQ GTVLIPDAQG |
| 61 | PIPFLGSVPL VDPELPSQSL QRLARQYGEI YRFVIPGRQS PILVSTHALV NELCDEKRFK |
| 121 | KKVAAALLGL REAIHDGLFT AHNDEPNWGI AHRILMPAFG PMAIKGMFDE MHDVASQMIL |
| 181 | KWARHGSTTP IMVSDDFTRL TLDTIALCSM GYRFNSFYHD SMHEFIEAMT CWMKESGNKT |
| 241 | RRLLPDVFYR TTDKKWHDDA EILRRTADEV LKARKENPSG RKDLLTAMIE GVDPKTGGKL |
| 301 | SDSSIIDNLI TFLIAGHETT SGMLSFAFYL LLKNPTAYRK AQQEIDDLCG REPITVEHLS |
| 361 | KMPYITAVLR ETLRLYSTIP AFVVEAIEDT VVGGKYAIPK NHPIFLMIAE SHRDPKVYGD |
| 421 | DAQEFEPERM LDGQFERRNR EFPNSWKPFG NGMRGCIGRA FAWQEALLIT AMLLQNFNFV |
| 481 | MHDPAYQLSI KENLTLKPDN FYMRAILRHG MSPTELERSI SGVAPTGNKT PPRNATRTSS |
| 541 | PDPEDGGIPM SIYYGSNSGT CESLAHKLAV DASAQGFKAE TVDVLDAANQ KLPAGNRGPV |
| 601 | VLITASYEGL PPDNAKHFVE WLENLKGGDE LVDTSYAVFG CGHQDWTKTF HRIPKLVDEK |
| 661 | LAEHGAVRLA PLGLSNAAHG DMFVDFETWE FETLWPALAD RYKTGAGRQD AAATDLTAAL |
| 721 | SQLSVEVSHP RAADLRQDVG EAVVVAARDL TAPGAPPKRH MEIRLPKTGG RVHYSAGDYL |
| 781 | AVLPVNPKST VERAMRRFGL AWDAHVTIRS GGRTTLPTGA PVSAREVLSS YVELTQPATK |
| 841 | RGIAVLAGAV TGGPAAEQEQ AKAALLDLAG DSYALEVSAK RVGVLDLLER FPACAVPFGT |
| 901 | FLALLPPMRV RQYSISSSPL WNDEHATLTY SVLSAPSLAD PARTHVGVAS SYLAGLGEGD |
| 961 | HLHVALRPSH VAFRLPSPET PVVCVCAGSG MAPFRAFAQE RAALVGAGRK VAPLLLFFGC |
| 1021 | REPGVDDLYR EELEGWEAKG VLSVRRAYSR RTEQSEGCRY VQDRLLKNRA EVKSLWSQDA |
| 1081 | KVFVCGSREV AEGVKEAMFK VVAGKEGSSE EVQAWYEEVR NVRYASDIFD |
| CYP505A2 | |
| N. crassa OR74 A | |
| GenBank Accession No. XP 961848 | |
| >gi\|85104987\|ref\|XP_961848.1\| bifunctional P-450:NADPH-P450 reductase | |
| [Neurospora crassa OR74A] | |
| SEQ ID NO: 41 | |
| 1 | MSSDETPQTI PIPGPPGLPL VGNSFDIDTE FPLGSMLNFA DQYGEIFRLN FPGRNTVFVT |
| 61 | SQALVHELCD EKRFQKTVNS ALHEIRHGIH DGLFTARNDE PNWGIAHRIL MPAFGPMAIQ |
| 121 | NMFPEMHEIA SQLALKWARH GPNQSIKVTD DFTRLTLDTI ALCSMDYRFN SYYHDDMHPF |
| 181 | IDAMASFLVE SGNRSRRPAL PAFMYSKVDR KFYDDIRVLR ETAEGVLKSR KEHPSERKDL |
| 241 | LTAMLDGVDP KTGGKLSDDS IIDNLITFLI AGHETTSGLL SFAFVQLLKN PETYRKAQKE |
| 301 | VDDVCGKGPI KLEHMNKLHY IAAVLRETLR LCPTIPVIGV ESKEDTVIGG KYEVSKGQPF |
| 361 | ALLFAKSHVD PAVYGDTAND FDPERMLDEN FERLNKEFPD CWKPFGNGMR ACIGRPFAWQ |
| 421 | EALLVMAVCL QNFNFMPEDP NYTLQYKQTL TTKPKGFYMR AMLRDGMSAL DLERRLKGEL |
| 481 | VAPKPTAQGP VSGQPKKSGE GKPISIYYGS NTGTCETFAQ RLASDAEAHG FTATIIDSLD |
| 541 | AANQNLPKDR PVVFITASYE GQPPDNAALF VGWLESLTGN ELEGVQYAVF GCGHHDWAQT |
| 601 | FHRIPKLVDN TVSERGGDRI CSLGLADAGK GEMFTEFEQW EDEVFWPAME EKYEVSRKED |
| 661 | DNEALLQSGL TVNFSKPRSS TLRQDVQEAV VVDAKTITAP GAPPKRHIEV QLSSDSGAYR |
| 721 | SGDYLAVLPI NPKETVNRVM RRFQLAWDTN ITIEASRQTT ILPTGVPMPV HDVLGAYVEL |
| 781 | SQPATKKNIL ALAEAADNAE TKATLRQLAG PEYTEKITSR RVSILDLLEQ FPSIPLPFSS |
| 841 | FLSLLPPMRV RQYSISSSPL WNPSHVTLTY SLLESPSLSN PDKKHVGVAT SYLASLEAGD |
| 901 | KLNVSIRPSH KAFHLPVDAD KTPLIMIAAG SGLAPFRGFV QERAAQIAAG RSLAPAMLFY |
| 961 | GCRHPEQDDL YRDEFDKWES IGAVSVRRAF SRCPESQETK GCKYVGDRLW EDREEVTGLW |
| 1021 | DRGAKVYVCG SREVGESVKK VVVRIALERQ KMIVEAREKG ELDSLPEGIV EGLKLKGLTV |
| 1081 | EDVEVSEERA LKWFEGIRNE RYATDVFD |
| CYP97C | |
| Oryza sativa | |
| GenBank Accession No. ABB47954 | |
| >gi\|78708979\|gb\|ABB47954.1\| Cytochrome P450 family protein, expressed | |
| [Oryza sativa Japonica Group] | |
| SEQ ID NO: 42 | |
| 1 | MAAAAAAAVP CVPFLCPPPP PLVSPRLRRG HVRLRLRPPR SSGGGGGGGA GGDEPPITTS |
| 61 | WVSPDWLTAL SRSVATRLGG GDDSGIPVAS AKLDDVRDLL GGALFLPLFK WFREEGPVYR |
| 121 | LAAGPRDLVV VSDPAVARHV LRGYGSRYEK GLVAEVSEFL FGSGFAIAEG ALWTVRRRSV |
| 181 | VPSLHKRFLS VMVDRVFCKC AERLVEKLET SALSGKPVNM EARFSQMTLD VIGLSLFNYN |
| 241 | FDSLTSDSPV IDAVYTALKE AELRSTDLLP YWKIDLLCKI VPRQIKAEKA VNIIRNTVED |
| 301 | LITKCKKIVD AENEQIEGEE YVNEADPSIL RFLLASREEV TSVQLRDDLL SMLVAGHETT |
| 361 | GSVLTWTIYL LSKDPAALRR AQAEVDRVLQ GRLPRYEDLK ELKYLMRCIN ESMRLYPHPP |
| 421 | VLIRRAIVDD VLPGNYKIKA GQDIMISVYN IHRSPEVWDR ADDFIPERFD LEGPVPNETN |
| 481 | TEYRFIPFSG GPRKCVGDQF ALLEAIVALA VVLQKMDIEL VPDQKINMTT GATIHTTNGL |
| 541 | YMNVSLRKVD REPDFALSGS R |

Chimeric heme enzyme C2G9
SEQ ID NO: 43
MKETSPIPQPKTFGPLGNLPLIDKDKPTLSLIKLAEEQGPIFQIHTPAGTTIVVSGHELVKEVCDEERFDKSIEGAL
EKVRAFSGDGLATSWTHEPNWRKAHNILMPTESQRAMKDYHEKMVDIAVQLIQKWARLNPNEAVDVPGDMTRLTLDT
IGLCGENYRENSYYRETPHPFINSMVRALDEAMHQMQRLDVQDKLMVRTKRQFRYDIQTMESLVDRMIAERKANPDE
NIKDLLSLMLYAKDPVTGETLDDENIRYQIITFLIAGHETTSGLLSFALYELVKNPHVLQKAAEEAARVLVDPVPSY
KQVKQLKYVGMVLNEALRLWPTAPAFSLYAKEDTVLGGEYPISKGQPVTVLIPKLHRDQNAWGPDAEDFRPERFEDP
SSIPHHAYKPFGNGQRACIGMQFALHEATLVLGMILKYFTLIDHENYELDIKQTLTLKPGDFHISVQSRHQEAIHAD
VQAAE
Chimeric heme enzyme X7
SEQ ID NO: 44
MKETSPIPQPKTFGPLGNLPLIDKDKPTLSLIKLAEEQGPIFQIHTPAGTTIVVSGHELVKEVCDEERFDKSIEGAL
EKVRAFSGDGLATSWTHEPNWRKAHNILMPTESQRAMKDYHEKMVDIATQLIQKWSRLNPNEEIDVADDMTRLTLDT
IGLCGFNYRFNSFYRDQPHPFITSMVRALDEAMNKLQRANPDDPAYDENKRQFQEDIKVMNDLVDSIIAERRANGDQ
DEKDLLARMLNVEDPETGEKLDDENIRFQIITFLIAGHETTSGLLSFAIYCLLTHPEKLKKAQEEADRVLTDDTPEY
KQIQQLKYIRMVLNETLRLYPTAPAFSLYAKEDTVLGGEYPISKGQPVTVLIPKLHRDQNAWGPDAEDFRPERFEDP
SSIPHHAYKPFGNGQRACIGMQFALQEATMVLGLVLKHFELINHTGYELKIKEALTIKPDDFKITVKPRKTAAINVQ
RKEQA
Chimeric heme enzyme X7-12
SEQ ID NO: 45
MTIKEMPQPKTFGELKNLPLLNTDKPVQALMKIADELGEIFKFEAPGRVTRYLSSQRLIKEACDEERFDKSIEGALE
KVRAFSGDGLATSWTHEPNWRKAHNILMPTESQRAMKDYHEKMVDIAVQLVQKWERLNADEHIEVPEDMTRLTLDTI
GLCGFNYRFNSFYRDQPHPFITSMVRALDEAMNKLQRANPDDPAYDENKRQFQEDIKVMNDLVDSIIAERRANGDQD
EKDLLARMLNVEDPETGEKLDDENIRFQIITFLIAGHETTSGLLSFAIYCLLTHPEKLKKAQEEADRVLTDDTPEYK
QIQQLKYIRMVLNETLRLYPTAPAFSLYAKEDTVLGGEYPISKGQPVTVLIPKLHRDQNAWGPDAEDFRPERFEDPS
SIPHHAYKPFGNGQRACIGMQFALQEATMVLGLVLKHFELINHTGYELKIKEALTIKPDDFKITVKPRKTAAINVQR
KEQA
Chimeric heme enzyme C2E6
SEQ ID NO: 46
MTIKEMPQPKTFGELKNLPLLNTDKPVQALMKIADELGEIFKFEAPGRVTRYLSSQRLIKEACDESRFDKNLSQALK
FVRDFAGDGLFTSWTHEKNWKKAHNILLPSFSQQAMKGYHAMMVDIAVQLVQKWERLNADEHIEVPEDMTRLTLDTI
GLCGFNYRFNSFYRDQPHPFITSMVRALDEAMNKLQRANPDDPAYDENKRQFQEDIKVMNDLVDRMIAERKANPDEN
IKDLLSLMLYAKDPVTGETLDDENIRYQIITFLIAGHETTSGLLSFAIYCLLTHPEKLKKAQEEADRVLTDDTPEYK
QIQQLKYIRMVLNETLRLYPTAPAFSLYAKEDTVLGGEYPLEKGDELMVLIPQLHRDKTIWGDDVEEFRPERFENPS
AIPQHAFKPFGNGQRACIGQQFALHEATLVLGMMLKHFDFEDHTNYELDIKETLTLKPEGFVVKAKSKKIPLGGIPS
PST
Chimeric heme enzyme X7-9
SEQ ID NO: 47
MKQASAIPQPKTYGPLKNLPHLEKEQLSQSLWRIADELGPIFREDFPGVSSVFVSGHNLVAEVCDEERFDKSIEGAL
EKVRAFSGDGLATSWTHEPNWRKAHNILMPTESQRAMKDYHEKMVDIATQLIQKWSRLNPNEEIDVADDMTRLTLDT
IGLCGFNYRFNSFYRDQPHPFITSMVRALDEAMNKLQRANPDDPAYDENKRQFQEDIKVMNDLVDSIIAERRANGDQ
DEKDLLARMLNVEDPETGEKLDDENIRFQIITFLIAGHETTSGLLSFAIYCLLTHPEKLKKAQEEADRVLTDDTPEY
KQIQQLKYIRMVLNETLRLYPTAPAFSLYAKEDTVLGGEYPISKGQPVTVLIPKLHRDQNAWGPDAEDFRPERFEDP
SSIPHHAYKPFGNGQRACIGMQFALQEATMVLGLVLKHFELINHTGYELKIKEALTIKPDDFKITVKPRKTAAINVQ
RKEQA
Chimeric heme enzyme C2B12
SEQ ID NO: 48
MKQASAIPQPKTYGPLKNLPHLEKEQLSQSLWRIADELGPIFREDFPGVSSVFVSGHNLVAEVCDEERFDKSIEGAL
EKVRAFSGDGLATSWTHEPNWRKAHNILMPTESQRAMKDYHEKMVDIATQLIQKWSRLNPNEEIDVADDMTRLTLDT
IGLCGFNYRFNSFYRDQPHPFITSMVRALDEAMNKLQRANPDDPAYDENKRQFQEDIKVMNDLVDRMIAERKANPDE
NIKDLLSLMLYAKDPVTGETLDDENIRYQIITFLIAGHETTSGLLSFATYFLLKHPDKLKKAYEEVDRVLTDAAPTY
KQVLELTYIRMILNESLRLWPTAPAFSLYAKEDTVLGGEYPISKGQPVTVLIPKLHRDQNAWGPDAEDFRPERFEDP
SSIPHHAYKPFGNGQRACIGMQFALQEATMVLGLVLKHFELINHTGYELKIKEALTIKPDDFKITVKPRKTAAINVQ
RKEQA
Chimeric heme enzyme TSP234
SEQ ID NO: 49
MKETSPIPQPKTFGPLGNLPLIDKDKPTLSLIKLAEEQGPIFQIHTPAGTTIVVSGHELVKEVCDEERFDKSIEGAL
EKVRAFSGDGLATSWTHEPNWRKAHNILMPTESQRAMKDYHEKMVDIATQLIQKWSRLNPNEEIDVADDMTRLTLDT
IGLCGFNYRFNSFYRDQPHPFITSMVRALDEAMNKLQRANPDDPAYDENKRQFQEDIKVMNDLVDRMIAERKANPDE
NIKDLLSLMLYAKDPVTGETLDDENIRYQIITFLIAGHETTSGLLSFAIYCLLTHPEKLKKAQEEADRVLTDDTPEY
KQIQQLKYIRMVLNETLRLYPTAPAFSLYAKEDTVLGGEYPISKGQPVTVLIPKLHRDQNAWGPDAEDFRPERFEDP
SSIPHHAYKPFGNGQRACIGMQFALQEATMVLGLVLKHFELINHTGYELKIKEALTIKPDDFKITVKPRKTAAINVQ
RKEQA

1. A reaction mixture for producing an aziridination product, the reaction mixture comprising of an olefinic substrate, a nitrene precursor, and a heme enzyme.

2. The reaction mixture of claim 1, wherein the olefinic substrate is represented by a structure of Formula I: 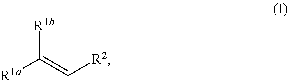
wherein:
R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl;
Y¹ is C₁₋₈alkylene;
each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen;
wherein each aryl contains between 6-14 carbon atoms, each heteroaryl group has from 5 to 8 ring atoms and from 1-3 heteroatoms selected from N, O and S, and each heterocyclyl group has from 1-3 heteroatoms selected from N, O and S.

3. The reaction mixture of claim 2, wherein
R^{1a}, R^{1b}, and R² are independently selected from the group consisting of H, C₁₋₁₈alkyl, aryl, heteroaryl, C₁₋₁₂cycloalkyl, and C₃₋₁₀heterocyclyl, and
each R^{1a}, R^{1b}, and R² is optionally substituted with from 1 to 5 substituents independently selected from the group consisting of C₁₋₃alkyl, alkoxy, and halogen.

4. The reaction mixture of claim 1, wherein the nitrene precursor has a formula selected from the group consisting of: 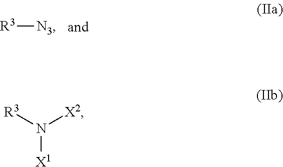
wherein
R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c};
X¹ is independently selected from the group consisting of H and sodium, and
X² is independently selected from the group consisting of halogen, —SO₂R^{a}, —CO₂R^{b}, —PO₃R^{b}R^{c}, optionally X¹ and X² can be taken together to form iodinane;
R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl;
R^{b} and R^{c} are independently selected from the group consisting of C₁₋₈alkyl, C₃₋₁₂cycloalkyl, aryl, heteroaryl, and C₃₋₈heterocyclyl;
wherein within each R³, R^{a}, R^{b}, and R^{c} can be optionally substituted with from 1-5 R^{d} substituents;
each R^{d} is independently selected from the group consisting of C₁₋₃alkyl, halogen, and hydroxy; and
wherein each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

5. The reaction mixture of claim 4, wherein the nitrene precursor is selected from the group consisting of:

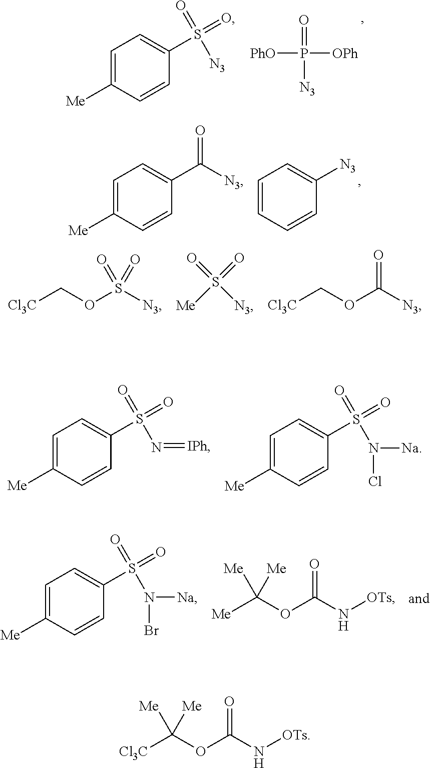

6. The reaction mixture of claim 5, wherein the nitrene precursor is

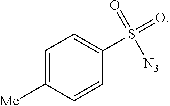 [SMILES: Cc1ccc(S(=O)(=O)N=[N+]=[N-])cc1]

7. The reaction mixture of claim 1, wherein the aziridination product is produced in vitro.

8. The reaction mixture of claim 7, wherein the reaction mixture further comprises a reducing agent.

9. The reaction mixture of claim 8, wherein the reducing agent is NADPH.

10. The reaction mixture of claim 1, wherein the heme enzyme is localized within a whole cell and the aziridination product is produced in vivo.

11. The reaction mixture of claim 10, wherein the whole cell is a bacterial cell or a yeast cell.

12. The reaction mixture of claim 1, wherein the aziridination product is produced under anaerobic conditions.

13. The reaction mixture of claim 1, wherein the heme enzyme is a variant thereof comprising a mutation at the axial position of the heme coordination site.

14. The reaction mixture of claim 13, wherein the heme enzyme comprises a serine mutation at the axial position of the heme coordination site.

15. The reaction mixture of claim 1, wherein the heme enzyme is a cytochrome P450 enzyme or a variant thereof.

16. The reaction mixture of claim 15, wherein the cytochrome P450 enzyme is a P450 BM3 enzyme or a variant thereof.

17. The reaction mixture of claim 16, wherein the P450 BM3 enzyme comprises an axial ligand mutation C400S and one or more mutations selected from the group consisting of V78, F87, P142, T175, A184, S226, H236, E252, I263, T268, A290, A328, L353, I366, L437, T438, and E442 relative to the amino acid sequence set forth in SEQ ID NO:1 (SEQ ID NO: 50).

18. The reaction mixture of claim 17, wherein the P450 BM3 enzyme comprises an axial ligand mutation C400S and mutations V78A, F87V, P142S, T175I, A184V, S226R, H236Q, E252G, I263F, T268A, A290V, A328V, L353V, I366V, L437V, T438S, and E442K relative to the amino acid sequence set forth in SEQ ID NO:1 (SEQ ID NO: 51).

19. The reaction mixture of claim 16, wherein the P450 BM3 enzyme comprises an axial ligand mutation C400S and one or more mutations selected from the group consisting of L75, V78, F87, P142, T175, L181, A184, S226, H236, E252, I263, T268, A290, L353, I366, and E442 relative to the amino acid sequence set forth in SEQ ID NO: 1 (SEQ ID NO: 52).

20. The reaction mixture of claim 19, wherein the P450 BM3 enzyme comprises an axial ligand mutation C400S and mutations L75A, V87A, F87V, P142S, T175I, L181A, A184V, S226R, H236Q, E252G, I263F, T268A, A290V, L353V, I366V, and E442K relative to the amino acid sequence set forth in SEQ ID NO:1 (SEQ ID NO: 53).

21. The reaction mixture of claim 1, wherein the aziridination product is an aziridine compound according to Formula III: 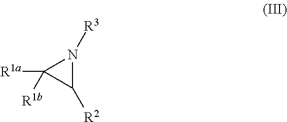
wherein
R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl;
Y¹ is C₁₋₈alkylene;
each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen;
R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c};
R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl;
R^{b} and R^{c} are independently selected from the group consisting of C₁₋₈alkyl, C₃₋₁₂cycloalkyl, aryl, heteroaryl, and C₃₋₈heterocyclyl;
wherein within each R³, R^{a}, R^{b}, and R^{c} can be optionally substituted with from 1-5 R^{d} substituents;
each R^{d} is independently selected from the group consisting of C₁₋₃alkyl, halogen, and hydroxy; and
wherein each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

22. The reaction mixture of claim 21, wherein
R^{1a} and R^{1b} are independently selected from the group consisting of H, C₁₋₈alkyl, aryl, heteroaryl, C₁₋₁₂cycloalkyl, and C₃₋₁₀heterocyclyl;
R² is selected from the group consisting of H and C₁₋₈ alkyl;
each R^{1a}, R^{1b}, and R² is optionally substituted with from 1 to 3 substituents independently selected from the group consisting of C₁₋₃alkyl, alkoxy, and halogen; and
R³ is selected from the group consisting of —SO₂R^{a}, —COR^{a}, —CO₂R^{b}, —PO₃R^{b}R^{c}, and —CONR^{b}R^{c},
R^{a} is independently selected from the group consisting of C₃₋₁₂cycloalkyl, aryl, heteroaryl, and C₃₋₈heterocyclyl;
R^{b} and R^{c} are independently selected from the group consisting of C₃₋₁₂cycloalkyl, aryl, heteroaryl, and C₃₋₈heterocyclyl;
wherein within each R³, R^{a}, R^{b}, and R^{c} can be optionally substituted with from 1-2 R^{d} substituents; and
each R^{d} is independently selected from the group consisting of C₁₋₃alkyl, halogen, and hydroxy.

23. The reaction mixture of claim 1, wherein the aziridination product is an amido-alcohol compound according to Formula IIIa: 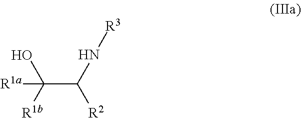
wherein
R^{1a}, R^{1b}, and R² are independently selected from the group consisting of H, C₁₋₁₈alkyl, C₁₋₈heteroalkyl, aryl, heteroaryl, C₁₋₁₂cycloalkyl, C₃₋₁₀heterocyclyl, —Y¹-aryl, —Y¹-heteroaryl, —Y¹—C₁₋₁₂cycloalkyl and —Y¹—C₃₋₁₀heterocyclyl;
Y¹ is C₁₋₈alkylene;
each R^{1a}, R^{1b}, and R² is optionally substituted with from 1 to 5 substituents independently selected from the group consisting of C₁₋₃alkyl, alkoxy hydroxyl, amino, thiol, carboxy, amido, oxo, thioxo, cyano, and halogen;
R³ is selected from the group consisting of C₁₋₁₈ alkyl, C₁₋₈heteroalkyl, C₃₋₁₂cycloalkyl, aryl, heteroaryl, C₃₋₁₀heterocyclyl, —SO₂R^{a}, —COR^{a}, —CO₂R^{b}, —PO₃R^{b}R^{c}, and —CONR^{b}R^{c};
R^{a} is independently selected from the group consisting of C₁₋₈alkyl, hydroxy, C₁₋₈alkoxy, C₃₋₁₂cycloalkyl, aryl, heteroaryl, and C₃₋₈heterocyclyl;
R^{b} and R^{c} are independently selected from the group consisting of C₁₋₈alkyl, C₃₋₁₂cycloalkyl, aryl, heteroaryl, and C₃₋₈heterocyclyl;
wherein within each R³, R^{a}, R^{b}, and R^{c} can be optionally substituted with from 1-5 R^{d} substituents;
each R^{d} is independently selected from the group consisting of C₁₋₃alkyl, halogen, and hydroxy; and
wherein each aryl contains between 6-14 carbon atoms, each heteroaryl group has from 5 to 10 ring atoms and from 1-3 heteroatoms selected from N, O and S, and each heterocyclyl group has from 1-3 heteroatoms selected from N, O and S.

24. The reaction mixture of claim 23, wherein
R^{1a} and R^{1b}, are independently selected from the group consisting of H, C₁₋₈alkyl, aryl, heteroaryl;
R² is selected from the group consisting of H, and C₁₋₈ alkyl,
each R^{1a}, R^{1b}, and R² is optionally substituted with from 1 to 3 substituents independently selected from the group consisting of C₁₋₃alkyl, alkoxy and halogen; and
R³ is selected from the group consisting of —SO₂R^{a}, —COR^{a}, —CO₂R^{b}, —PO₃R^{b}R^{c}, and —CONR^{b}R^{c},
R^{a} is independently selected from the group consisting of C₃₋₁₂cycloalkyl, aryl, heteroaryl, and C₃₋₈heterocyclyl;
R^{b} and R^{c} are independently selected from the group consisting of C₃₋₁₂cycloalkyl, aryl, heteroaryl, and C₃₋₈heterocyclyl;
wherein within each R³, R^{a}, R^{b}, and R^{c} can be optionally substituted with from 1-2 R^{d} substituents; and
each R^{d} is independently selected from the group consisting of C₁₋₃alkyl, halogen, and hydroxy.

25. The reaction mixture of claim 1, wherein the reaction produces a plurality of aziridination products.

26. The reaction mixture of claim 25, wherein the plurality of aziridination products has a % ee_{S} of from about −99% to about 99%.

27. The reaction mixture of claim 25, wherein the plurality of aziridination products has a % ee_{S} of from about −86% to about 86%.

28. The reaction mixture of claim 25, wherein the plurality of aziridination products has a Z:E ratio of from about 1:99 to about 99:1.

29. The reaction mixture of claim 25, wherein the reaction is at least 30% to at least 90% diastereoselective.

30-49. (canceled)


SEQUENCE LISTING (57 sequences)

SEQ ID NO: 1 - protein, 1048 aa, Bacillus megaterium
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 2 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 3 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKEFVDWLDQASADEVKGVRYSVFGCGDKNWATTYQKVPAFIDETFAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 4 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQPGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSIRPRYYSISSSPRVDEKQASITVSVVSGEAWSGYGEYKGIASNYLANLQEGDTITCFVSTPQSGFTLPKDSETPLIMVGPGTGVAPFRSFVQARKQLKEQGQSLGEAHLYFGCRSPHEDYLYQEELENAQNEGIITLHTAFSRVPNQPKTYVQHVMEQDGKKLIELLDQGAHFYICGDGSQMAPDVEATLMKSYADVYEVSEADARLWLQQLEEKGRYAKDVWAG

SEQ ID NO: 5 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPRVATLDSHAGNLPREGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNQPKTYVQHVMEQDGKKLIELLDQGAHFYICGDGSQMAPDVEATLMKSYAEVHQVSEADARLWLQQLEEKGRYAKDVWAG

SEQ ID NO: 6 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 7 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPPEGAVLIVTASYNGHPPDNAKEFVDWLDQASADEVKGVRYSVFGCGDKNWATTYQKVPAFIDETLAAKGAENIAERGEADASDDFEGTYEEWREHMWSDLAAYFNLDIENSEENASTLSLQFVDSAADMPLAKMHRAFSANVVASKELQKPGSARSTRHLEIELPKEASYQEGDHLGVIPRNYEGIVNRVATRFGLDASQQIRLEAEEEKLAHLPLGKTVSVEELLQYVELQDPVTRTQLRAMAAKTVCPPHKVELEVLLEKQAYKEQVLAKRLTMLELLEKYPACEMEFSEFIALLPSMRPRYYSISSSPRVDEKQASITVSVVSGEAWSGYGEYKGIASNYLANLQEGDTITCFVSTPQSGFTLPKGPETPLIMVGPGTGVAPFRGFVQARKQLKEQGQSLGEAHLYFGCRSPHEDYLYQKELENAQNEGIITLHTAFSRVPNEPKTYVQHVMEQDGKKLIELLDQGAHFYICGDGSQMAPDVEATLMKSYAEVHQVSEADARLWLQQLEEKGRYAKDVWAG

SEQ ID NO: 8 - protein, 1049 aa, Bacillus megaterium
MTIKEMPQPKTFGELKNLPLLNTDKPIQTLMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNTDEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEATRVLVDPVPSYKQVKQLKYVGMVLNEALRLWPTAPAFSLYAKEDTVLGGEYPLEKGDELMVLIPQLHRDKTIWGEDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKVENAHNTPLLVLYGSNMGTAEGTARDLADIAMSKGFAPQVATLDSHAGNLPREGAVLIVTASYNGHPPDNAKQFVDWLDQASADDVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSANVVASKELQQLGSERSTRHLEIALPKEASYQEGDHLGVIPRNYEGIVNRVTARFGLDASQQIRLEAEEEKLAHLPLGKTVSVEELLQYVELQDPVTRTQLRAMAAKTVCPPHKVELEALLEKQAYKEQVLAKRLTMLELLEKYPACEMEFSEFIALLPSISPRYYSISSSPHVDEKQASITVSVVSGEAWSGYGEYKGIASNYLANLQEGDTITCFVSTPQSGFTLPKDSETPLIMVGPGTGVAPFRGFVQARKQLKEQGQSLGEAHLYFGCRSPHEDYLYQEELENAQNEGIITLHTAFSRVPNQPKTYVQHVMERDGKKLIELLDQGAHFYICGDGSQMAPDVEATLMKSYADVYEVSEADARLWLQQLEEKGRYAKDVWAG

SEQ ID NO: 9 - protein, 1049 aa, Bacillus megaterium; identical to SEQ ID NO: 2

SEQ ID NO: 10 - protein, 1049 aa, Bacillus megaterium
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLIQKWERLNADEHIEVPEDMTRLTLDTIGLCGFNYRFNSFYRDQPHPFITSMVRALDEAMNKLQRANPDDPAYDENKRQFQDDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKQIPLGGIPSPSREQSAKKERKTVENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLSAKGAENIAERGEADASDDFEGTYEEWREHMWSDLAAYFNLNIENSEDNASTLSLQFVDSAADMPLAKMHGAFSANVVASKELQQPGSARSTRHLEIELPKEASYQEGDHLGVIPRNYEGIVNRVTTRFGLDASQQIRLEAEEEKLAHLPLGKTVSVEELLQYVELQDPVTRTQLRAMAAKTVCPPHKVELEALLEKQAYKEQVLTKRLTMLELLEKYPACEMEFSEFIALLPSMRPRYYSISSSPRVDEKQASITVSVVSGEAWSGYGEYKGIASNYLAELQEGDTITCFVSTPQSGFTLPKDPETPLIMVGPGTGVAPFRGFVQARKQLKEQGQSLGEAHLYFGCRSPHEDYLYQEELENAQNEGIITLHTAFSRVPNQPKTYVQHVVEQDGKKLIELLDQGAHFYICGDGSQMAPDVEATLMKSYAEVHKVSEADARLWLQQLEEKSRYAKDVWAG

SEQ ID NO: 11 - protein, 1049 aa, Bacillus megaterium; identical to SEQ ID NO: 10

SEQ ID NO: 12 - protein, 420 aa, Mycobacterium sp. HXN-1500
MTEMTVAASDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIELTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINA

SEQ ID NO: 13 - protein, 496 aa, Tetrahymena thermophile
MIFELILIAVALFAYFKIAKPYFSYLKYRKYGKGFYYPILGEMIEQEQDLKQHADADYSVHHALDKDPDQKLFVTNLGTKVKLRLIEPEIIKDFFSKSQYYQKDQTFIQNITRFLKNGIVFSEGNTWKESRKLFSPAFHYEYIQKLTPLINDITDTIFNLAVKNQELKNFDPIAQIQEITGRVIIASFFGEVIEGEKFQGLTIIQCLSHIINTLGNQTYSIMYFLFGSKYFELGVTEEHRKFNKFIAEFNKYLLQKIDQQIEIMSNELQTKGYIQNPCILAQLISTHKIDEITRNQLFQDFKTFYIAGMDTTGHLLGMTIYYVSQNKDIYTKLQSEIDSNTDQSAHGLIKNLPYLNAVIKETLRYYGPGNILFDRIAIKDHELAGIPIKKGTIVTPYAMSMQRNSKYYQDPHKYNPSRWLEKQSSDLHPDANIPFSAGQRKCIGEQLALLEARIILNKFIKMFDFTCPQDYKLMMNYKFLSEPVNPLPLQLTLRKQ

SEQ ID NO: 14 - protein, 394 aa, Nonomuraea dietziae
VNIDLVDQDHYATFGPPHEQMRWLREHAPVYWHEGEPGFWAVTRHEDVVHVSRHSDLFSSARRLALFNEMPEEQRELQRMMMLNQDPPEHTRRRSLVNRGFTPRTIRALEQHIRDICDDLLDQCSGEGDFVTDLAAPLPLYVICELLGAPVADRDKIFAWSNRMIGAQDPDYAASPEEGGAAAMEVYAYASELAAQRRAAPRDDIVTKLLQSDENGESLTENEFELFVLLLVVAGNETTRNAASGGMLTLFEHPDQWDRLVADPSLAATAADEIVRWVSPVNLFRRTATADLTLGGQQVKADDKVVVFYSSANRDASVFSDPEVFDIGRSPNPHIGFGGGGAHFCLGNHLAKLELRVLFEQLARRFPRMRQTGEARRLRSNFINGIKTLPVTLG

SEQ ID NO: 15 - protein, 501 aa, Homo sapiens
MWKLWRAEEGAAALGGALFLLLFALGVRQLLKQRRPMGFPPGPPGLPFIGNIYSLAASSELPHVYMRKQSQVYGEIFSLDLGGISTVVLNGYDVVKECLVHQSEIFADRPCLPLFMKMTKMGGLLNSRYGRGWVDHRRLAVNSFRYFGYGQKSFESKILEETKFFNDAIETYKGRPFDFKQLITNAVSNITNLIIFGERFTYEDTDFQHMIELFSENVELAASASVFLYNAFPWIGILPFGKHQQLFRNAAVVYDFLSRLIEKASVNRKPQLPQHFVDAYLDEMDQGKNDPSSTFSKENLIFSVGELIIAGTETTTNVLRWAILFMALYPNIQGQVQKEIDLIMGPNGKPSWDDKCKMPYTEAVLHEVLRFCNIVPLGIFHATSEDAVVRGYSIPKGTTVITNLYSVHFDEKYWRDPEVFHPERFLDSSGYFAKKEALVPFSLGRRHCLGEHLARMEMFLFFTALLQRFHLHFPHELVPDLKPRLGMTLQPQPYLICAERR

SEQ ID NO: 16 - protein, 501 aa, Macca mulatta
MWKLWGGEEGAAALGGALFLLLFALGVRQLLKLRRPMGFPPGPPGLPFIGNIYSLAASAELPHVYMRKQSQVYGEIFSLDLGGISTVVLNGYDVVKECLVHQSGIFADRPCLPLFMKMTKMGGLLNSRYGQGWVEHRRLAVNSFRYFGYGQKSFESKILEETKFFTDAIETYKGRPFDFKQLITSAVSNITNLIIFGERFTYEDTDFQHMIELFSENVELAASASVFLYNAFPWIGILPFGKHQQLFRNASVVYDFLSRLIEKASVNRKPQLPQHFVDAYFDEMDQGKNDPSSTFSKENLIFSVGELIIAGTETTTNVLRWAILFMALYPNIQGQVQKEIDLIMGPNGKPSWDDKFKMPYTEAVLHEVLRFCNIVPLGIFHATSEDAVVRGYSIPKGTTVITNLYSVHFDEKYWRDPEVFHPERFLDSSGYFAKKEALVPFSLGRRHCLGEQLARMEMFLFFTALLQRFHLHFPHELVPDLKPRLGMTLQPQPYLICAERR

SEQ ID NO: 17 - protein, 501 aa, Canis familiaris
MRGPPGAEACAAGLGAALLLLLFVLGVRQLLKQRRPAGFPPGPSGLPFIGNIYSLAASGELAHVYMRKQSRVYGEIFSLDLGGISAVVLNGYDVVKECLVHQSEIFADRPCLPLFMKMTKMGGLLNSRYGRGWVDHRKLAVNSFRCFGYGQKSFESKILEETNFFIDAIETYKGRPFDLKQLITNAVSNITNLIIFGERFTYEDTDFQHMIELFSENVELAASASVFLYNAFPWIGIIPFGKHQQLFRNAAVVYDFLSRLIEKASINRKPQSPQHFVDAYLNEMDQGKNDPSCTFSKENLIFSVGELIIAGTETTTNVLRWAILFMALYPNIQGQVQKEIDLIMGPTGKPSWDDKCKMPYTEAVLHEVLRFCNIVPLGIFHATSEDAVVRGYSIPKGTTVITNLYSVHFDEKYWRNPEIFYPERFLDSSGYFAKKEALVPFSLGKRHCLGEQLARMEMFLFFTALLQRFHLHFPHGLVPDLKPRLGMTLQPQPYLICAERR

SEQ ID NO: 18 - protein, 222 aa, Mus musculus
MGDEMDQGQNDPLSTFSKENLIFSVGELIIAGTETTTNVLRWAILFMALYPNIQGQVHKEIDLIVGHNRRPSWEYKCKMPYTEAVLHEVLRFCNIVPLGIFHATSEDAVVRGYSIPKGTTVITNLYSVHFDEKYWKDPDMFYPERFLDSNGYFTKKEALIPFSLGRRHCLGEQLARMEMFLFFTSLLQQFHLHFPHELVPNLKPRLGMTLQPQPYLICAERR

SEQ ID NO: 19 - protein, 422 aa, Bacillus halodurans C-125
MKSNDPIPKDSPLDHTMNLMREGYEFLSHRMERFQTDLFETRVMGQKVLCIRGAEAVKLFYDPERFKRHRATPKRIQKSLFGENAIQTMDDKAHLHRKQLFLSMMKPEDEQELARLTHETWRRVAEGWKKSRPIVLFDEAKRVLCQVACEWAEVPLKSTEIDRRAEDFHAMVDAFGAVGPRHWRGRKGRRRTERWIQSIIHQVRTGSLQAREGSPLYKVSYHRELNGKLLDERMAAIELINVLRPIVAIATFISFAAIALQEHPEWQERLKNGSNEEFHMFVQEVRRYYPFAPLIGAKVRKSFTWKGVRFKKGRLVFLDMYGTNHDPKLWDEPDAFRPERFQERKDSLYDFIPQGGGDPTKGHRCPGEGITVEVMKTTMDFLVNDIDYDVPDQDISYSLSRMPTRPESGYIMANIERKYEHA

SEQ ID NO: 20 - protein, 389 aa, Streptomyces parvus
MYLGGRRGTEAVGESREPGVWEVFRYDEAVQVLGDHRTFSSDMNHFIPEEQRQLARAARGNFVGIDPPDHTQLRGLVSQAFSPRVTAALEPRIGRLAEQLLDDIVAERGDKASCDLVGEFAGPLSAIVIAELFGIPESDHTMIAEWAKALLGSRPAGELSIADEAAMQNTADLVRRAGEYLVHHITERRARPQDDLTSRLATTEVDGKRLDDEEIVGVIGMFLIAGYLPASVLTANTVMALDEHPAALAEVRSDPALLPGAIEEVLRWRPPLVRDQRLTTRDADLGGRTVPAGSMVCVWLASAHRDPFRFENPDLFDIHRNAGRHLAFGKGIHYCLGAPLARLEARIAVETLLRRFERIEIPRDESVEFHESIGVLGPVRLPTTLFARR

SEQ ID NO: 21 - protein, 414 aa, Pseudomonas putida
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 22 - protein, 515 aa, Homo sapiens
GLEALVPLAMIVAIFLLLVDLMHRHQRWAARYPPGPLPLPGLGNLLHVDFQNTPYCFDQLRRRFGDVFNLQLAWTPVVVLNGLAAVREAMVTRGEDTADRPPAPIYQVLGFGPRSQGVILSRYGPAWREQRRFSVSTLRNLGLGKKSLEQWVTEEAACLCAAFADQAGRPFRPNGLLDKAVSNVIASLTCGRRFEYDDPRFLRLLDLAQEGLKEESGFLREVLNAVPVLPHIPALAGKVLRFQKAFLTQLDELLTEHRMTWDPAQPPRDLTEAFLAKKEKAKGSPESSFNDENLRIVVGNLFLAGMVTTLTTLAWGLLLMILHLDVQRGRRVSPGCSPIVGTHVCPVRVQQEIDDVIGQVRRPEMGDQVHMPYTTAVIHEVQRFGDIVPLGVTHMTSRDIEVQGFRIPKGTTLITNLSSVLKDEAVWEKPFRFHPEHFLDAQGHFVKPEAFLPFSAGRRACLGEPLARMELFLFFTSLLQHFSFSVAAGQPRPSHSRVVSFLVTPSPYELCAVPR

SEQ ID NO: 23 - protein, 532 aa, Rattus norvegicus
AVLSRMRLRWALLDTRVMGHGLCPQGARAKAAIPAALRDHESTEGPGTGQDRPRLRSLAELPGPGTLRFLFQLFLRGYVLHLHELQALNKAKYGPMWTTTFGTRTNVNLASAPLLEQVMRQEGKYPIRDSMEQWKEHRDHKGLSYGIFITQGQQWYHLRHSLNQRMLKPAEAALYTDALNEVISDFIARLDQVRTESASGDQVPDVAHLLYHLALEAICYILFEKRVGCLEPSIPEDTATFIRSVGLMFKNSVYVTFLPKWSRPLLPFWKRYMNNWDNIFSFGEKMIHQKVQEIEAQLQAAGPDGVQVSGYLHFLLTKELLSPQETVGTFPELILAGVDTTSNTLTWALYHLSKNPEIQEALHKEVTGVVPFGKVPQNKDFAHMPLLKAVIKETLRLYPVVPTNSRIITEKETEINGFLFPKNTQFVLCTYVVSRDPSVFPEPESFQPHRWLRKREDDNSGIQHPFGSVPFGYGVRSCLGRRIAELEMQLLLSRLIQKYEVVLSPGMGEVKSVSRIVLVPSKKVSLRFLQRQ

SEQ ID NO: 24 - protein, 491 aa, Oryctolagus cuniculus
MEFSLLLLLAFLAGLLLLLFRGHPKAHGRLPPGPSPLPVLGNLLQMDRKGLLRSFLRLREKYGDVFTVYLGSRPVVVLCGTDAIREALVDQAEAFSGRGKIAVVDPIFQGYGVIFANGERWRALRRFSLATMRDFGMGKRSVEERIQEEARCLVEELRKSKGALLDNTLLFHSITSNIICSIVFGKRFDYKDPVFLRLLDLFFQSFSLISSFSSQVFELFPGFLKHFPGTHRQIYRNLQEINTFIGQSVEKHRATLDPSNPRDFIDVYLLRMEKDKSDPSSEFHHQNLILTVLSLFFAGTETTSTTLRYGFLLMLKYPHVTERVQKEIEQVIGSHRPPALDDRAKMPYTDAVIHEIQRLGDLIPFGVPHTVTKDTQFRGYVIPKNTEVFPVLSSALHDPRYFETPNTFNPGHFLDANGALKRNEGFMPFSLGKRICLGEGIARTELFLFFTTILQNFSIASPVPPEDIDLTPRESGVGNVPPSYQIRFLAR

SEQ ID NO: 25 - protein, 1061 aa, Bacillus subtilis
MKETSPIPQPKTFGPLGNLPLIDKDKPTLSLIKLAEEQGPIFQIHTPAGTTIVVSGHELVKEVCDEERFDKSIEGALEKVRAFSGDGLFTSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRHDIQTMFSLVDSIIAERRANGDQDEKDLLARMLNVEDPETGEKLDDENIRFQIITFLIAGHETTSGLLSFATYFLLKHPDKLKKAYEEVDRVLTDAAPTYKQVLELTYIRMILNESLRLWPTAPAFSLYPKEDTVIGGKFPITTNDRISVLIPQLHRDRDAWGKDAEEFRPERFEHQDQVPHHAYKPFGNGQRACIGMQFALHEATLVLGMILKYFTLIDHENYELDIKQTLTLKPGDFHIRVQSRNQDAIHADVQAVEKAASDEQKEKTEAKGTSVIGLNNRPLLVLYGSDTGTAEGVARELADTASLHGVRTETAPLNDRIGKLPKEGAVVIVTSSYNGKPPSNAGQFVQWLQEIKPGELEGVHYAVFGCGDHNWASTYQYVPRFIDEQLAEKGATRFSARGEGDVSGDFEGQLDEWKKSMWADAIKAFGLELNENADKERSTLSLQFVRGLGESPLARSYEASHASIAENRELQSADSDRSTRHIEIALPPDVEYQEGDHLGVLPKNSQTNVSRILHRFGLKGTDQVTLSASGRSAGHLPLGRPVSLHDLLSYSVEVQEAATRAQIRELAAFTVCPPHRRELEELSAEGVYQEQILKKRISMLDLLEKYEACDMPFERFLELLRPLKPRYYSISSSPRVNPRQASITVGVVRGPAWSGRGEYRGVASNDLAERQAGDDVVMFIRTPESRFQLPKDPETPIIMVGPGTGVAPFRGFLQARDVLKREGKTLGEAHLYFGCRNDRDFIYRDELERFEKDGIVTVHTAFSRKEGMPKTYVQHLMADQADTLISILDRGGRLYVCGDGSKMAPDVEAALQKAYQAVHGTGEQEAQNWLRHLQDTGMYAKDVWAGI

SEQ ID NO: 26 - protein, 1054 aa, Bacillus subtilis
MKQASAIPQPKTYGPLKNLPHLEKEQLSQSLWRIADELGPIFRFDFPGVSSVFVSGHNLVAEVCDESRFDKNLGKGLQKVREFGGDGLFTSWTHEPNWQKAHRILLPSFSQKAMKGYHSMMLDIATQLIQKWSRLNPNEEIDVADDMTRLTLDTIGLCGFNYRFNSFYRDSQHPFITSMLRALKEAMNQSKRLGLQDKMMVKTKLQFQKDIEVMNSLVDRMIAERKANPDDNIKDLLSLMLYAKDPVTGETLDDENIRYQIITFLIAGHETTSGLLSFAIYCLLTHPEKLKKAQEEADRVLTDDTPEYKQIQQLKYTRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQADIKAETKPKETKPKHGTPLLVLYGSNLGTAEGIAGELAAQGRQMGFTAETAPLDDYIGKLPEEGAVVIVTASYNGSPPDNAAGFVEWLKELEEGQLKGVSYAVFGCGNRSWASTYQRIPRLIDDMMKAKGASRLTEIGEGDAADDFESHRESWENRFWKETMDAFDINEIAQKEDRPSLSIAFLSEATETPVAKAYGAFEGVVLENRELQTADSTRSTRHIELEIPAGKTYKEGDHIGIMPKNSRELVQRVLSRFGLQSNHVIKVSGSAHMSHLPMDRPIKVADLLSSYVELQEPASRLQLRELASYTVCPPHQKELEQLVLDDGIYKEQVLAKRLTMLDFLEDYPACEMPFERFLALLPSLKPRYYSISSSPKVHANIVSMTVGVVKASAWSGRGEYRGVASNYLAELNTGDAAACFIRTPQSGFQMPDEPETPMIMVGPGTGIAPFRGFIQARSVLKKEGSTLGEALLYFGCRRPDHDDLYREELDQAEQEGLVTIRRCYSRVENESKGYVQHLLKQDSQKLMTLIEKGAHIYVCGDGSQMAPDVEKTLRWAYETEKGASQEESADWLQKLQDQKRYIKDVWTGN

SEQ ID NO: 27 - protein, 1049 aa, B. megaterium DSM 32 [sequence omitted]

SEQ ID NO: 28 - protein, 1065 aa, Bacillus cereus
MEKKVSAIPQPKTYGPLGNLPLIDKDKPTLSFIKIAEEYGPIFQIQTLSDTIIVVSGHELVAEVCDETRFDKSIEGALAKVRAFAGDGLFTSETHEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSGDQEENDLLSRMLNVPDPETGEKLDDENIRFQIITFLIAGHETTSGLLSFAIYFLLKNPDKLKKAYEEVDRVLTDPTPTYQQVMKLKYMRMILNESLRLWPTAPAFSLYAKEDTVIGGKYPIKKGEDRISVLIPQLHRDKDAWGDNVEEFQPERFEELDKVPHHAYKPFGNGQRACIGMQFALHEATLVMGMLLQHFELIDYQNYQLDVKQTLTLKPGDFKIRILPRKQTISHPTVLAPTEDKLKNDEIKQHVQKTPSIIGADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSKRGEADASGDFEEQLEQWKQNMWSDAMKAFGLELNKNMEKERSTLSLQFVSRLGGSPLARTYEAVYASILENRELQSSSSDRSTRHIEVSLPEGATYKEGDHLGVLPVNSEKNINRILKRFGLNGKDQVILSASGRSINHIPLDSPVSLLALLSYSVEVQEAATRAQIREMVTFTACPPHKKELEALLEEGVYHEQILKKRISMLDLLEKYEACEIRFERFLELLPALKPRYYSISSSPLVAHNRLSITVGVVNAPAWSGEGTYEGVASNYLAQRHNKDEIICFIRTPQSNFELPKDPETPIIMVGPGTGIAPFRGFLQARRVQKQKGMNLGQAHLYFGCRHPEKDYLYRTELENDERDGLISLHTAFSRLEGHPKTYVQHLIKQDRINLISLLDNGAHLYICGDGSKMAPDVEDTLCQAYQEIHEVSEQEARNWLDRVQDEGRYGKDVWAGI

SEQ ID NO: 29 - protein, 1074 aa, Bacillus licheniformis
MNKLDGIPIPKTYGPLGNLPLLDKNRVSQSLWKIADEMGPIFQFKFADAIGVFVSSHELVKEVSEESRFDKNMGKGLLKVREFSGDGLFTSWTEEPNWRKAHNILLPSFSQKAMKGYHPMMQDIAVQLIQKWSRLNQDESIDVPDDMTRLTLDTIGLCGFNYRFNSFYREGQHPFIESMVRGLSEAMRQTKRFPLQDKLMIQTKRRFNSDVESMFSLVDRIIADRKQAESESGNDLLSLMLHAKDPETGEKLDDENIRYQIITFLIAGHETTSGLLSFAIYLLLKHPDKLKKAYEEADRVLTDPVPSYKQVQQLKYIRMILNESIRLWPTAPAFSLYAKEETVIGGKYLIPKGQSVTVLIPKLHRDQSVWGEDAEAFRPERFEQMDSIPAHAYKPFGNGQRACIGMQFALHEATLVLGMILQYFDLEDHANYQLKIKESLTLKPDGFTIRVRPRKKEAMTAMPGAQPEENGRQEERPSAPAAENTHGTPLLVLYGSNLGTAEEIAKELAEEAREQGFHSRTAELDQYAGAIPAEGAVIIVTASYNGNPPDCAKEFVNWLEHDQTDDLRGVKYAVFGCGNRSWASTYQRIPRLIDSVLEKKGAQRLHKLGEGDAGDDFEGQFESWKYDLWPLLRTEFSLAEPEPNQTETDRQALSVEFVNAPAASPLAKAYQVFTAKISANRELQCEKSGRSTRHIEISLPEGAAYQEGDHLGVLPQNSEVLIGRVFQRFGLNGNEQILISGRNQASHLPLERPVHVKDLFQHCVELQEPATRAQIRELAAHTVCPPHQRELEDLLKDDVYKDQVLNKRLTMLDLLEQYPACELPFARFLALLPPLKPRYYSISSSPQLNPRQTSITVSVVSGPALSGRGHYKGVASNYLAGLEPGDAISCFIREPQSGFRLPEDPETPVIMVGPGTGIAPYRGFLQARRIQRDAGVKLGEAHLYFGCRRPNEDFLYRDELEQAEKDGIVHLHTAFSRLEGRPKTYVQDLLREDAALLIHLLNEGGRLYVCGDGSRMAPAVEQALCEAYRIVQGASREESQSWLSALLEEGRYAKDVWDGGVSQHNVKADCIART

SEQ ID NO: 30 - protein, 1065 aa, Bacillus thuringiensis serovar konkukian
MDKKVSAIPQPKTYGPLGNLPLIDKDKPTLSFIKLAEEYGPIFQIQTLSDTIIVVSGHELVAEVCDETRFDKSIEGALAKVRAFAGDGLFTSETDEPNWKKAHNILMPTFSQRAMKDYHAMMVDIAVQLVQKWARLNPNENVDVPEDMTRLTLDTIGLCGFNYRFNSFYRETPHPFITSMTRALDEAMHQLQRLDIEDKLMWRTKRQFQHDIQSMFSLVDNIIAERKSSENQEENDLLSRMLNVQDPETGEKLDDENIRFQIITFLIAGHETTSGLLSFAIYFLLKNPDKLKKAYEEVDRVLTDSTPTYQQVMKLKYIRMILNESLRLWPTAPAFSLYAKEDTVIGGKYPIKKGEDRISVLIPQLHRDKDAWGDDVEEFQPERFEELDKVPHHAYKPFGNGQRACIGMQFALHEATLVMGMLLQHFEFIDYEDYQLDVKQTLTLKPGDFKIRIVPRNQTISHTTVLAPTEEKLKKHEIKKQVQKTPSIIGADNLSLLVLYGSDTGVAEGIARELADTASLEGVQTEVVALNDRIGSLPKEGAVLIVTSSYNGKPPSNAGQFVQWLEELKPDELKGVQYAVFGCGDHNWASTYQRIPRYIDEQMAQKGATRFSTRGEADASGDFEEQLEQWKQSMWSDAMKAFGLELNKNMEKERSTLSLQFVSRLGGSPLARTYEAVYASILENRELQSSSSERSTRHIEISLPEGATYKEGDHLGVLPINNEKNVNRILKRFGLNGKDQVILSASGRSVNHIPLDSPVRLYDLLSYSVEVQEAATRAQIREMVTFTACPPHKKELESLLEDGVYQEQILKKRISMLDLLEKYEACEIRFERFLELLPALKPRYYSISSSPLVAQDRLSITVGVVNAPAWSGEGTYEGVASNYLAQRHNKDEIICFIRTPQSNFQLPENPETPIIMVGPGTGIAPFRGFLQARRVQKQKGMKVGEAHLYFGCRHPEKDYLYRTELENDERDGLISLHTAFSRLEGHPKTYVQHVIKEDRIHLISLLDNGAHLYICGDGSKMAPDVEDTLCQAYQEIHEVSEQEARNWLDRLQEEGRYGKDVWAGI

SEQ ID NO: 31 - protein, 1064 aa, R. metallidurans CH34
MSTATPAAALEPIPRDPGWPIFGNLFQITPGEVGQHLLARSRHHDGIFELDFAGKRVPFVSSVALASELCDATRFRKIIGPPLSYLRDMAGDGLFTAHSDEPNWGCAHRILMPAFSQRAMKAYFDVMLRVANRLVDKWDRQGPDADIAVADDMTRLTLDTIALAGFGYDFASFASDELDPFVMAMVGALGEAMQKLTRLPIQDRFMGRAHRQAAEDIAYMRNLVDDVIRQRRVSPTSGMDLLNLMLEARDPETDRRLDDANIRNQVITFLIAGHETTSGLLTFALYELLRNPGVLAQAYAEVDTVLPGDALPVYADLARMPVLDRVLKETLRLWPTAPAFAVAPFDDVVLGGRYRLRKDRRISVVLTALHRDPKVWANPERFDIDRFLPENEAKLPAHAYMPFGQGERACIGRQFALTEAKLALALMLRNFAFQDPHDYQFRLKETLTIKPDQFVLRVRRRRPHERFVTRQASQAVADAAQTDVRGHGQAMTVLCASSLGTARELAEQIHAGAIAAGFDAKLADLDDAVGVLPTSGLVVVVAATYNGRAPDSARKFEAMLDADDASGYRANGMRLALLGCGNSQWATYQAFPRRVFDFFITAGAVPLLPRGEADGNGDFDQAAERWLAQLWQALQADGAGTGGLGVDVQVRSMAAIRAETLPAGTQAFTVLSNDELVGDPSGLWDFSIEAPRTSTRDIRLQLPPGITYRTGDHIAVWPQNDAQLVSELCERLDLDPDAQATISAPHGMGRGLPIDQALPVRQLLTHFIELQDVVSRQTLRALAQATRCPFTKQSIEQLASDDAEHGYATKVVARRLGILDVLVEHPAIALTLQELLACTVPMRPRLYSIASSPLVSPDVATLLVGTVCAPALSGRGQFRGVASTWLQHLPPGARVSASIRTPNPPFAPDPDPAAPMLLIGPGTGIAPFRGFLEERALRKMAGNAVTPAQLYFGCRHPQHDWLYREDIERWAGQGVVEVHPAYSVVPDAPRYVQDLLWQRREQVWAQVRDGATIYVCGDGRRMAPAVRQTLIEIGMAQGGMTDKAASDWFGGLVAQGRYRQDVFN

SEQ ID NO: 32 - protein, 1120 aa, A. fumigatus Af293
MSESKTVPIPGPRGVPLLGNIYDIEQEVPLRSINLMADQYGPIYRLTTFGWSRVFVSTHELVDEVCDEERFTKVVTAGLNQIRNGVHDGLFTANFPGEENWAIAHRVLVPAFGPLSIRGMFDEMYDIATQLVMKWARHGPTVPIMVTDDFTRLTLDTIALCAMGTRFNSFYHEEMHPFVEAMVGLLQGSGDRARRPALLNNLPTSENSKYWDDIAFLRNLAQELVEARRKNPEDKKDLLNALILGRDPKTGKGLTDESIIDNMITFLIAGHETTSGLLSFLFYYLLKTPNAYKKAQEEVDSVVGRRKITVEDMSRLPYLNAVMRETLRLRSTAPLIAVHAHPEKNKEDPVTLGGGKYVLNKDEPIVIILDKLHRDPQVYGPDAEEFKPERMLDENFEKLPKNAWKPFGNGMRACIGRPFAWQEALLVVAILLQNFNFQMDDPSYNLHIKQTLTIKPKDFHMRATLRHGLDATKLGIALSGSADRAPPESSGAASRVRKQATPPAGQLKPMHIFFGSNTGTCETFARRLADDAVGYGFAADVQSLDSAMQNVPKDEPVVFITASYEGQPPDNAAHFFEWLSALKENELEGVNYAVFGCGHHDWQATFHRIPKAVNQLVAEHGGNRLCDLGLADAANSDMFTDFDSWGESTFWPAITSKFGGGKSDEPKPSSSLQVEVSTGMRASTLGLQLQEGLVIDNQLLSAPDVPAKRMIRFKLPSDMSYRCGDYLAVLPVNPTSVVRRAIRRFDLPWDAMLTIRKPSQAPKGSTSIPLDTPISAFELLSTYVELSQPASKRDLTALADAAITDADAQAELRYLASSPTRFTEEIVKKRMSPLDLLIRYPSIKLPVGDFLAMLPPMRVRQYSISSSPLADPSECSITFSVLNAPALAAASLPPAERAEAEQYMGVASTYLSELKPGERAHIAVRPSHSGFKPPMDLKAPMIMACAGSGLAPFRGFIMDRAEKIRGRRSSVGADGQLPEVEQPAKAILYVGCRTKGKDDIHATELAEWAQLGAVDVRWAYSRPEDGSKGRHVQDLMLEDREELVSLFDQGARIYVCGSTGVGNGVRQACKDIYLERRRQLRQAARERGEEVPAEEDEDAAAEQFLDNLRTKERYATDVFT

SEQ ID NO: 33 - protein, 1083 aa, A. nidulans FGSC A4
MAEIPEPKGLPLIGNIGTIDQEFPLGSMVALAEEHGEIYRLRFPGRTVVVVSTHALVNETCDEKRFRKSVNSALAHVREGVHDGLFTAKMGEVNWEIAHRVLMPAFGPLSIRGMFDEMHDIASQLALKWARYGPDCPIMVTDDFTRLTLDTLALCSMGYRFNSYYSPVLHPFIEAMGDFLTEAGEKPRRPPLPAVFFRNRDQKFQDDIAVLRDTAQGVLQARKEGKSDRNDLLSAMLRGVDSQTGQKMTDESIMDNLITFLIAGHETTSGLLSFVFYQLLKHPETYRTAQQEVDNVVGQGVIEVSHLSKLPYINSVLRETLRLNATIPLFTVEAFEDTLLAGKYPVKAGETIVNLLAKSHLDPEVYGEDALEFKPERMSDELFNARLKQFPSAWKPFGNGMRACIGRPFAWQEALLVMAMLLQNFDFSLADPNYDLKFKQTLTIKPKDMFMKARLRHGLTPTTLERRLAGLAVESATQDKIVTNPADNSVTGTRLTILYGSNSGTCETLARRIAADAPSKGFHVMRFDGLDSGRSALPTDHPVVIVTSSYEGQPPENAKQFVSWLEELEQQNESLQLKGVDFAVFGCFKEWAQTFHRIPKLVDSLLEKLGGSRLTDLGLADVSTDELFSTFETWADDVLWPRLVAQYGADGKTQAHGSSAGHEAASNAAVEVTVSNSRTQALRQDVGQAMVVETRLLTAESEKERRKKHLEIRLPDGVSYTAGDYLAVLPINPPETVRRAMRQFKLSWDAQITIAPSGPTTALPTDGPIAANDIFSTYVELSQPATRKDLRIMADATTDPDVQKILRTYANETYTAEILTKSISVLDILEQHPAIDLPLGTFLLMLPSMRMRQYSISSSPLLTPTTATITISVLDAPSRSRSNGSRHLGVATSYLDSLSVGDHLQVTVRKNPSSGFRLPSEPETTPMICIAAGSGIAPFRAFLQERAVMMEQDKDRKLAPALLFFGCRAPGIDDLYREQLEEWQARGVVDARWAFSRQSDDTKGCRHVDDRILADREDVVKLWRDGARVYVCGSGALAQSVRSAMVTVLRDEMETTGDGSDNGKAEKWFDEQRNVRYVMDVFD

SEQ ID NO: 34 - protein, 1054 aa, A. oryzae
MRQNDNEKQICPIPGPQGLPFLGNILDIDLDNGTMSTLKIAKTYYPIFKFTFAGETSIVINSVALLSELCDETRFHKHVSFGLELLRSGTHDGLFTAYDHEKNWELAHRLLVPAFGPLRIREMFPQMHDIAQQLCLKWQRYGPRRPLNLVDDFTRTTLDTIALCAMGYRFNSFYSEGDFHPFIKSMVRFLKEAETQATLPSFISNLRVRAKRRTQLDIDLMRTVCREIVTERRQTNLDHKNDLLDTMLTSRDSLSGDALSDESIIDNILTFLVAGHETTSGLLSFAVYYLLTTPDAMAKAAHEVDDVVGDQELTIEHLSMLKYLNAILRETLRLMPTAPGFSVTPYKPEIIGGKYEVKPGDSLDVFLAAVHRDPAVYGSDADEFRPERMSDEHFQKLPANSWKPFGNGKRSCIGRAFAWQEALMILALILQSFSLNLVDRGYTLKLKESLTIKPDNLWAYATPRPGRNVLHTRLALQTNSTHPEGLMSLKHETVESQPATILYGSNSGTCEALAHRLAIEMSSKGRFVCKVQPMDAIEHRRLPRGQPVIIITGSYDGRPPENARHFVKWLQSLKGNDLEGIQYAVFGCGLPGHHDWSTTFYKIPTLIDTIMAEHGGARLAPRGSADTAEDDPFAELESWSERSVWPGLEAAFDLVRHNSSDGTGKSTRITIRSPYTLRAAHETAVVHQVRVLTSAETTKKVHVELALPDTINYRPGDHLAILPLNSRQSVQRVLSLFQIGSDTILYMTSSSATSLPTDTPISAHDLLSGYVELNQVATPTSLRSLAAKATDEKTAEYLEALATDRYTTEVRGNHLSLLDILESYSVPSIEIQHYIQMLPLLRPRQYTISSSPRLNRGQASLTVSVMERADVGGPRNCAGVASNYLASCTPGSILRVSLRQANPDFRLPDESCSHPIIMVAAGSGIAPFRAFVQERSVRQKEGIILPPAFLFFGCRRADLDDLYREELDAFEEQGVVTLFRAFSRAQSESHGCKYVQDLLWMERVRVKTLWGQDAKVFVCGSVRMNEGVKAIISKIVSPTPTEELARRYIAETFI

SEQ ID NO: 35 - protein, 1103 aa, A. oryzae
MSTPKAEPVPIPGPRGVPLMGNILDIESEIPLRSLEMMADTYGPIYRLTTFGFSRCMISSHELAAEVFDEERFTKKIMAGLSELRHGIHDGLFTAHMGEENWEIAHRVLMPAFGPLNIQNMFDEMHDIATQLVMKWARQGPKQKIMVTDDFTRLTLDTIALCAMGTRFNSFYSEEMHPFVDAMVGMLKTAGDRSRRPGLVNNLPTTENNKYWEDIDYLRNLCKELVDTRKKNPTDKKDLLNALINGRDPKTGKGMSYDSIIDNMITFLIAGHETTSGSLSFAFYNMLKNPQAYQKAQEEVDRVIGRRRITVEDLQKLPYITAVMRETLRLTPTAPAIAVGPHPTKNHEDPVTLGNGKYVLGKDEPCALLLGKIQRDPKVYGPDAEEFKPERMLDEHFNKLPKHAWKPFGNGMRACIGRPFAWQEALLVIAMLLQNFNFQMDDPSYNIQLKQTLTIKPNHFYMRAALREGLDAVHLGSALSASSSEHADHAAGHGKAGAAKKGADLKPMHVYYGSNTGTCEAFARRLADDATSYGYSAEVESLDSAKDSIPKNGPVVFITASYEGQPPDNAAHFFEWLSALKGDKPLDGVNYAVFGCGHHDWQTTFYRIPKEVNRLVGENGANRLCEIGLADTANADIVTDFDTWGETSFWPAVAAKFGSNTQGSQKSSTFRVEVSSGHRATTLGLQLQEGLVVENTLLTQAGVPAKRTIRFKLPTDTQYKCGDYLAILPVNPSTVVRKVMSRFDLPWDAVLRIEKASPSSSKHISIPMDTQVSAYDLFATYVELSQPASKRDLAVLADAAAVDPETQAELQAIASDPARFAEISQKRISVLDLLLQYPSINLAIGDFVAMLPPMRVRQYSISSSPLVDPTECSITFSVLKAPSLAALTKEDEYLGVASTYLSELRSGERVQLSVRPSHTGFKPPTELSTPMIMACAGSGLAPFRGFVMDRAEKIRGRRSSGSMPEQPAKAILYAGCRTQGKDDIHADELAEWEKIGAVEVRRAYSRPSDGSKGTHVQDLMMEDKKELIDLFESGARIYVCGTPGVGNAVRDSIKSMFLERREEIRRIAKEKGEPVSDDDEETAFEKFLDDMKTKERYTTDIFA

SEQ ID NO: 36 - protein, 1066 aa, F. oxysporum
MAESVPIPEPPGYPLIGNLGEFTSNPLSDLNRLADTYGPIFRLRLGAKAPIFVSSNSLINEVCDEKRFKKTLKSVLSQVREGVHDGLFTAFEDEPNWGKAHRILVPAFGPLSIRGMFPEMHDIATQLCMKFARHGPRTPIDTSDNFTRLALDTLALCAMDFRFYSYYKEELHPFIEAMGDFLTESGNRNRRPPFAPNFLYRAANEKFYGDIALMKSVADEVVAARKASPSDRKDLLAAMLNGVDPQTGEKLSDENITNQLITFLIAGHETTSGTLSFAMYQLLKNPEAYSKVQKEVDEVVGRGPVLVEHLTKLPYISAVLRETLRLNSPITAFGLEAIDDTFLGGKYLVKKGEIVTALLSRGHVDPVVYGNDADKFIPERMLDDEFARLNKEYPNCWKPFGNGKRACIGRPFAWQESLLAMVVLFQNFNFTMTDPNYALEIKQTLTIKPDHFYINATLRHGMTPTELEHVLAGNGATSSSTHNIKAAANLDAKAGSGKPMAIFYGSNSGTCEALANRLASDAPSHGFSATTVGPLDQAKQNLPEDRPVVIVTASYEGQPPSNAAHFIKWMEDLDGNDMEKVSYAVFACGHHDWVETFHRIPKLVDSTLEKRGGTRLVPMGSADAATSDMFSDFEAWEDIVLWPGLKEKYKISDEESGGQKGLLVEVSTPRKTSLRQDVEEALVVAEKTLTKSGPAKKHIEIQLPSAMTYKAGDYLAILPLNPKSTVARVFRRFSLAWDSFLKIQSEGPTTLPTNVAISAFDVFSAYVELSQPATKRNILALAEATEDKDTIQELERLAGDAYQAEISPKRVSVLDLLEKFPAVALPISSYLAMLPPMRVRQYSISSSPFADPSKLTLTYSLLDAPSLSGQGRHVGVATNFLSHLTAGDKLHVSVRASSEAFHLPSDAEKTPIICVAAGTGLAPLRGFIQERAAMLAAGRTLAPALLFFGCRNPEIDDLYAEEFERWEKMGAVDVRRAYSRATDKSEGCKYVQDRVYHDRADVFKVWDQGAKVFICGSREIGKAVEDVCVRLAIEKAQQNGRDVTEEMARAWFERSRNERFATDVFD

SEQ ID NO: 37 - protein, 1115 aa, G. moniliformis
MSATALFTRRSVSTSNPELRPIPGPKPLPLLGNLFDFDFDNLTKSLGELGKIHGPIYSITFGASTEIMVTSREIAQELCDETRFCKLPGGALDVMKAVVGDGLFTAETSNPKWAIAHRIITPLFGAMRIRGMFDDMKDICEQMCLRWARFGPDEPLNVCDNMTKLTLDTIALCTIDYRFNSFYRENGAAHPFAEAVVDVMTESFDQSNLPDFVNNYVRFRAMAKFKRQAAELRRQTEELIAARRQNPVDRDDLLNAMLSAKDPKTGEGLSPESIVDNLLTFLIAGHETTSSLLSFCFYYLLENPHVLRRVQQEVDTVVGSDTITVDHLSSMPYLEAVLRETLRLRDPGPGFYVKPLKDEVVAGKYAVNKDQPLFIVFDSVHRDQSTYGADADEFRPERMLKDGFDKLPPCAWKPFGNGVRACVGRPFAMQQAILAVAMVLHKFDLVKDESYTLKYHVTMTVRPVGFTMKVRLRQGQRATDLAMGLHRGHSQEASAAASPSRASLKRLSSDVNGDDTDHKSQIAVLYASNSGSCEALAYRLAAEATERGFGIRAVDVVNNAIDRIPVGSPVILITASYNGEPADDAQEFVPWLKSLESGRLNGVKFAVFGNGHRDWANTLFAVPRLIDSELARCGAERVSLMGVSDTCDSSDPFSDFERWIDEKLFPELETPHGPGGVKNGDRAVPRQELQVSLGQPPRITMRKGYVRAIVTEARSLSSPGVPEKRHLELLLPKDFNYKAGDHVYILPRNSPRDVVRALSYFGLGEDTLITIRNTARKLSLGLPLDTPITATDLLGAYVELGRTASLKNLWTLVDAAGHGSRAALLSLTEPERFRAEVQDRHVSILDLLERFPDIDLSLSCFLPMLAQIRPRAYSFSSAPDWKPGHATLTYTVVDFATPATQGINGSSKSKAVGDGTAVVQRQGLASSYLSSLGPGTSLYVSLHRASPYFCLQKSTSLPVIMVGAGTGLAPFRAFLQERRMAAEGAKQRFGPALLFFGCRGPRLDSLYSVELEAYETIGLVQVRRAYSRDPSAQDAQGCKYVTDRLGKCRDEVARLWMDGAQVLVCGGKKMANDVLEVLGPMLLEIDQKRGETTAKTVVEWRARLDKSRYVEEVYV

SEQ ID NO: 38 - protein, 1069 aa, G. zeae PH1
MAESVPIPEPPGYPLIGNLGEFKTNPLNDLNRLADTYGPIFRLHLGSKTPTFVSSNAFINEVCDEKRFKKTLKSVLSVVREGVHDGLFTAFEDEPNWGKAHRILIPAFGPLSIRNMFPEMHEIANQLCMKLARHGPHTPVDASDNFTRLALDTLALCAMDFRFNSYYKEELHPFIEAMGDFLLESGNRNRRPAFAPNFLYRAANDKFYADIALMKSVADEVVATRKQNPTDRKDLLAAMLEGVDPQTGEKLSDDNITNQLITFLIAGHETTSGTLSFAMYHLLKNPEAYNKLQKEIDEVIGRDPVTVEHLTKLPYLSAVLRETLRISSPITGFGVEAIEDTFLGGKYLIKKGETVLSVLSRGHVDPVVYGPDAEKFVPERMLDDEFARLNKEFPNCWKPFGNGKRACIGRPFAWQESLLAMALLFQNFNFTQTDPNYELQIKQNLTIKPDNFFFNCTLRHGMTPTDLEGQLAGKGATTSIASHIKAPAASKGAKASNGKPMAIYYGSNSGTCEALANRLASDAAGHGFSASVIGTLDQAKQNLPEDRPVVIVTASYEGQPPSNAAHFIKWMEDLAGNEMEKVSYAVFGCGHHDWVDTFLRIPKLVDTTLEQRGGTRLVPMGSADAATSDMFSDFEAWEDTVLWPSLKEKYNVTDDEASGQRGLLVEVTTPRKTTLRQDVEEALVVSEKTLTKTGPAKKHIEIQLPSGMTYKAGDYLAILPLNPRKTVSRVFRRFSLAWDSFLKIQSDGPTTLPINIAISAFDVFSAYVELSQPATKRNILALSEATEDKATIQELEKLAGDAYQEDVSAKKVSVLDLLEKYPAVALPISSYLAMLPPMRVRQYSISSSPFADPSKLTLTYSLLDAPSLSGQGRHVGVATNFLSQLIAGDKLHISVRASSAAFHLPSDPETTPIICVAAGTGLAPFRGFIQERAAMLAAGRKLAPALLFFGCRDPENDDLYAEELARWEQMGAVDVRRAYSRATDKSEGCKYVQDRIYHDRADVFKVWDQGAKVFICGSREIGKAVEDICVRLAMERSEATQEGKGATEEKAREWFERSRNERFATDVFD

SEQ ID NO: 39 - protein, 1066 aa, G. zeae PH1
MAIKDGGKKSGQIPGPKGLPVLGNLFDLDLSDSLTSLINIGQKYAPIFSLELGGHREVMICSRDLLDELCDETRFHKIVTGGVDKLRPLAGDGLFTAQHGNHDWGIAHRILMPLFGPLKIREMFDDMQDVSEQLCLKWARLGPSATIDVANDFTRLTLDTIALCTMGYRFNSFYSNDKMHPFVDSMVAALIDADKQSMFPDFIGACRVKALSAFRKHAAIMKGTCNELIQERRKNPIEGTDLLTAMMEGKDPKTGEGMSDDLIVQNLITFLIAGHETTSGLLSFAFYYLLENPHTLEKARAEVDEVVGDQALNVDHLTKMPYVNMILRETLRLMPTAPGFFVTPHKDEIIGGKYAVPANESLFCFLHLIHRDPKVWGADAEEFRPERMADEFFEALPKNAWKPFGNGMRGCIGREFAWQEAKLITVMILQNFELSKADPSYKLKIKQSLTIKPDGFNMHAKLRNDRKVSGLFKAPSLSSQQPSLSSRQSINAINAKDLKPISIFYGSNTGTCEALAQKLSADCVASGFMPSKPLPLDMATKNLSKDGPNILLAASYDGRPSDNAEEFTKWAESLKPGELEGVQFAVFGCGHKDWVSTYFKIPKILDKCLADAGAERLVEIGLTDASTGRLYSDFDDWENQKLFTELSKRQGVTPTDDSHLELNVTVIQPQNNDMGGNFKRAEVVENTLLTYPGVSRKHSLLLKLPKDMEYTPGDHVLVLPKNPPQLVEQAMSCFGVDSDTALTISSKRPTFLPTDTPILISSLLSSLVELSQTVSRTSLKRLADFADDDDTKACVERIAGDDYTVEVEEQRMSLLDILRKYPGINMPLSTFLSMLPQMRPRTYSFASAPEWKQGHGMLLFSVVEAEEGTVSRPGGLATNYMAQLRQGDSILVEPRPCRPELRTTMMLPEPKVPIIMIAVGAGLAPFLGYLQKRFLQAQSQRTALPPCTLLFGCRGAKMDDICRAQLDEYSRAGVVSVHRAYSRDPDSQCKYVQGLVTKHSETLAKQWAQGAIVMVCSGKKVSDGVMNVLSPILFAEEKRSGMTGADSVDVWRQNVPKERMILEVFG

SEQ ID NO: 40 - protein, 1130 aa, M. grisea
MFFLSSSLAYMAATQSRDWASFGVSLPSTALGRHLQAAMPFLSEENHKSQGTVLIPDAQGPIPFLGSVPLVDPELPSQSLQRLARQYGEIYRFVIPGRQSPILVSTHALVNELCDEKRFKKKVAAALLGLREAIHDGLFTAHNDEPNWGIAHRILMPAFGPMAIKGMFDEMHDVASQMILKWARHGSTTPIMVSDDFTRLTLDTIALCSMGYRFNSFYHDSMHEFIEAMTCWMKESGNKTRRLLPDVFYRTTDKKWHDDAEILRRTADEVLKARKENPSGRKDLLTAMIEGVDPKTGGKLSDSSIIDNLITFLIAGHETTSGMLSFAFYLLLKNPTAYRKAQQEIDDLCGREPITVEHLSKMPYITAVLRETLRLYSTIPAFVVEAIEDTVVGGKYAIPKNHPIFLMIAESHRDPKVYGDDAQEFEPERMLDGQFERRNREFPNSWKPFGNGMRGCIGRAFAWQEALLITAMLLQNFNFVMHDPAYQLSIKENLTLKPDNFYMRAILRHGMSPTELERSISGVAPTGNKTPPRNATRTSSPDPEDGGIPMSIYYGSNSGTCESLAHKLAVDASAQGFKAETVDVLDAANQKLPAGNRGPVVLITASYEGLPPDNAKHFVEWLENLKGGDELVDTSYAVFGCGHQDWTKTFHRIPKLVDEKLAEHGAVRLAPLGLSNAAHGDMFVDFETWEFETLWPALADRYKTGAGRQDAAATDLTAALSQLSVEVSHPRAADLRQDVGEAVVVAARDLTAPGAPPKRHMEIRLPKTGGRVHYSAGDYLAVLPVNPKSTVERAMRRFGLAWDAHVTIRSGGRTTLPTGAPVSAREVLSSYVELTQPATKRGIAVLAGAVTGGPAAEQEQAKAALLDLAGDSYALEVSAKRVGVLDLLERFPACAVPFGTFLALLPPMRVRQYSISSSPLWNDEHATLTYSVLSAPSLADPARTHVGVASSYLAGLGEGDHLHVALRPSHVAFRLPSPETPVVCVCAGSGMAPFRAFAQERAALVGAGRKVAPLLLFFGCREPGVDDLYREELEGWEAKGVLSVRRAYSRRTEQSEGCRYVQDRLLKNRAEVKSLWSQDAKVFVCGSREVAEGVKEAMFKVVAGKEGSSEEVQAWYEEVRNVRYASDIFD

SEQ ID NO: 41 - protein, 1108 aa, Neurospora crassa
MSSDETPQTIPIPGPPGLPLVGNSFDIDTEFPLGSMLNFADQYGEIFRLNFPGRNTVFVTSQALVHELCDEKRFQKTVNSALHEIRHGIHDGLFTARNDEPNWGIAHRILMPAFGPMAIQNMFPEMHEIASQLALKWARHGPNQSIKVTDDFTRLTLDTIALCSMDYRFNSYYHDDMHPFIDAMASFLVESGNRSRRPALPAFMYSKVDRKFYDDIRVLRETAEGVLKSRKEHPSERKDLLTAMLDGVDPKTGGKLSDDSIIDNLITFLIAGHETTSGLLSFAFVQLLKNPETYRKAQKEVDDVCGKGPIKLEHMNKLHYIAAVLRETLRLCPTIPVIGVESKEDTVIGGKYEVSKGQPFALLFAKSHVDPAVYGDTANDFDPERMLDENFERLNKEFPDCWKPFGNGMRACIGRPFAWQEALLVMAVCLQNFNFMPEDPNYTLQYKQTLTTKPKGFYMRAMLRDGMSALDLERRLKGELVAPKPTAQGPVSGQPKKSGEGKPISIYYGSNTGTCETFAQRLASDAEAHGFTATIIDSLDAANQNLPKDRPVVFITASYEGQPPDNAALFVGWLESLTGNELEGVQYAVFGCGHHDWAQTFHRIPKLVDNTVSERGGDRICSLGLADAGKGEMFTEFEQWEDEVFWPAMEEKYEVSRKEDDNEALLQSGLTVNFSKPRSSTLRQDVQEAVVVDAKTITAPGAPPKRHIEVQLSSDSGAYRSGDYLAVLPINPKETVNRVMRRFQLAWDTNITIEASRQTTILPTGVPMPVHDVLGAYVELSQPATKKNILALAEAADNAETKATLRQLAGPEYTEKITSRRVSILDLLEQFPSIPLPFSSFLSLLPPMRVRQYSISSSPLWNPSHVTLTYSLLESPSLSNPDKKHVGVATSYLASLEAGDKLNVSIRPSHKAFHLPVDADKTPLIMIAAGSGLAPFRGFVQERAAQIAAGRSLAPAMLFYGCRHPEQDDLYRDEFDKWESIGAVSVRRAFSRCPESQETKGCKYVGDRLWEDREEVTGLWDRGAKVYVCGSREVGESVKKVVVRIALERQKMIVEAREKGELDSLPEGIVEGLKLKGLTVEDVEVSEERALKWFEGIRNERYATDVFD

SEQ ID NO: 42 - protein, 561 aa, Oryza sativa
MAAAAAAAVPCVPFLCPPPPPLVSPRLRRGHVRLRLRPPRSSGGGGGGGAGGDEPPITTSWVSPDWLTALSRSVATRLGGGDDSGIPVASAKLDDVRDLLGGALFLPLFKWFREEGPVYRLAAGPRDLVVVSDPAVARHVLRGYGSRYEKGLVAEVSEFLFGSGFAIAEGALWTVRRRSVVPSLHKRFLSVMVDRVFCKCAERLVEKLETSALSGKPVNMEARFSQMTLDVIGLSLFNYNFDSLTSDSPVIDAVYTALKEAELRSTDLLPYWKIDLLCKIVPRQIKAEKAVNIIRNTVEDLITKCKKIVDAENEQIEGEEYVNEADPSILRFLLASREEVTSVQLRDDLLSMLVAGHETTGSVLTWTIYLLSKDPAALRRAQAEVDRVLQGRLPRYEDLKELKYLMRCINESMRLYPHPPVLIRRAIVDDVLPGNYKIKAGQDIMISVYNIHRSPEVWDRADDFIPERFDLEGPVPNETNTEYRFIPFSGGPRKCVGDQFALLEAIVALAVVLQKMDIELVPDQKINMTTGATIHTTNGLYMNVSLRKVDREPDFALSGSR

SEQ ID NO: 43 - protein, 467 aa, Artificial Sequence
  Synthetic Chimeric heme enzyme C2G9
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIAVQLIQKWARLNPNEAVDVPGDMTRLTLDTIGLCGFNYRFNSYYRETPHPFINSMVRALDEAMHQMQRLDVQDKLMVRTKRQFRYDIQTMFSLVDRMIAERKANPDENIKDLLSLMLYAKDPVTGETLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPISKGQPVTVLIPKLHRDQNAWGPDAEDFRPERFEDPSSIPHHAYKPFGNGQRACIGMQFALHEATLVLGMILKYFTLIDHENYELDIKQTLTLKPGDFHISVQSRHQEAIHADVQAAE

SEQ ID NO: 44 - protein, 467 aa, Artificial Sequence
  Chimeric heme enzyme X7
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 45 - protein, 466 aa, Artificial Sequence
  Chimeric heme enzyme X7-12
MTIKEMPQPKTFGELKNLPLLNTDKPVQALMKIADELGEIFKFEAPGRVTRYLSSQRLIKEACDEERFDKSIEGALEKVRAFSGDGLATSWTHEPNWRKAHNILMPTFSQRAMKDYHEKMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 46 - protein, 465 aa, Artificial Sequence
  Chimeric heme enzyme C2E6
MTIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPST

SEQ ID NO: 47 - protein, 467 aa, Artificial Sequence
  Chimeric heme enzyme X7-9
MKQASAIPQPKTYGPLKNLPHLEKEQLSQSLWRIADELGPIFRFDFPGVSSVFVSGHNLVAEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDSIIAERRANGDQDEKDLLARMLNVEDPETGEKLDDENIRFQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 48 - protein, 467 aa, Artificial Sequence
  Chimeric heme enzyme C2B12
MKQASAIPQPKTYGPLKNLPHLEKEQLSQSLWRIADELGPIFRFDFPGVSSVFVSGHNLVAEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFATYFLLKHPDKLKKAYEEVDRVLTDAAPTYKQVLELTYIRMILNESLRLWPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 49 - protein, 467 aa, Artificial Sequence
  Chimeric heme enzyme TSP234
MKETSPIPQPKTFGPLGNLPLIDKDKPTLSLIKLAEEQGPIFQIHTPAGTTIVVSGHELVKEVCDEERFDKSIEGALEKVRAFSGDGLATSWTHEPNWRKAHNILMPTFSQRAMKDYHEKMVDIATQLIQKWSRLNPNEEIDVADDMTRLTLDTIGLCGFNYRFNSFYRDQPHPFITSMVRALDEAMNKLQRANPDDPAYDENKRQFQEDIKVMNDLVDRMIAERKANPDENIKDLLSLMLYAKDPVTGETLDDENIRYQIITFLIAGHETTSGLLSFAIYCLLTHPEKLKKAQEEADRVLTDDTPEYKQIQQLKYIRMVLNETLRLYPTAPAFSLYAKEDTVLGGEYPISKGQPVTVLIPKLHRDQNAWGPDAEDFRPERFEDPSSIPHHAYKPFGNGQRACIGMQFALQEATMVLGLVLKHFELINHTGYELKIKEALTIKPDDFKITVKPRKTAAINVQRKEQA

SEQ ID NO: 50 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQALKFXRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSMVRALDEXMNKLQRANPDDPAYDENKRQFQEDIKVMNDLVDKIIADRKAXGEQSDDLLTXMLNGKDPETGEPLDDXNIRYQIITFLXAGHEXTSGLLSFALYFLVKNPHVLQKXAEEAARVLVDPVPSYKQVKQLKYVGMVLNEALRLWPTXPAFSLYAKEDTVLGGEYPLEKGDEXMVLIPQLHRDKTXWGDDVEEFRPERFENPSAIPQHAFKPFGNGQRASIGQQFALHEATLVLGMMLKHFDFEDHTNYELDIKETXXLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 51 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDKIIADRKARGEQSDDLLTQMLNGKDPETGEPLDDGNIRYQIITFLFAGHEATSGLLSFALYFLVKNPHVLQKVAEEAARVLVDPVPSYKQVKQLKYVGMVLNEALRLWPTVPAFSLYAKEDTVLGGEYPLEKGDEVMVLIPQLHRDKTVWGDDVEEFRPERFENPSAIPQHAFKPFGNGQRASIGQQFALHEATLVLGMMLKHFDFEDHTNYELDIKETVSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 52 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQAXKFXRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSMVRAXDEXMNKLQRANPDDPAYDENKRQFQEDIKVMNDLVDKIIADRKAXGEQSDDLLTXMLNGKDPETGEPLDDXNIRYQIITFLXAGHEXTSGLLSFALYFLVKNPHVLQKXAEEAARVLVDPVPSYKQVKQLKYVGMVLNEALRLWPTAPAFSLYAKEDTVLGGEYPLEKGDEXMVLIPQLHRDKTXWGDDVEEFRPERFENPSAIPQHAFKPFGNGQRASIGQQFALHEATLVLGMMLKHFDFEDHTNYELDIKETLTLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 53 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQAAKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAADEVMNKLQRANPDDPAYDENKRQFQEDIKVMNDLVDKIIADRKARGEQSDDLLTQMLNGKDPETGEPLDDGNIRYQIITFLFAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRASIGQQFALHEATLVLGMMLKHFDFEDHTNYELDIKETLTLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 54 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQALKFXRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSMVRALDEXMNKLQRANPDDPAYDENKRQFQEDIKVMNDLVDKIIADRKAXGEQSDDLLTXMLNGKDPETGEPLDDXNIRYQIITFLXAGHEXTSGLLSFALYFLVKNPHVLQKXAEEAARVLVDPVPSYKQVKQLKYVGMVLNEALRLWPTXPAFSLYAKEDTVLGGEYPLEKGDEXMVLIPQLHRDKTXWGDDVEEFRPERFENPSAIPQHAFKPFGNGQRAXIGQQFALHEATLVLGMMLKHFDFEDHTNYELDIKETXXLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 55 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQALKFXRDFAGDGLXTSWTHEKNWKKAHNILLPSFSQQAMKGYHAMMVDIAVQLVQKWERLNADEHIEVXEDMTRLTLDTIGLCGFNYRFNSFYRDQPHPFIXSMVRALDEXMNKLQRANPDDPAYDENKRQFQEDIKVMNDLVDKIIADRKAXGEQSDDLLTXMLNGKDPETGEPLDDXNIRYQIITFLXAGHEXTSGLLSFALYFLVKNPHVLQKXAEEAARVLVDPVPSYKQVKQLKYVGMVLNEALRLWPTAPAFSLYAKEDTVLGGEYPLEKGDEXMVLIPQLHRDKTXWGDDVEEFRPERFENPSAIPQHAFKPFGNGQRACIGQQFALHEATLVLGMMLKHFDFEDHTNYELDIKETLXLKPXGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 56 - protein, 1048 aa, Artificial Sequence
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHEXTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRAXIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 57 - protein, 1048 aa, Artificial Seqeunce
  Variant of Bacillus megaterium Cytochrome P450
TIKEMPQPKTFGELKNLPLLNTDKPVQALMKIADELGEIFKFEAPGRVTRYLSSQRLIKEACDESRFDKNLSQALKFARDFAGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQFQEDIKVMNDLVDKIIADRKARGEQSDDLLTQMLNGKDPETGEPLDDGNIRYQIITFLIAGHEATSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTVWGDDVEEFRPERFENPSAIPQHAFKPFGNGQRAXIGQQFALHEATLVLGMMLKHFDFEDHTNYELDIKETLSLKPKGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG